(19) United States
(12) Patent Application Publication
(10) Pub. No.: US 2025/0041179 A1
(43) Pub. Date: Feb. 6, 2025

(54) ANTIMICROBIAL COMBINATIONS

(71) Applicant: SYMRISE AG, Holzminden (DE)

(72) Inventors: Florian GENRICH, Dassel (DE); Sabine LANGE, Holzminden (DE); Steffen NORDZIEKE, Göttingen (DE); Sebastian BRUNCKE, Höxter (DE)

(73) Assignee: SYMRISE AG, Holzminden (DE)

(21) Appl. No.: 18/687,161

(22) PCT Filed: Aug. 27, 2021
(86) PCT No.: PCT/EP2021/073771

§ 371 Date: Feb. 27, 2024

(51) Int. Cl. A61K 8/37; A61Q 17/00; A61Q 19/00

(52) U.S. Cl. CPC A61K 8/375; A61Q 17/005; A61Q 19/00; A61K 2800/10; A61K 2800/524

(57) ABSTRACT

The present invention primarily relates to the use of use of one or more compound(s) selected from the group as defined herein in constituent (i) to improve the antimicrobial effect of one or more compound(s) selected from the group as defined herein in constituent (ii) and/or the use of one or more compound(s) selected from the group as defined herein in constituent (ii) to improve the antimicrobial effect of one or more compound(s) selected from the group as defined herein in constituent (i). Moreover, the present invention relates to the use of a mixture comprising or consisting of constituents (i) and (ii) as defined herein for providing an antimicrobial effect, preferably to a solid or liquid personal care product or to a semi-finished product. The present invention further relates to a process for improving the antimicrobial effect of one or more compound(s) as defined in step a) and/or of one or more compound(s) as defined in step b) and a process for antimicrobial stablilisation of a personal care product or a semi-finished product as defined herein. It also relates to a mixture comprising or consisting of constituents (i) and (ii) as defined herein, preferably wherein the total amounts of constituents (i) and (ii), respectively, contained in the mixture are such that constituent (i) synergistically improves the antimicrobial effect of constituent (ii) and/or that constituent (ii) synergistically improve(s) the antimicrobial effect of constituent (i).

### FIELD OF THE DISCLOSURE

The present invention primarily relates to the use of use of one or more compound(s) selected from the group as defined herein in constituent (i) to improve the antimicrobial effect of one or more compound(s) selected from the group as defined herein in constituent (ii) and/or the use of one or more compound(s) selected from the group as defined herein in constituent (ii) to improve the antimicrobial effect of one or more compound(s) selected from the group as defined herein in constituent (i). Moreover, the present invention relates to the use of a mixture comprising or consisting of constituents (i) and (ii) as defined herein for providing an antimicrobial effect, preferably to a solid or liquid personal care product or to a semi-finished product. The present invention further relates to a process for improving the antimicrobial effect of one or more compound(s) as defined in step a) (cf. below) and/or of one or more compound(s) as defined in step b) (cf. below) and a process for antimicrobial stabilisation of a personal care product or a semi-finished product as defined herein. It also relates to a mixture comprising or consisting of constituents (i) and (ii) as defined herein, preferably wherein the total amounts of constituents (i) and (ii), respectively, contained in the mixture are such that constituent (i) synergistically improves the antimicrobial effect of constituent (ii) and/or that constituent (ii) synergistically improve(s) the antimicrobial effect of constituent (i).

Further aspects of the present invention will arise from the description below, in particular from the examples, as well as from the attached patent claims.

### BACKGROUND

Personal care products usually contain antimicrobial agents to increase their shelf-life and to guarantee safety for the consumer's health. The aim is to protect consumers of said products from potentially pathogenic microorganisms that might otherwise grow inside the products, and to stop or delay the biological and/or physicochemical deterioration of the product. The same rationale applies to semi-finished products that are used in the production of said personal care products.

The preservation of personal care products can be achieved by chemical, physical or physicochemical methods. The most common strategy is the incorporation of antimicrobial agents into said products. Common antimicrobial agents are, for example, parabens, formaldehyde releasers, isothiazolinones, organic acids, triclosan, phenoxyethanol and benzyl alcohol. Some antimicrobial agents, however, have the drawback that they might induce undesirable side effects in consumers. These effects might range from mild skin irritation to allergic reactions to the chemicals used.

Hence, it was an object of the present invention to provide new combinations of antimicrobial agents that are highly active against a range of common pathogens and, preferably, enable the reduction of the concentration of the antimicrobial agents in personal care products to a minimum while still ensuring the protection of said products against the growth of pathogenic microorganisms. It was a further object of the present invention to provide new combinations of antimicrobial agents that lead to a synergistic improvement of the antimicrobial activity of the resulting mixture.

Further objects underlying the present invention follow from the description and the patent claims below.

### DESCRIPTION

According to a first aspect of the present invention, the stated objects are achieved by the use of
(i) one or more compound(s) selected from the group consisting of 3-hydroxypropyl caprylate and 3-hydroxypropyl undecylenate
to improve the antimicrobial effect of
(ii) one or more compound(s) selected from the group consisting of piroctone olamine (CAS number 68890-66-4), climbazole (CAS number 38083-17-9), zinc pyrithione (CAS number 13463-41-7), salicylic acid and salts thereof, decylene glycol, o-cymen-5-ol (CAS number 3228-02-2 or 39660-61-2), selenium sulfide, coal tar, sulphur, ketoconazole (CAS number 65277-42-1), benzoic acid and salts thereof, preferably sodium benzoate, sorbic acid and salts thereof, preferably potassium sorbate, lactic acid and salts thereof, dehydroacetic acid and salts thereof, preferably sodium dehydroacetate, undecylenic acid and salts thereof, levulinic acid and salts thereof, preferably sodium levulinate, anisic acid and salts thereof, phytic acid and salts thereof, preferably sodium phytate, itaconic acid and salts thereof, MEA undecylenate (CAS number 56532-40-2), butyloctanoic acid and salts thereof, citric acid and salts thereof, hydroxyacetophenone (CAS number 99-93-4), ethylhexylglycerin, caprylhydroxamic acid and salts thereof, undecylenoyl glycine, capryloyl glycine, polyglyceryl-3 caprylate (CAS number 108777-93-1), glyceryl undecylenate, glyceryl caprylate, sorbitan caprylate, glyceryl laurate, phenethyl alcohol, tropolones (e.g. CAS number 533-75-5), 1,2-propanediol, 1,2-butanediol, 1,2-pentanediol (pentylene glycol), 1,2-hexanediol, 1,2-heptanediol, 1,2-octanediol, 1,2-nonanediol, 1,2-decanediol, 1,2-undecanediol, 1,2-dodecanediol, 1,2-tridecanediol, 2,3-pentanediol, 2,3-hexanediol, 2,3-heptanediol, 2,3-octanediol, 2,3-nonanediol, 2,3-decanediol, 2,3-undecanediol, 2,3-dodecanediol, 2,3-tridecanediol, 1,3-propanediol, 1,3-butanediol, 2-methyl-2,4-pentanediol, caprylyl glycol, farnesol, bisabolol, nerolidol, carvacrol, thymol, phenylpropanol, methylbenzyl alcohol, 2-benzylheptanol, 2-methyl 5-cyclohexylpentanol, dimethyl phenylbutanol, PCA ethyl cocoyl arginate (CAS number 95370-65-3), benzyl alcohol, phenoxyethanol, iodopropynyl butylcarbamate, hydroxyethoxyphenyl butanol, ethyl lauroyl arginate HCl, methylchloroisothiazolinone, methylisothiazolinone, methylparaben (CAS number 99-76-3) and salts thereof, preferably sodium methylparaben, ethylparaben (CAS number 120-47-8) and salts thereof, preferably sodium ethylparaben, propylparaben (CAS number 94-13-3) and salts thereof, preferably sodium propylparaben, isopropylparaben (CAS number 4191-73-5) and salts thereof, butylparaben (CAS number 94-26-8) and salts thereof, isobutylparaben (CAS number 4247-02-3) and salts thereof, DMDM hydantoin (CAS number 6440-58-0), diazolidinyl urea (CAS number 78491-02-8), imidazolidinyl urea (CAS number 39236-46-9), chlorphenesin (CAS number 104-29-0), disodium EDTA (CAS number 139-33-3 or 6381-92-6), tetrasodium glutamate diacetate (glutamic acid N,N-diacetic tetrasodium salt), trisodium ethylenediamine disuccinate, methylglycine N,N-diacetic trisodium salt, glucoheptonic sodium salt, ethanoldiglycinic disodium salt, sodium gluconate, tetrasodium iminodisuccinate, trisodium salt of methylglycine diacetic acid, polyaspartic acid sodium salt, phenoxyisopropanol, chlorhexidine (CAS number 55-56-1), chlorhexidine digluconate (CAS number 18472-51-0), triclocarban (CAS number 101-20-2 or 1322-40-3), triclosan (CAS number 3380-34-5), O-phenylphenol, 4-hydroxybenzoic acid, TEA-salicylate (CAS number 2174-16-5), benzyl salicylate, hexamidine (CAS number 3811-75-4), hexamidine diisethionate (CAS number 659-40-5), dichlorobenzyl alcohol, ethyl benzoate, sodium paraben (CAS number 114-63-6 or 85080-04-2), isothiazolinones, p-chloro-m-cresol, cetrimonium chloride (CAS number 112-02-7), cetrimonium bromide (CAS number 57-09-0), benzethonium chloride (CAS number 121-54-0), alkyltrimonium bromide (alkyltrimethylammonium bromide, Cetrimide mix, CAS number 8044-71-1), alkyltrimonium chloride, benzalkonium chloride (mixture of alkylbenzyldimethylammonium chlorides, CAS numbers 61789-71-7, 68391-01-5, 68424-85-1, 8001-54-5, 85409-22-9), benzalkonium bromide (CAS number 91080-29-4), myristamidopropyl PG-dimonium chloride phosphate (INCI Monograph ID 19552), guar hydroxypropyltrimonium chloride (CAS number 65497-29-2), hydroxypropyl guar hydroxypropyltrimonium chloride (CAS number 71329-50-5), polyquaternium-6 (CAS number 26062-79-3), polyquaternium-7 (CAS number 26590-05-6), polyquaternium-10 (CAS numbers 53568-66-4, 54351-50-7, 55353-19-0, 68610-92-4, 81859-24-7), polyquaternium-11 (CAS number 53633-54-8), polyquaternium-12 (CAS number 68877-50-9), polyquaternium-15 (CAS numbers 35429-19-7, 67504-24-9), methylheptylglycerin, sodium caproyl/lauroyl lactylate, polyglyceryl-10 laurate (CAS numbers 74504-64-6, 34406-66-1), glyceryl caprate (CAS numbers 11139-88-1, 26402-22-2), polyglyceryl-2 caprate (CAS number 156153-06-9), raspberry ketone (CAS number 5471-51-2), raspberry ketone methyl ether (CAS number 104-20-1), caprylyl caprylate (CAS number 2306-88-9 or INCI Monograph ID: 23727), coco-caprylate/caprate (CAS number 95912-86-0 (generic)), PEG-8 caprylate (INCI Monograph ID: 1970), dipropylene glycol caprylate (CAS number 220604-16-0), propylene glycol caprylate (CAS numbers 31565-12-5 and 68332-79-6), pyridoxine dicaprylate (CAS number 106483-04-9), glyceryl caprylate/caprate (INCI Monograph ID: 1080), methyl glucose caprylate/caprate (INCI Monograph ID: 15374), polyglyceryl-3 caprate/caprylate/succinate (CAS number 1798313-25-3 (generic)), polyglyceryl-2 caprylate (INCI Monograph ID: 4659), polyglyceryl-3 caprylate (CAS number 108777-93-1), polyglyceryl-4 caprylate (INCI Monograph ID: 24482), polyglyceryl-6 caprylate (INCI Monograph ID: 12561), polyglyceryl-10 caprylate (CAS number 51033-41-1), polyglyceryl-4 caprylate/caprate (INCI Monograph ID: 26393), polyglyceryl-6 caprylate/caprate (INCI Monograph ID: 26091), polyglyceryl-10 caprylate/caprate (INCI Monograph ID: 25512), propanediol dicaprylate/caprate (CAS number 1072005-10-7), xylityl caprate/caprylate (CAS number 2127407-61-6 (generic)), xylityl caprylate (INCI Monograph ID: 32988), xylityl sesquicaprylate (CAS number 181632-90-6), and hydroxyethoxyphenyl butanone (CAS number 569646-79-3),
and/or
the use of one or more compound(s) selected from the group as defined in constituent (ii) to improve the antimicrobial effect of one or more compound(s) selected from the group as defined in constituent (i).

In the context of the studies underlying the present invention and presented further below, it was surprisingly found that uses as defined herein lead to particularly advantageous antimicrobial properties of the obtained mixtures against various bacteria and fungi commonly found in personal care products.

Personal care products are preferably applied directly to, for example, the skin or hair of the consumer of the product. Hence, it is particularly advantageous to ensure that personal care products are protected against the growth of potentially pathogenic microorganisms. This ensures that the personal care product is safe to use for the consumer and can be stored for a sufficient amount of time without degrading.

In this context, it was also found that the compounds as defined in constituent (i) above, particularly 3-hydroxypropyl caprylate, is/are very well suitable for the antimicrobial stabilisation of (cosmetic) formulations, preferably of solid or liquid personal care products or semi-finished products as defined herein. Thus, said (cosmetic) formulations are protected from microbial spoilage and decay by 3-hydroxypropyl caprylate and/or 3-hydroxypropyl undecylenate. Preferably, said (cosmetic) formulations are protected from microbial spoilage and decay by 3-hydroxypropyl caprylate. Preferably, said (cosmetic) formulations are protected from microbial spoilage and decay by 3-hydroxypropyl undecylenate. Most preferably, said (cosmetic) formulations are protected from microbial spoilage and decay by a mixture of 3-hydroxypropyl caprylate and 3-hydroxypropyl undecylenate.

Within the framework of the present text, the term “to improve” means “to enhance”, preferably means “to synergistically enhance”.

The use of one or more compound(s) selected from the group consisting of 3-hydroxypropyl caprylate and 3-hydroxypropyl undecylenate (constituent (i)) to improve the antimicrobial effect of one or more compound(s) selected from the group as defined in constituent (ii) was found to be particularly advantageous, as it enables a minimisation of the concentration of the one or more compound(s) selected from the group as defined in constituent (ii) in personal care products while still achieving a sufficient antimicrobial effect.

According to a preferred embodiment of the use according to the invention, 3-hydroxypropyl caprylate is used to improve the antimicrobial effect of one or more compound(s) selected from the group as defined in constituent (ii).

According to a another preferred embodiment of the use according to the invention, 3-hydroxypropyl undecylenate is used to improve the antimicrobial effect of one or more compound(s) selected from the group as defined in constituent (ii).

According to a another preferred embodiment of the use according to the invention, both 3-hydroxypropyl caprylate and 3-hydroxypropyl undecylenate are used to improve the antimicrobial effect of one or more compound(s) selected from the group as defined in constituent (ii).

According to a another preferred embodiment of the use according to the invention, one or more compound(s) selected from the group consisting of 3-hydroxypropyl caprylate and 3-hydroxypropyl undecylenate (constituent (i)) are used to improve the antimicrobial effect of one, two, three, four, five, six, seven, eight, nine, ten or more compound(s) of constituent (ii) as defined herein.

According to another preferred embodiment of the use according to the invention, 3-hydroxypropyl caprylate is used to improve the antimicrobial effect of piroctone olamine or vice versa, preferably 3-hydroxypropyl caprylate is used to synergistically enhance the antimicrobial effect of piroctone olamine or vice versa.

According to another preferred embodiment of the use according to the invention, 3-hydroxypropyl caprylate is used to improve the antimicrobial effect of xylityl caprylate or vice versa, preferably 3-hydroxypropyl caprylate is used to synergistically enhance the antimicrobial effect of xylityl caprylate or vice versa.

According to another preferred embodiment of the use according to the invention, 3-hydroxypropyl caprylate is used to improve the antimicrobial effect of raspberry ketone or vice versa, preferably 3-hydroxypropyl caprylate is used to synergistically enhance the antimicrobial effect of raspberry ketone or vice versa.

According to another preferred embodiment of the use according to the invention, 3-hydroxypropyl caprylate is used to improve the antimicrobial effect of xylityl sesquicaprylate or vice versa, preferably 3-hydroxypropyl caprylate is used to synergistically enhance the antimicrobial effect of xylityl sesquicaprylate or vice versa.

According to another preferred embodiment of the use according to the invention, 3-hydroxypropyl caprylate is used to improve the antimicrobial effect of salicylic acid or vice versa, preferably 3-hydroxypropyl caprylate is used to synergistically enhance the antimicrobial effect of salicylic acid or vice versa.

According to another preferred embodiment of the use according to the invention, 3-hydroxypropyl caprylate is used to improve the antimicrobial effect of sodium benzoate or vice versa, preferably 3-hydroxypropyl caprylate is used to synergistically enhance the antimicrobial effect of sodium benzoate or vice versa.

According to another preferred embodiment of the use according to the invention, 3-hydroxypropyl caprylate is used to improve the antimicrobial effect of potassium sorbate or vice versa, preferably 3-hydroxypropyl caprylate is used to synergistically enhance the antimicrobial effect of potassium sorbate or vice versa.

According to another preferred embodiment of the use according to the invention, 3-hydroxypropyl caprylate is used to improve the antimicrobial effect of decylene glycol or vice versa, preferably 3-hydroxypropyl caprylate is used to synergistically enhance the antimicrobial effect of decylene glycol or vice versa.

According to another preferred embodiment of the use according to the invention, 3-hydroxypropyl caprylate is used to improve the antimicrobial effect of climbazole or vice versa, preferably 3-hydroxypropyl caprylate is used to synergistically enhance the antimicrobial effect of climbazole or vice versa. 3-Hydroxypropyl caprylate (CAS number 102731-54-4) refers to the monoester of the alcohol 1,3-propanediol with caprylic acid. 3-hydroxypropyl undecylenate refers to the monoester of the alcohol 1,3-propanediol with 10-undecylenic acid.

Myristamidopropyl PG-dimonium chloride phosphate is the quaternary ammonium salt that conforms generally to the following formula, where RCO— represents the myristoyl radical:

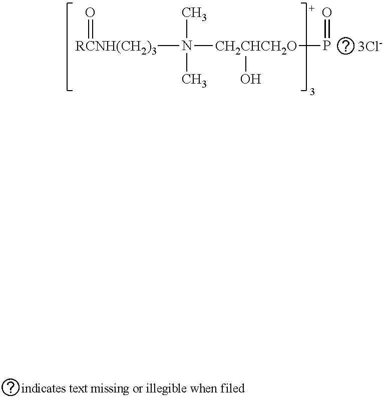

According to a preferred embodiment of the use according to the invention, the one or more compound(s) selected from the group as defined herein in constituent (i) synergistically improve(s) the antimicrobial effect of the one or more compound(s) selected from the group as defined herein in constituent (ii) and/or the one or more compound(s) selected from the group as defined herein in constituent (ii) synergistically improve(s) the antimicrobial effect of the one or more compound(s) selected from the group as defined herein in constituent (i).

Within the framework of the present text, the term “synergistically improve(s)” means that the antimicrobial effect brought about by the combination of constituent (i) and constituent (ii) is greater than the sum of the effect brought about by constituent (i) and of the effect brought about by constituent (ii).

According to a preferred embodiment of the use according to the invention, constituent (ii) is/are one or more compound(s) selected from the group consisting of piroctone olamine, climbazole, zinc pyrithione, salicylic acid and salts thereof, decylene glycol, o-cymen-5-ol, benzoic acid and salts thereof, preferably sodium benzoate, sorbic acid and salts thereof, preferably potassium sorbate, lactic acid and salts thereof, dehydroacetic acid and salts thereof, preferably sodium dehydroacetate, levulinic acid and salts thereof, preferably sodium levulinate, anisic acid and salts thereof, phytic acid and salts thereof, preferably sodium phytate, citric acid and salts thereof, hydroxyacetophenone, ethylhexylglycerin, caprylhydroxamic acid and salts thereof, undecylenoyl glycine, capryloyl glycine, polyglyceryl-3 caprylate, glyceryl undecylenate, glyceryl caprylate, sorbitan caprylate, 1,2-pentanediol (pentylene glycol), 1,2-hexanediol, 1,2-heptanediol, 1,2-octanediol, caprylyl glycol, 1,2-nonanediol, carvacrol, thymol, phenylpropanol, 2-methyl 5-cyclohexylpentanol, benzyl alcohol, phenoxyethanol, ethyl lauroyl arginate HCl, disodium EDTA, tetrasodium glutamate diacetate, trisodium ethylenediamine disuccinate, cetrimonium chloride, cetrimonium bromide, benzalkonium chloride, guar hydroxypropyltrimonium chloride, hydroxypropyl guar hydroxypropyltrimonium chloride, polyquaternium-6, polyquaternium-7, polyquaternium-10, and polyquaternium-12.

According to another preferred embodiment of the use according to the invention, constituent (ii) is/are one or more compound(s) selected from the group consisting of piroctone olamine, climbazole, zinc pyrithione, salicylic acid and salts thereof, decylene glycol, o-cymen-5-ol, benzoic acid and salts thereof, preferably sodium benzoate, sorbic acid and salts thereof, preferably potassium sorbate, lactic acid and salts thereof, dehydroacetic acid and salts thereof, preferably sodium dehydroacetate, levulinic acid and salts thereof, preferably sodium levulinate, anisic acid and salts thereof, phytic acid and salts thereof, preferably sodium phytate, citric acid and salts thereof, hydroxyacetophenone, ethylhexylglycerin, caprylhydroxamic acid and salts thereof, undecylenoyl glycine, capryloyl glycine, polyglyceryl-3 caprylate, glyceryl undecylenate, glyceryl caprylate, sorbitan caprylate, 1,2-pentanediol (pentylene glycol), 1,2-hexanediol, 1,2-heptanediol, 1,2-octanediol, caprylyl glycol, 1,2-nonanediol, phenylpropanol, benzyl alcohol, phenoxyethanol, ethyl lauroyl arginate HCl, disodium EDTA, tetrasodium glutamate diacetate, and trisodium ethylenediamine disuccinate.

According to a most preferred embodiment of the use according to the invention, constituent (ii) is/are one or more compound(s) selected from the group consisting of piroctone olamine, climbazole, zinc pyrithione, salicylic acid and salts thereof, decylene glycol, o-cymen-5-ol, benzoic acid and salts thereof, preferably sodium benzoate, sorbic acid and salts thereof, preferably potassium sorbate, lactic acid and salts thereof, dehydroacetic acid and salts thereof, preferably sodium dehydroacetate, levulinic acid and salts thereof, preferably sodium levulinate, anisic acid and salts thereof, phytic acid and salts thereof, preferably sodium phytate, 1,2-heptanediol, 1,2-octanediol, and 1,2-nonanediol.

Preferably, the improved antimicrobial effect is one or more effect(s) selected from the group consisting of an antibacterial effect, an antiviral effect, an antifungal effect, preferably an anti-yeast and/or an anti-mould effect.

Preferably, the improved antimicrobial effect is an antibacterial effect and/or an antifungal effect.

Most preferably, the improved antimicrobial effect is an antibacterial effect.

Most preferably, the improved antimicrobial effect is an antifungal effect.

According to a preferred embodiment of the use according to the invention, the antimicrobial effect is achieved against one or more microorganisms selected from the group consisting of bacteria and fungi, preferably gram positive and gram negative bacteria, yeasts and filamentous fungi, preferably selected from the group consisting of the genera Staphylococcus, Escherichia, Pseudomonas, Aspergillus, Candida, Pluralibacter, Burkholderia, Bacillus, Enterococcus, and Salmonella, most preferably selected from the group consisting of the species Staphylococcus aureus, Escherichia coli, Pseudomonas aeruginosa, Aspergillus brasiliensis, Candida albicans, Pluralibacter gergoviae, Burkholderia cepacia, Bacillus cereus, Enterococcus faecalis, and Salmonella enteritidis.

A second aspect of the present invention relates to the use of a mixture comprising or consisting of
(i) one or more compound(s) selected from the group consisting of 3-hydroxypropyl caprylate and 3-hydroxypropyl undecylenate,
and
(ii) one or more compound(s) selected from the group consisting of piroctone olamine, climbazole, zinc pyrithione, salicylic acid and salts thereof, decylene glycol, o-cymen-5-ol, selenium sulfide, coal tar, sulphur, ketoconazole, benzoic acid and salts thereof, preferably sodium benzoate, sorbic acid and salts thereof, preferably potassium sorbate, lactic acid and salts thereof, dehydroacetic acid and salts thereof, preferably sodium dehydroacetate, undecylenic acid and salts thereof, levulinic acid and salts thereof, preferably sodium levulinate, anisic acid and salts thereof, phytic acid and salts thereof, preferably sodium phytate, itaconic acid and salts thereof, MEA undecylenate, butyloctanoic acid and salts thereof, citric acid and salts thereof, hydroxyacetophenone, ethylhexylglycerin, caprylhydroxamic acid and salts thereof, undecylenoyl glycine, capryloyl glycine, polyglyceryl-3 caprylate, glyceryl undecylenate, glyceryl caprylate, sorbitan caprylate, glyceryl laurate, phenethyl alcohol, tropolones, 1,2-propanediol, 1,2-butanediol, 1,2-pentanediol (pentylene glycol), 1,2-hexanediol, 1,2-heptanediol, 1,2-octanediol, 1,2-nonanediol, 1,2-decanediol, 1,2-undecanediol, 1,2-dodecanediol, 1,2-tridecanediol, 2,3-pentanediol, 2,3-hexanediol, 2,3-heptanediol, 2,3-octanediol, 2,3-nonanediol, 2,3-decanediol, 2,3-undecanediol, 2,3-dodecanediol, 2,3-tridecanediol, 1,3-propanediol, 1,3-butanediol, 2-methyl-2,4-pentanediol, caprylyl glycol, farnesol, bisabolol, nerolidol, carvacrol, thymol, phenylpropanol, methylbenzyl alcohol, 2-benzylheptanol, 2-methyl 5-cyclohexylpentanol, dimethyl phenylbutanol, PCA ethyl cocoyl arginate, benzyl alcohol, phenoxyethanol, iodopropynyl butylcarbamate, hydroxyethoxyphenyl butanol, ethyl lauroyl arginate HCl, methylchloroisothiazolinone, methylisothiazolinone, methylparaben and salts thereof, preferably sodium methylparaben, ethylparaben and salts thereof, preferably sodium ethylparaben, propylparaben and salts thereof, preferably sodium propylparaben, isopropylparaben and salts thereof, butylparaben and salts thereof, isobutylparaben and salts thereof, DMDM hydantoin, diazolidinyl urea, imidazolidinyl urea, chlorphenesin, disodium EDTA, tetrasodium glutamate diacetate, trisodium ethylenediamine disuccinate, methylglycine N,N-diacetic trisodium salt, glucoheptonic sodium salt, ethanoldiglycinic disodium salt, sodium gluconate, tetrasodium iminodisuccinate, trisodium salt of methylglycine diacetic acid, polyaspartic acid sodium salt, phenoxyisopropanol, chlorhexidine, chlorhexidine digluconate, triclocarban, triclosan, O-phenylphenol, 4-hydroxybenzoic acid, TEA-salicylate, benzyl salicylate, hexamidine, hexamidine diisethionate, dichlorobenzyl alcohol, ethyl benzoate, sodium paraben, isothiazolinones, p-chloro-m-cresol, cetrimonium chloride, cetrimonium bromide, benzethonium chloride, alkyltrimonium bromide, alkyltrimonium chloride, benzalkonium chloride, benzalkonium bromide, myristamidopropyl PG-dimonium chloride phosphate, guar hydroxypropyltrimonium chloride, hydroxypropyl guar hydroxypropyltrimonium chloride, polyquaternium-6, polyquaternium-7, polyquaternium-10, polyquaternium-11, polyquaternium-12, polyquaternium-15, methylheptylglycerin, sodium caproyl/lauroyl lactylate, polyglyceryl-10 laurate, glyceryl caprate, polyglyceryl-2 caprate, raspberry ketone, raspberry ketone methyl ether, caprylyl caprylate, coco-caprylate/caprate, PEG-8 caprylate, dipropylene glycol caprylate, propylene glycol caprylate, pyridoxine dicaprylate, glyceryl caprylate/caprate, methyl glucose caprylate/caprate, polyglyceryl-3 caprate/caprylate/succinate, polyglyceryl-2 caprylate, polyglyceryl-3 caprylate, polyglyceryl-4 caprylate, polyglyceryl-6 caprylate, polyglyceryl-10 caprylate, polyglyceryl-4 caprylate/caprate, polyglyceryl-6 caprylate/caprate, polyglyceryl-10 caprylate/caprate, propanediol dicaprylate/caprate, xylityl caprate/caprylate, xylityl caprylate, xylityl sesquicaprylate, and hydroxyethoxyphenyl butanone,
for providing an antimicrobial effect, preferably to a solid or liquid personal care product or to a semi-finished product.

A preferred embodiment relates to the use of a mixture comprising 3-hydroxypropyl caprylate and piroctone olamine for providing an antimicrobial effect, preferably to a solid or liquid personal care product or to a semi-finished product.

Another preferred embodiment relates to the use of a mixture comprising 3-hydroxypropyl caprylate and xylityl caprylate for providing an antimicrobial effect, preferably to a solid or liquid personal care product or to a semi-finished product.

Another preferred embodiment relates to the use of a mixture comprising 3-hydroxypropyl caprylate and raspberry ketone for providing an antimicrobial effect, preferably to a solid or liquid personal care product or to a semi-finished product.

Another preferred embodiment relates to the use of a mixture comprising 3-hydroxypropyl caprylate and xylityl sesquicaprylate for providing an antimicrobial effect, preferably to a solid or liquid personal care product or to a semi-finished product.

Another preferred embodiment relates to the use of a mixture comprising 3-hydroxypropyl caprylate and salicylic acid for providing an antimicrobial effect, preferably to a solid or liquid personal care product or to a semi-finished product.

Another preferred embodiment relates to the use of a mixture comprising 3-hydroxypropyl caprylate and sodium benzoate for providing an antimicrobial effect, preferably to a solid or liquid personal care product or to a semi-finished product.

Another preferred embodiment relates to the use of a mixture comprising 3-hydroxypropyl caprylate and potassium sorbate for providing an antimicrobial effect, preferably to a solid or liquid personal care product or to a semi-finished product.

Another preferred embodiment relates to the use of a mixture comprising 3-hydroxypropyl caprylate and decylene glycol for providing an antimicrobial effect, preferably to a solid or liquid personal care product or to a semi-finished product.

Another preferred embodiment relates to the use of a mixture comprising 3-hydroxypropyl caprylate and climbazole for providing an antimicrobial effect, preferably to a solid or liquid personal care product or to a semi-finished product.

Preferably, the total amounts of constituents (i) and (ii) contained in the used mixture as defined herein, respectively, are selected such that constituent (i) synergistically improves the antimicrobial effect of constituent (ii) and/or that constituent (ii) synergistically improve(s) the antimicrobial effect of constituent (i).

The use of a mixture according to the invention for providing an antimicrobial effect, preferably to a solid or liquid personal care product or to a semi-finished product, is particularly advantageous, since the concentration of the mixture in the personal care or semi-finished product can be kept to a minimum while still achieving a sufficient antimicrobial effect. More preferably, it enables a minimisation of the concentration of the one or more compound(s) selected from the group as defined in constituent (ii) in personal care or semi-finished products while still achieving a sufficient antimicrobial effect.

Preferably, the solid or liquid personal care product contains from 0.01 to 10 wt. %, preferably from 0.02 to 5 wt. %, more preferably from 0.05 to 3 wt. %, most preferably from 0.1 to 2 wt. %, of constituent (i), based on the total weight of the personal care product,
and/or
the solid or liquid personal care product contains from 0.001 to 20 wt. %, preferably from 0.005 to 15 wt. %, more preferably from 0.009 to 10 wt. %, most preferably from 0.01 to 5 wt. %, of constituent (ii), based on the total weight of the personal care product.

More preferably, the solid or liquid personal care product contains from 0.01 to 10 wt. %, preferably from 0.02 to 5 wt. %, more preferably from 0.05 to 3 wt. %, most preferably from 0.1 to 2 wt. %, of constituent (i), based on the total weight of the personal care product,
and
the solid or liquid personal care product contains from 0.001 to 20 wt. %, preferably from 0.005 to 15 wt. %, more preferably from 0.009 to 10 wt. %, most preferably from 0.01 to 5 wt. %, of constituent (ii), based on the total weight of the personal care product.

Preferably, the solid or liquid personal care product, preferably leave-on or rinse-off personal care product, is selected from the group consisting of shampoos, preferably anti-dandruff shampoos, cleansing products, preferably shower gels, body washes and soaps, wet wipes, emulsions, preferably oil-in-water or water-in-oil emulsions, surfactant-based systems, tonics, preferably aqueous or aqueous/ethanolic/glycolic solutions and gels, leave-on and rinse-off conditioners, hair butters and waxes, beard care and conditioners.

Within the framework of the present text, the term “aqueous/ethanolic/glycolic solutions” refers to solutions comprising water and ethanol, or to solutions comprising water and glycol, or to solutions comprising ethanol and glycol, or to solutions comprising water and ethanol and glycol.

Preferably, the personal care product is/are wet wipes, e.g. for baby care and/or skin care (also referred to as cleansing wipes, water wipes or skin care wipes). The non-woven qualities used for this application are made of polyester, viscose or polyester/viscose blends, cotton, cellulose (Lyocell) or blends of this materials. The liquids/juices, which are used to soak the respective non-woven qualities in to prepare the finished wipes, preferably are water-based formulations. They can either consist of pure water or water with the addition of antimicrobials, diols, solubilisers, fragrances, ethanol or mixtures thereof. Also oil-in-water/water-in-oil emulsions and mircoemulsions are suitable components for preparing the wet wipes. The non-woven qualities themselves represent materials with high surface areas. Thus, it is often challenging to ensure sufficient antimicrobial efficacy over the whole life time circle of the wet wipes. Suitable antimicrobials with good efficacy and liquid appearance like 3-hydroxypropyl caprylate and/or 3-hydroxypropyl undecylenate as defined herein in constituent (i) alone or in combination with one or more further antimicrobials as defined herein in constituent (ii) are highly appreciated and fulfill said requirements for all kinds of wipes. Due to a combination of compounds from constituents (i) and (ii), respectively, a synergistic effect with regard to antimicrobial efficacy can be achieved.

Thus, according to a preferred embodiment of the invention, one, two, three or more of the mixtures as defined herein below is/are used for providing an antimicrobial effect to wet wipes. Preferably, one, two, three or more of the mixtures as defined herein below is/are included into the liquids/juices, which are used to soak the respective non-woven qualities in to prepare the finished wipes.

An example for an aqueous/glycolic juice for wet wipes is shown in Table 1 below. A suitable amount of one, two, three or more of the mixtures as defined herein below can be further added to the juice as active ingredient(s)/antimicrobial(s) for providing an improved antimicrobial effect to the wet wipes.

TABLE 1
Aqueous/glycolic juice for wet wipes

| Ingredients | INCI | w/w % |
|---|---|---|
| Propylenglycol | Propylene Glycol | 8.0 |
| Solubiliser PN660352 | PEG-40 Hydrogenated | 2.0 |
| | Castor Oil, Trideceth-9 | |
| | Propylene Glycol, Water | |
| | (Aqua) | |
| Active | | — |
| ingredient/Antimicrobial | | |
| Water (ultrapure) | Water (Aqua) | Ad 100 |
| Citric Acid, 10% sol. | Citric Acid | 0.1 |
| Sum | | 100.0 |

A third aspect of the invention relates to a process for improving the antimicrobial effect of one or more compound(s) as defined in step a) (cf. below) and/or of one or more compound(s) as defined in step b) (cf. below) comprising or consisting of the following steps:
a) Providing one or more compound(s) selected from the group consisting of 3-hydroxypropyl caprylate and 3-hydroxypropyl undecylenate, and
b) providing one or more compound(s) selected from the group consisting of piroctone olamine, climbazole, zinc pyrithione, salicylic acid and salts thereof, decylene glycol, o-cymen-5-ol, selenium sulfide, coal tar, sulphur, ketoconazole, benzoic acid and salts thereof, preferably sodium benzoate, sorbic acid and salts thereof, preferably potassium sorbate, lactic acid and salts thereof, dehydroacetic acid and salts thereof, preferably sodium dehydroacetate, undecylenic acid and salts thereof, levulinic acid and salts thereof, preferably sodium levulinate, anisic acid and salts thereof, phytic acid and salts thereof, preferably sodium phytate, itaconic acid and salts thereof, MEA undecylenate, butyloctanoic acid and salts thereof, citric acid and salts thereof, hydroxyacetophenone, ethylhexylglycerin, caprylhydroxamic acid and salts thereof, undecylenoyl glycine, capryloyl glycine, polyglyceryl-3 caprylate, glyceryl undecylenate, glyceryl caprylate, sorbitan caprylate, glyceryl laurate, phenethyl alcohol, tropolones, 1,2-propanediol, 1,2-butanediol, 1,2-pentanediol (pentylene glycol), 1,2-hexanediol, 1,2-heptanediol, 1,2-octanediol, 1,2-nonanediol, 1,2-decanediol, 1,2-undecanediol, 1,2-dodecanediol, 1,2-tridecanediol, 2,3-pentanediol, 2,3-hexanediol, 2,3-heptanediol, 2,3-octanediol, 2,3-nonanediol, 2,3-decanediol, 2,3-undecanediol, 2,3-dodecanediol, 2,3-tridecanediol, 1,3-propanediol, 1,3-butanediol, 2-methyl-2,4-pentanediol, caprylyl glycol, farnesol, bisabolol, nerolidol, carvacrol, thymol, phenylpropanol, methylbenzyl alcohol, 2-benzylheptanol, 2-methyl 5-cyclohexylpentanol, dimethyl phenylbutanol, PCA ethyl cocoyl arginate, benzyl alcohol, phenoxyethanol, iodopropynyl butylcarbamate, hydroxyethoxyphenyl butanol, ethyl lauroyl arginate HCl, methylchloroisothiazolinone, methylisothiazolinone, methylparaben and salts thereof, preferably sodium methylparaben, ethylparaben and salts thereof, preferably sodium ethylparaben, propylparaben and salts thereof, preferably sodium propylparaben, isopropylparaben and salts thereof, butylparaben and salts thereof, isobutylparaben and salts thereof, DMDM hydantoin, diazolidinyl urea, imidazolidinyl urea, chlorphenesin, disodium EDTA, tetrasodium glutamate diacetate, trisodium ethylenediamine disuccinate, methylglycine N,N-diacetic trisodium salt, glucoheptonic sodium salt, ethanoldiglycinic disodium salt, sodium gluconate, tetrasodium iminodisuccinate, trisodium salt of methylglycine diacetic acid, polyaspartic acid sodium salt, phenoxyisopropanol, chlorhexidine, chlorhexidine digluconate, triclocarban, triclosan, O-phenylphenol, 4-hydroxybenzoic acid, TEA-salicylate, benzyl salicylate, hexamidine, hexamidine diisethionate, dichlorobenzyl alcohol, ethyl benzoate, sodium paraben, isothiazolinones, p-chloro-m-cresol, cetrimonium chloride, cetrimonium bromide, benzethonium chloride, alkyltrimonium bromide, alkyltrimonium chloride, benzalkonium chloride, benzalkonium bromide, myristamidopropyl PG-dimonium chloride phosphate, guar hydroxypropyltrimonium chloride, hydroxypropyl guar hydroxypropyltrimonium chloride, polyquaternium-6, polyquaternium-7, polyquaternium-10, polyquaternium-11, polyquaternium-12, polyquaternium-15, methylheptylglycerin, sodium caproyl/lauroyl lactylate, polyglyceryl-10 laurate, glyceryl caprate, polyglyceryl-2 caprate, raspberry ketone, raspberry ketone methyl ether, caprylyl caprylate, coco-caprylate/caprate, PEG-8 caprylate, dipropylene glycol caprylate, propylene glycol caprylate, pyridoxine dicaprylate, glyceryl caprylate/caprate, methyl glucose caprylate/caprate, polyglyceryl-3 caprate/caprylate/succinate, polyglyceryl-2 caprylate, polyglyceryl-3 caprylate, polyglyceryl-4 caprylate, polyglyceryl-6 caprylate, polyglyceryl-10 caprylate, polyglyceryl-4 caprylate/caprate, polyglyceryl-6 caprylate/caprate, polyglyceryl-10 caprylate/caprate, propanediol dicaprylate/caprate, xylityl caprate/caprylate, xylityl caprylate, xylityl sesquicaprylate, and hydroxyethoxyphenyl butanone,
c) mixing the compounds provided in steps a) and b).

According to a preferred embodiment of the process according to the invention, the one or more compound(s) provided in step b) is/are selected from the group consisting of piroctone olamine, climbazole, zinc pyrithione, salicylic acid and salts thereof, decylene glycol, o-cymen-5-ol, benzoic acid and salts thereof, preferably sodium benzoate, sorbic acid and salts thereof, preferably potassium sorbate, lactic acid and salts thereof, dehydroacetic acid and salts thereof, preferably sodium dehydroacetate, levulinic acid and salts thereof, preferably sodium levulinate, anisic acid and salts thereof, phytic acid and salts thereof, preferably sodium phytate, citric acid and salts thereof, hydroxyacetophenone, ethylhexylglycerin, caprylhydroxamic acid and salts thereof, undecylenoyl glycine, capryloyl glycine, polyglyceryl-3 caprylate, glyceryl undecylenate, glyceryl caprylate, sorbitan caprylate, 1,2-pentanediol (pentylene glycol), 1,2-hexanediol, 1,2-heptanediol, 1,2-octanediol, caprylyl glycol, 1,2-nonanediol, carvacrol, thymol, phenylpropanol, 2-methyl 5-cyclohexylpentanol, benzyl alcohol, phenoxyethanol, ethyl lauroyl arginate HCl, disodium EDTA, tetrasodium glutamate diacetate, trisodium ethylenediamine disuccinate, cetrimonium chloride, cetrimonium bromide (CAS number 57-09-0), benzalkonium chloride, guar hydroxypropyltrimonium chloride, hydroxypropyl guar hydroxypropyltrimonium chloride, polyquaternium-6, polyquaternium-7, polyquaternium-10, and polyquaternium-12.

According to another preferred embodiment of the process according to the invention, the one or more compound(s) provided in step b) is/are selected from the group consisting of piroctone olamine, climbazole, zinc pyrithione, salicylic acid and salts thereof, decylene glycol, o-cymen-5-ol, benzoic acid and salts thereof, preferably sodium benzoate, sorbic acid and salts thereof, preferably potassium sorbate, lactic acid and salts thereof, dehydroacetic acid and salts thereof, preferably sodium dehydroacetate, levulinic acid and salts thereof, preferably sodium levulinate, anisic acid and salts thereof, phytic acid and salts thereof, preferably sodium phytate, citric acid and salts thereof, hydroxyacetophenone, ethylhexylglycerin, caprylhydroxamic acid and salts thereof, undecylenoyl glycine, capryloyl glycine, polyglyceryl-3 caprylate, glyceryl undecylenate, glyceryl caprylate, sorbitan caprylate, 1,2-pentanediol (pentylene glycol), 1,2-hexanediol, 1,2-heptanediol, 1,2-octanediol, caprylyl glycol, 1,2-nonanediol, phenylpropanol, benzyl alcohol, phenoxyethanol, ethyl lauroyl arginate HCl, disodium EDTA, tetrasodium glutamate diacetate, and trisodium ethylenediamine disuccinate.

According to a most preferred embodiment of the process according to the invention, the one or more compound(s) provided in step b) is/are selected from the group consisting of piroctone olamine, climbazole, zinc pyrithione, salicylic acid and salts thereof, decylene glycol, o-cymen-5-ol, benzoic acid and salts thereof, preferably sodium benzoate, sorbic acid and salts thereof, preferably potassium sorbate, lactic acid and salts thereof, dehydroacetic acid and salts thereof, preferably sodium dehydroacetate, levulinic acid and salts thereof, preferably sodium levulinate, anisic acid and salts thereof, phytic acid and salts thereof, preferably sodium phytate, 1,2-heptanediol, 1,2-octanediol, and 1,2-nonanediol.

According to a preferred embodiment of the process according to the invention, 3-hydroxypropyl caprylate is provided in step a) and piroctone olamine is provided in step b).

According to a preferred embodiment of the process according to the invention, 3-hydroxypropyl caprylate is provided in step a) and xylityl caprylate is provided in step b).

According to a preferred embodiment of the process according to the invention, 3-hydroxypropyl caprylate is provided in step a) and raspberry ketone is provided in step b).

According to a preferred embodiment of the process according to the invention, 3-hydroxypropyl caprylate is provided in step a) and xylityl sesquicaprylate is provided in step b).

According to a preferred embodiment of the process according to the invention, 3-hydroxypropyl caprylate is provided in step a) and salicylic acid is provided in step b).

According to a preferred embodiment of the process according to the invention, 3-hydroxypropyl caprylate is provided in step a) and sodium benzoate is provided in step b).

According to a preferred embodiment of the process according to the invention, 3-hydroxypropyl caprylate is provided in step a) and potassium sorbate is provided in step b).

According to a preferred embodiment of the process according to the invention, 3-hydroxypropyl caprylate is provided in step a) and decylene glycol is provided in step b).

According to a preferred embodiment of the process according to the invention, 3-hydroxypropyl caprylate is provided in step a) and climbazole is provided in step b).

According to a preferred embodiment, the one or more compound(s) as provided in step a) of the process as defined herein synergistically improve(s) the antimicrobial effect of the one or more compound(s) as provided in step b) as defined herein and/or the one or more compound(s) as provided in step b) as defined herein synergistically improve(s) the antimicrobial effect of the one or more compound(s) as provided in step a) as defined herein.

Preferably, the antimicrobial effect brought about by the mixture obtained in step c) of the process as defined herein is greater than the sum of the effect brought about by the one or more compounds provided in step a) and the effect brought about by the one or more compound(s) provided in step b).

A fourth aspect of the present invention relates to a process for antimicrobial stablilisation of a solid or liquid personal care product (as defined herein), preferably a leave-on or rinse-off personal care product, or for antimicrobial stablilisation of a semi-finished product, comprising or consisting of the following steps:
a) Providing
   (i) one or more compound(s) selected from the group consisting of 3-hydroxypropyl caprylate and 3-hydroxypropyl undecylenate, and
   (ii) one or more compound(s) selected from the group consisting of piroctone olamine, climbazole, zinc pyrithione, salicylic acid and salts thereof, decylene glycol, o-cymen-5-ol, selenium sulfide, coal tar, sulphur, ketoconazole, benzoic acid and salts thereof, preferably sodium benzoate, sorbic acid and salts thereof, preferably potassium sorbate, lactic acid and salts thereof, dehydroacetic acid and salts thereof, preferably sodium dehydroacetate, undecylenic acid and salts thereof, levulinic acid and salts thereof, preferably sodium levulinate, anisic acid and salts thereof, phytic acid and salts thereof, preferably sodium phytate, itaconic acid and salts thereof, MEA undecylenate, butyloctanoic acid and salts thereof, citric acid and salts thereof, hydroxyacetophenone, ethylhexylglycerin, caprylhydroxamic acid and salts thereof, undecylenoyl glycine, capryloyl glycine, polyglyceryl-3 caprylate, glyceryl undecylenate, glyceryl caprylate, sorbitan caprylate, glyceryl laurate, phenethyl alcohol, tropolones, 1,2-propanediol, 1,2-butanediol, 1,2-pentanediol (pentylene glycol), 1,2-hexanediol, 1,2-heptanediol, 1,2-octanediol, 1,2-nonanediol, 1,2-decanediol, 1,2-undecanediol, 1,2-dodecanediol, 1,2-tridecanediol, 2,3-pentanediol, 2,3-hexanediol, 2,3-heptanediol, 2,3-octanediol, 2,3-nonanediol, 2,3-decanediol, 2,3-undecanediol, 2,3-dodecanediol, 2,3-tridecanediol, 1,3-propanediol, 1,3-butanediol, 2-methyl-2,4-pentanediol, caprylyl glycol, farnesol, bisabolol, nerolidol, carvacrol, thymol, phenylpropanol, methylbenzyl alcohol, 2-benzylheptanol, 2-methyl 5-cyclohexylpentanol, dimethyl phenylbutanol, PCA ethyl cocoyl arginate, benzyl alcohol, phenoxyethanol, iodopropynyl butylcarbamate, hydroxyethoxyphenyl butanol, ethyl lauroyl arginate HCl, methylchloroisothiazolinone, methylisothiazolinone, methylparaben and salts thereof, preferably sodium methylparaben, ethylparaben and salts thereof, preferably sodium ethylparaben, propylparaben and salts thereof, preferably sodium propylparaben, isopropylparaben and salts thereof, butylparaben and salts thereof, isobutylparaben and salts thereof, DMDM hydantoin, diazolidinyl urea, imidazolidinyl urea, chlorphenesin, disodium EDTA, tetrasodium glutamate diacetate, trisodium ethylenediamine disuccinate, methylglycine N,N-diacetic trisodium salt, glucoheptonic sodium salt, ethanoldiglycinic disodium salt, sodium gluconate, tetrasodium iminodisuccinate, trisodium salt of methylglycine diacetic acid, polyaspartic acid sodium salt, phenoxyisopropanol, chlorhexidine, chlorhexidine digluconate, triclocarban, triclosan, O-phenylphenol, 4-hydroxybenzoic acid, TEA-salicylate, benzyl salicylate, hexamidine, hexamidine diisethionate, dichlorobenzyl alcohol, ethyl benzoate, sodium paraben, isothiazolinones, p-chloro-m-cresol, cetrimonium chloride, cetrimonium bromide, benzethonium chloride, alkyltrimonium bromide, alkyltrimonium chloride, benzalkonium chloride, benzalkonium bromide, myristamidopropyl PG-dimonium chloride phosphate, guar hydroxypropyltrimonium chloride, hydroxypropyl guar hydroxypropyltrimonium chloride, polyquaternium-6, polyquaternium-7, polyquaternium-10, polyquaternium-11, polyquaternium-12, polyquaternium-15, methylheptylglycerin, sodium caproyl/lauroyl lactylate, polyglyceryl-10 laurate, glyceryl caprate, polyglyceryl-2 caprate, raspberry ketone, raspberry ketone methyl ether, caprylyl caprylate, coco-caprylate/caprate, PEG-8 caprylate, dipropylene glycol caprylate, propylene glycol caprylate, pyridoxine dicaprylate, glyceryl caprylate/caprate, methyl glucose caprylate/caprate, polyglyceryl-3 caprate/caprylate/succinate, polyglyceryl-2 caprylate, polyglyceryl-3 caprylate, polyglyceryl-4 caprylate, polyglyceryl-6 caprylate, polyglyceryl-10 caprylate, polyglyceryl-4 caprylate/caprate, polyglyceryl-6 caprylate/caprate, polyglyceryl-10 caprylate/caprate, propanediol dicaprylate/caprate, xylityl caprate/caprylate, xylityl caprylate, xylityl sesquicaprylate, and hydroxyethoxyphenyl butanone, or providing a mixture of constituents (i) and (ii), and
b) providing the remaining ingredients of the personal care or semi-finished product, and
c) mixing the compounds provided in steps a) and b).

According to a preferred embodiment of the process according to the invention, 3-hydroxypropyl caprylate and piroctone olamine are provided in step a).

According to a preferred embodiment of the process according to the invention, 3-hydroxypropyl caprylate and xylityl caprylate are provided in step a).

According to a preferred embodiment of the process according to the invention, 3-hydroxypropyl caprylate and raspberry ketone are provided in step a).

According to a preferred embodiment of the process according to the invention, 3-hydroxypropyl caprylate and xylityl sesquicaprylate are provided in step a).

According to a preferred embodiment of the process according to the invention, 3-hydroxypropyl caprylate and salicylic acid are provided in step a).

According to a preferred embodiment of the process according to the invention, 3-hydroxypropyl caprylate and sodium benzoate are provided in step a).

According to a preferred embodiment of the process according to the invention, 3-hydroxypropyl caprylate and potassium sorbate are provided in step a).

According to a preferred embodiment of the process according to the invention, 3-hydroxypropyl caprylate and decylene glycol are provided in step a).

According to a preferred embodiment of the process according to the invention, 3-hydroxypropyl caprylate and climbazole are provided in step a).

According to a preferred embodiment of the process according to the invention, the one or more compound(s) of constituent (i) as provided in step a) as defined herein synergistically improve(s) the antimicrobial effect of the one or more compound(s) of constituent (ii) as provided in step a) as defined herein and/or the one or more compound(s) of constituent (ii) as provided in step a) as defined herein synergistically improve(s) the antimicrobial effect of the one or more compound(s) of constituent (i) as provided in step a) as defined herein.

Preferably, the solid or liquid personal care product obtained in step c) of the process as defined herein contains from 0.01 to 10 wt. %, preferably from 0.02 to 5 wt. %, more preferably from 0.05 to 3 wt. %, most preferably from 0.1 to 2 wt. %, of constituent (i), based on the total weight of the personal care product,
and/or
the solid or liquid personal care product obtained in step c) of the process as defined herein contains from 0.001 to 20 wt. %, preferably from 0.005 to 15 wt. %, more preferably from 0.009 to 10 wt. %, most preferably from 0.01 to 5 wt. %, of constituent (ii), based on the total weight of the personal care product.

Most preferably, the solid or liquid personal care product obtained in step c) of the process as defined herein contains from 0.01 to 10 wt. %, preferably from 0.02 to 5 wt. %, more preferably from 0.05 to 3 wt. %, most preferably from 0.1 to 2 wt. %, of constituent (i), based on the total weight of the personal care product,
and
the solid or liquid personal care product obtained in step c) of the process as defined herein contains from 0.001 to 20 wt. %, preferably from 0.005 to 15 wt. %, more preferably from 0.009 to 10 wt. %, most preferably from 0.01 to 5 wt. %, of constituent (ii), based on the total weight of the personal care product.

A fifth aspect of the invention relates to a process for producing a solid or liquid personal care product (as defined herein), preferably a leave-on or rinse-off personal care product, or for producing a semi-finished product, comprising or consisting of the following steps:
a) Providing
   (i) one or more compound(s) selected from the group consisting of 3-hydroxypropyl caprylate and 3-hydroxypropyl undecylenate, and
   (ii) one or more compound(s) selected from the group consisting of piroctone olamine, climbazole, zinc pyrithione, salicylic acid and salts thereof, decylene glycol, o-cymen-5-ol, selenium sulfide, coal tar, sulphur, ketoconazole, benzoic acid and salts thereof, preferably sodium benzoate, sorbic acid and salts thereof, preferably potassium sorbate, lactic acid and salts thereof, dehydroacetic acid and salts thereof, preferably sodium dehydroacetate, undecylenic acid and salts thereof, levulinic acid and salts thereof, preferably sodium levulinate, anisic acid and salts thereof, phytic acid and salts thereof, preferably sodium phytate, itaconic acid and salts thereof, MEA undecylenate, butyloctanoic acid and salts thereof, citric acid and salts thereof, hydroxyacetophenone, ethylhexylglycerin, caprylhydroxamic acid and salts thereof, undecylenoyl glycine, capryloyl glycine, polyglyceryl-3 caprylate, glyceryl undecylenate, glyceryl caprylate, sorbitan caprylate, glyceryl laurate, phenethyl alcohol, tropolones, 1,2-propanediol, 1,2-butanediol, 1,2-pentanediol (pentylene glycol), 1,2-hexanediol, 1,2-heptanediol, 1,2-octanediol, 1,2-nonanediol, 1,2-decanediol, 1,2-undecanediol, 1,2-dodecanediol, 1,2-tridecanediol, 2,3-pentanediol, 2,3-hexanediol, 2,3-heptanediol, 2,3-octanediol, 2,3-nonanediol, 2,3-decanediol, 2,3-undecanediol, 2,3-dodecanediol, 2,3-tridecanediol, 1,3-propanediol, 1,3-butanediol, 2-methyl-2,4-pentanediol, caprylyl glycol, farnesol, bisabolol, nerolidol, carvacrol, thymol, phenylpropanol, methylbenzyl alcohol, 2-benzylheptanol, 2-methyl 5-cyclohexylpentanol, dimethyl phenylbutanol, PCA ethyl cocoyl arginate, benzyl alcohol, phenoxyethanol, iodopropynyl butylcarbamate, hydroxyethoxyphenyl butanol, ethyl lauroyl arginate HCl, methylchloroisothiazolinone, methylisothiazolinone, methylparaben and salts thereof, preferably sodium methylparaben, ethylparaben and salts thereof, preferably sodium ethylparaben, propylparaben and salts thereof, preferably sodium propylparaben, isopropylparaben and salts thereof, butylparaben and salts thereof, isobutylparaben and salts thereof, DMDM hydantoin, diazolidinyl urea, imidazolidinyl urea, chlorphenesin, disodium EDTA, tetrasodium glutamate diacetate, trisodium ethylenediamine disuccinate, methylglycine N,N-diacetic trisodium salt, glucoheptonic sodium salt, ethanoldiglycinic disodium salt, sodium gluconate, tetrasodium iminodisuccinate, trisodium salt of methylglycine diacetic acid, polyaspartic acid sodium salt, phenoxyisopropanol, chlorhexidine, chlorhexidine digluconate, triclocarban, triclosan, O-phenylphenol, 4-hydroxybenzoic acid, TEA-salicylate, benzyl salicylate, hexamidine, hexamidine diisethionate, dichlorobenzyl alcohol, ethyl benzoate, sodium paraben, isothiazolinones, p-chloro-m-cresol, cetrimonium chloride, cetrimonium bromide, benzethonium chloride, alkyltrimonium bromide, alkyltrimonium chloride, benzalkonium chloride, benzalkonium bromide, myristamidopropyl PG-dimonium chloride phosphate, guar hydroxypropyltrimonium chloride, hydroxypropyl guar hydroxypropyltrimonium chloride, polyquaternium-6, polyquaternium-7, polyquaternium-10, polyquaternium-11, polyquaternium-12, polyquaternium-15, methylheptylglycerin, sodium caproyl/lauroyl lactylate, polyglyceryl-10 laurate, glyceryl caprate, polyglyceryl-2 caprate, raspberry ketone, raspberry ketone methyl ether, caprylyl caprylate, coco-caprylate/caprate, PEG-8 caprylate, dipropylene glycol caprylate, propylene glycol caprylate, pyridoxine dicaprylate, glyceryl caprylate/caprate, methyl glucose caprylate/caprate, polyglyceryl-3 caprate/caprylate/succinate, polyglyceryl-2 caprylate, polyglyceryl-3 caprylate, polyglyceryl-4 caprylate, polyglyceryl-6 caprylate, polyglyceryl-10 caprylate, polyglyceryl-4 caprylate/caprate, polyglyceryl-6 caprylate/caprate, polyglyceryl-10 caprylate/caprate, propanediol dicaprylate/caprate, xylityl caprate/caprylate, xylityl caprylate, xylityl sesquicaprylate, and hydroxyethoxyphenyl butanone,
      or providing a mixture of constituents (i) and (ii), and
b) providing the remaining ingredients of the personal care or semi-finished product, and
c) mixing the compounds provided in steps a) and b).

A sixth aspect of the present invention relates to a mixture comprising or consisting of
(i) one or more compound(s) selected from the group consisting of 3-hydroxypropyl caprylate and 3-hydroxypropyl undecylenate, and
(ii) one or more compound(s) selected from the group consisting of piroctone olamine, climbazole, zinc pyrithione, salicylic acid and salts thereof, decylene glycol, o-cymen-5-ol, selenium sulfide, coal tar, sulphur, ketoconazole, benzoic acid and salts thereof, preferably sodium benzoate, sorbic acid and salts thereof, preferably potassium sorbate, lactic acid and salts thereof, dehydroacetic acid and salts thereof, preferably sodium dehydroacetate, undecylenic acid and salts thereof, levulinic acid and salts thereof, preferably sodium levulinate, anisic acid and salts thereof, phytic acid and salts thereof, preferably sodium phytate, itaconic acid and salts thereof, MEA undecylenate, butyloctanoic acid and salts thereof, citric acid and salts thereof, hydroxyacetophenone, ethylhexylglycerin, caprylhydroxamic acid and salts thereof, undecylenoyl glycine, capryloyl glycine, polyglyceryl-3 caprylate, glyceryl undecylenate, glyceryl caprylate, sorbitan caprylate, glyceryl laurate, phenethyl alcohol, tropolones, 1,2-propanediol, 1,2-butanediol, 1,2-pentanediol (pentylene glycol), 1,2-hexanediol, 1,2-heptanediol, 1,2-octanediol, 1,2-nonanediol, 1,2-decanediol, 1,2-undecanediol, 1,2-dodecanediol, 1,2-tridecanediol, 2,3-pentanediol, 2,3-hexanediol, 2,3-heptanediol, 2,3-octanediol, 2,3-nonanediol, 2,3-decanediol, 2,3-undecanediol, 2,3-dodecanediol, 2,3-tridecanediol, 1,3-propanediol, 1,3-butanediol, 2-methyl-2,4-pentanediol, caprylyl glycol, farnesol, bisabolol, nerolidol, carvacrol, thymol, phenylpropanol, methylbenzyl alcohol, 2-benzylheptanol, 2-methyl 5-cyclohexylpentanol, dimethyl phenylbutanol, PCA ethyl cocoyl arginate, benzyl alcohol, phenoxyethanol, iodopropynyl butylcarbamate, hydroxyethoxyphenyl butanol, ethyl lauroyl arginate HCl, methylchloroisothiazolinone, methylisothiazolinone, methylparaben and salts thereof, preferably sodium methylparaben, ethylparaben and salts thereof, preferably sodium ethylparaben, propylparaben and salts thereof, preferably sodium propylparaben, isopropylparaben and salts thereof, butylparaben and salts thereof, isobutylparaben and salts thereof, DMDM hydantoin, diazolidinyl urea, imidazolidinyl urea, chlorphenesin, disodium EDTA, tetrasodium glutamate diacetate, trisodium ethylenediamine disuccinate, methylglycine N,N-diacetic trisodium salt, glucoheptonic sodium salt, ethanoldiglycinic disodium salt, sodium gluconate, tetrasodium iminodisuccinate, trisodium salt of methylglycine diacetic acid, polyaspartic acid sodium salt, phenoxyisopropanol, chlorhexidine, chlorhexidine digluconate, triclocarban, triclosan, O-phenylphenol, 4-hydroxybenzoic acid, TEA-salicylate, benzyl salicylate, hexamidine, hexamidine diisethionate, dichlorobenzyl alcohol, ethyl benzoate, sodium paraben, isothiazolinones, p-chloro-m-cresol, cetrimonium chloride, cetrimonium bromide, benzethonium chloride, alkyltrimonium bromide, alkyltrimonium chloride, benzalkonium chloride, benzalkonium bromide, myristamidopropyl PG-dimonium chloride phosphate, guar hydroxypropyltrimonium chloride, hydroxypropyl guar hydroxypropyltrimonium chloride, polyquaternium-6, polyquaternium-7, polyquaternium-10, polyquaternium-11, polyquaternium-12, polyquaternium-15, methylheptylglycerin, sodium caproyl/lauroyl lactylate, polyglyceryl-10 laurate, glyceryl caprate, polyglyceryl-2 caprate, raspberry ketone, raspberry ketone methyl ether, caprylyl caprylate, coco-caprylate/caprate, PEG-8 caprylate, dipropylene glycol caprylate, propylene glycol caprylate, pyridoxine dicaprylate, glyceryl caprylate/caprate, methyl glucose caprylate/caprate, polyglyceryl-3 caprate/caprylate/succinate, polyglyceryl-2 caprylate, polyglyceryl-3 caprylate, polyglyceryl-4 caprylate, polyglyceryl-6 caprylate, polyglyceryl-10 caprylate, polyglyceryl-4 caprylate/caprate, polyglyceryl-6 caprylate/caprate, polyglyceryl-10 caprylate/caprate, propanediol dicaprylate/caprate, xylityl caprate/caprylate, xylityl caprylate, xylityl sesquicaprylate, and hydroxyethoxyphenyl butanone, preferably wherein the total amounts of constituents (i) and (ii), respectively, contained in the mixture are such that constituent (i) synergistically improves the antimicrobial effect of constituent (ii) and/or that constituent (ii) synergistically improve(s) the antimicrobial effect of constituent (i).

According to a preferred embodiment, the mixture comprises or consists of 3-hydroxypropyl caprylate and piroctone olamine, preferably wherein the total amounts of 3-hydroxypropyl caprylate and piroctone olamine, respectively, contained in the mixture are such that 3-hydroxypropyl caprylate synergistically improves the antimicrobial effect of piroctone olamine and/or that piroctone olamine synergistically improve(s) the antimicrobial effect of 3-hydroxypropyl caprylate.

According to a preferred embodiment, the mixture comprises or consists of 3-hydroxypropyl caprylate and climbazole, preferably wherein the total amounts of 3-hydroxypropyl caprylate and climbazole, respectively, contained in the mixture are such that 3-hydroxypropyl caprylate synergistically improves the antimicrobial effect of climbazole and/or that climbazole synergistically improve(s) the antimicrobial effect of 3-hydroxypropyl caprylate.

According to a preferred embodiment, the mixture comprises or consists of 3-hydroxypropyl caprylate and zinc pyrithione, preferably wherein the total amounts of 3-hydroxypropyl caprylate and zinc pyrithione, respectively, contained in the mixture are such that 3-hydroxypropyl caprylate synergistically improves the antimicrobial effect of zinc pyrithione and/or that zinc pyrithione synergistically improve(s) the antimicrobial effect of 3-hydroxypropyl caprylate.

According to a preferred embodiment, the mixture comprises or consists of 3-hydroxypropyl caprylate and salicylic acid (and/or salts thereof), preferably wherein the total amounts of 3-hydroxypropyl caprylate and salicylic acid (and/or salts thereof), respectively, contained in the mixture are such that 3-hydroxypropyl caprylate synergistically improves the antimicrobial effect of salicylic acid (and/or salts thereof) and/or that salicylic acid (and/or salts thereof) synergistically improve(s) the antimicrobial effect of 3-hydroxypropyl caprylate.

According to a preferred embodiment, the mixture comprises or consists of 3-hydroxypropyl caprylate and decylene glycol, preferably wherein the total amounts of 3-hydroxypropyl caprylate and decylene glycol, respectively, contained in the mixture are such that 3-hydroxypropyl caprylate synergistically improves the antimicrobial effect of decylene glycol and/or that decylene glycol synergistically improve(s) the antimicrobial effect of 3-hydroxypropyl caprylate.

According to a preferred embodiment, the mixture comprises or consists of 3-hydroxypropyl caprylate and o-cymen-5-ol, preferably wherein the total amounts of 3-hydroxypropyl caprylate and o-cymen-5-ol, respectively, contained in the mixture are such that 3-hydroxypropyl caprylate synergistically improves the antimicrobial effect of o-cymen-5-ol and/or that o-cymen-5-ol synergistically improve(s) the antimicrobial effect of 3-hydroxypropyl caprylate.

According to a preferred embodiment, the mixture comprises or consists of 3-hydroxypropyl caprylate and selenium sulfide, preferably wherein the total amounts of 3-hydroxypropyl caprylate and selenium sulfide, respectively, contained in the mixture are such that 3-hydroxypropyl caprylate synergistically improves the antimicrobial effect of selenium sulfide and/or that selenium sulfide synergistically improve(s) the antimicrobial effect of 3-hydroxypropyl caprylate.

According to a preferred embodiment, the mixture comprises or consists of 3-hydroxypropyl caprylate and coal tar, preferably wherein the total amounts of 3-hydroxypropyl caprylate and coal tar, respectively, contained in the mixture are such that 3-hydroxypropyl caprylate synergistically improves the antimicrobial effect of coal tar and/or that coal tar synergistically improve(s) the antimicrobial effect of 3-hydroxypropyl caprylate.

According to a preferred embodiment, the mixture comprises or consists of 3-hydroxypropyl caprylate and sulphur, preferably wherein the total amounts of 3-hydroxypropyl caprylate and sulphur, respectively, contained in the mixture are such that 3-hydroxypropyl caprylate synergistically improves the antimicrobial effect of sulphur and/or that sulphur synergistically improve(s) the antimicrobial effect of 3-hydroxypropyl caprylate.

According to a preferred embodiment, the mixture comprises or consists of 3-hydroxypropyl caprylate and ketoconazole, preferably wherein the total amounts of 3-hydroxypropyl caprylate and ketoconazole, respectively, contained in the mixture are such that 3-hydroxypropyl caprylate synergistically improves the antimicrobial effect of ketoconazole and/or that ketoconazole synergistically improve(s) the antimicrobial effect of 3-hydroxypropyl caprylate.

According to a preferred embodiment, the mixture comprises or consists of 3-hydroxypropyl caprylate and benzoic acid (and/or salts thereof, preferably sodium benzoate), preferably wherein the total amounts of 3-hydroxypropyl caprylate and benzoic acid (and/or salts thereof, preferably sodium benzoate), respectively, contained in the mixture are such that 3-hydroxypropyl caprylate synergistically improves the antimicrobial effect of benzoic acid (and/or salts thereof, preferably sodium benzoate) and/or that benzoic acid (and/or salts thereof, preferably sodium benzoate) synergistically improve(s) the antimicrobial effect of 3-hydroxypropyl caprylate.

According to a preferred embodiment, the mixture comprises or consists of 3-hydroxypropyl caprylate and sorbic acid (and/or salts thereof, preferably potassium sorbate), preferably wherein the total amounts of 3-hydroxypropyl caprylate and sorbic acid (and/or salts thereof, preferably potassium sorbate), respectively, contained in the mixture are such that 3-hydroxypropyl caprylate synergistically improves the antimicrobial effect of sorbic acid (and/or salts thereof, preferably potassium sorbate) and/or that sorbic acid (and/or salts thereof, preferably potassium sorbate) synergistically improve(s) the antimicrobial effect of 3-hydroxypropyl caprylate.

According to a preferred embodiment, the mixture comprises or consists of 3-hydroxypropyl caprylate and lactic acid (and/or salts thereof), preferably wherein the total amounts of 3-hydroxypropyl caprylate and lactic acid (and/or salts thereof), respectively, contained in the mixture are such that 3-hydroxypropyl caprylate synergistically improves the antimicrobial effect of lactic acid (and/or salts thereof) and/or that lactic acid (and/or salts thereof) synergistically improve(s) the antimicrobial effect of 3-hydroxypropyl caprylate.

According to a preferred embodiment, the mixture comprises or consists of 3-hydroxypropyl caprylate and dehydroacetic acid (and/or salts thereof, preferably sodium dehydroacetate), preferably wherein the total amounts of 3-hydroxypropyl caprylate and dehydroacetic acid (and/or salts thereof, preferably sodium dehydroacetate), respectively, contained in the mixture are such that 3-hydroxypropyl caprylate synergistically improves the antimicrobial effect of dehydroacetic acid (and/or salts thereof, preferably sodium dehydroacetate) and/or that dehydroacetic acid (and/or salts thereof, preferably sodium dehydroacetate) synergistically improve(s) the antimicrobial effect of 3-hydroxypropyl caprylate.

According to a preferred embodiment, the mixture comprises or consists of 3-hydroxypropyl caprylate and undecylenic acid (and/or salts thereof), preferably wherein the total amounts of 3-hydroxypropyl caprylate and undecylenic acid (and/or salts thereof), respectively, contained in the mixture are such that 3-hydroxypropyl caprylate synergistically improves the antimicrobial effect of undecylenic acid (and/or salts thereof) and/or that undecylenic acid (and/or salts thereof) synergistically improve(s) the antimicrobial effect of 3-hydroxypropyl caprylate.

According to a preferred embodiment, the mixture comprises or consists of 3-hydroxypropyl caprylate and levulinic acid (and/or salts thereof, preferably sodium levulinate), preferably wherein the total amounts of 3-hydroxypropyl caprylate and levulinic acid (and/or salts thereof, preferably sodium levulinate), respectively, contained in the mixture are such that 3-hydroxypropyl caprylate synergistically improves the antimicrobial effect of levulinic acid (and/or salts thereof, preferably sodium levulinate) and/or that levulinic acid (and/or salts thereof, preferably sodium levulinate) synergistically improve(s) the antimicrobial effect of 3-hydroxypropyl caprylate.

According to a preferred embodiment, the mixture comprises or consists of 3-hydroxypropyl caprylate and anisic acid (and/or salts thereof), preferably wherein the total amounts of 3-hydroxypropyl caprylate and anisic acid (and/or salts thereof), respectively, contained in the mixture are such that 3-hydroxypropyl caprylate synergistically improves the antimicrobial effect of anisic acid (and/or salts thereof) and/or that anisic acid (and/or salts thereof) synergistically improve(s) the antimicrobial effect of 3-hydroxypropyl caprylate.

According to a preferred embodiment, the mixture comprises or consists of 3-hydroxypropyl caprylate and phytic acid (and/or salts thereof, preferably sodium phytate), preferably wherein the total amounts of 3-hydroxypropyl caprylate and phytic acid (and/or salts thereof, preferably sodium phytate), respectively, contained in the mixture are such that 3-hydroxypropyl caprylate synergistically improves the antimicrobial effect of phytic acid (and/or salts thereof, preferably sodium phytate) and/or that phytic acid (and/or salts thereof, preferably sodium phytate) synergistically improve(s) the antimicrobial effect of 3-hydroxypropyl caprylate.

According to a preferred embodiment, the mixture comprises or consists of 3-hydroxypropyl caprylate and itaconic acid (and/or salts thereof), preferably wherein the total amounts of 3-hydroxypropyl caprylate and itaconic acid (and/or salts thereof), respectively, contained in the mixture are such that 3-hydroxypropyl caprylate synergistically improves the antimicrobial effect of itaconic acid (and/or salts thereof) and/or that itaconic acid (and/or salts thereof) synergistically improve(s) the antimicrobial effect of 3-hydroxypropyl caprylate.

According to a preferred embodiment, the mixture comprises or consists of 3-hydroxypropyl caprylate and MEA undecylenate, preferably wherein the total amounts of 3-hydroxypropyl caprylate and MEA undecylenate, respectively, contained in the mixture are such that 3-hydroxypropyl caprylate synergistically improves the antimicrobial effect of MEA undecylenate and/or that MEA undecylenate synergistically improve(s) the antimicrobial effect of 3-hydroxypropyl caprylate.

According to a preferred embodiment, the mixture comprises or consists of 3-hydroxypropyl caprylate and butyloctanoic acid (and/or salts thereof), preferably wherein the total amounts of 3-hydroxypropyl caprylate and butyloctanoic acid (and/or salts thereof), respectively, contained in the mixture are such that 3-hydroxypropyl caprylate synergistically improves the antimicrobial effect of butyloctanoic acid (and/or salts thereof) and/or that butyloctanoic acid (and/or salts thereof) synergistically improve(s) the antimicrobial effect of 3-hydroxypropyl caprylate.

According to a preferred embodiment, the mixture comprises or consists of 3-hydroxypropyl caprylate and citric acid (and/or salts thereof), preferably wherein the total amounts of 3-hydroxypropyl caprylate and citric acid (and/or salts thereof), respectively, contained in the mixture are such that 3-hydroxypropyl caprylate synergistically improves the antimicrobial effect of citric acid (and/or salts thereof) and/or that citric acid (and/or salts thereof) synergistically improve(s) the antimicrobial effect of 3-hydroxypropyl caprylate.

According to a preferred embodiment, the mixture comprises or consists of 3-hydroxypropyl caprylate and hydroxyacetophenone, preferably wherein the total amounts of 3-hydroxypropyl caprylate and hydroxyacetophenone, respectively, contained in the mixture are such that 3-hydroxypropyl caprylate synergistically improves the antimicrobial effect of hydroxyacetophenone and/or that hydroxyacetophenone synergistically improve(s) the antimicrobial effect of 3-hydroxypropyl caprylate.

According to a preferred embodiment, the mixture comprises or consists of 3-hydroxypropyl caprylate and ethylhexylglycerin, preferably wherein the total amounts of 3-hydroxypropyl caprylate and ethylhexylglycerin, respectively, contained in the mixture are such that 3-hydroxypropyl caprylate synergistically improves the antimicrobial effect of ethylhexylglycerin and/or that ethylhexylglycerin synergistically improve(s) the antimicrobial effect of 3-hydroxypropyl caprylate.

According to a preferred embodiment, the mixture comprises or consists of 3-hydroxypropyl caprylate and caprylhydroxamic acid (and/or salts thereof), preferably wherein the total amounts of 3-hydroxypropyl caprylate and caprylhydroxamic acid (and/or salts thereof), respectively, contained in the mixture are such that 3-hydroxypropyl caprylate synergistically improves the antimicrobial effect of caprylhydroxamic acid (and/or salts thereof) and/or that caprylhydroxamic acid (and/or salts thereof) synergistically improve(s) the antimicrobial effect of 3-hydroxypropyl caprylate.

According to a preferred embodiment, the mixture comprises or consists of 3-hydroxypropyl caprylate and undecylenoyl glycine, preferably wherein the total amounts of 3-hydroxypropyl caprylate and undecylenoyl glycine, respectively, contained in the mixture are such that 3-hydroxypropyl caprylate synergistically improves the antimicrobial effect of undecylenoyl glycine and/or that undecylenoyl glycine synergistically improve(s) the antimicrobial effect of 3-hydroxypropyl caprylate.

According to a preferred embodiment, the mixture comprises or consists of 3-hydroxypropyl caprylate and capryloyl glycine, preferably wherein the total amounts of 3-hydroxypropyl caprylate and capryloyl glycine, respectively, contained in the mixture are such that 3-hydroxypropyl caprylate synergistically improves the antimicrobial effect of capryloyl glycine and/or that capryloyl glycine synergistically improve(s) the antimicrobial effect of 3-hydroxypropyl caprylate.

According to a preferred embodiment, the mixture comprises or consists of 3-hydroxypropyl caprylate and polyglyceryl-3 caprylate, preferably wherein the total amounts of 3-hydroxypropyl caprylate and polyglyceryl-3 caprylate, respectively, contained in the mixture are such that 3-hydroxypropyl caprylate synergistically improves the antimicrobial effect of polyglyceryl-3 caprylate and/or that polyglyceryl-3 caprylate synergistically improve(s) the antimicrobial effect of 3-hydroxypropyl caprylate.

According to a preferred embodiment, the mixture comprises or consists of 3-hydroxypropyl caprylate and glyceryl undecylenate, preferably wherein the total amounts of 3-hydroxypropyl caprylate and glyceryl undecylenate, respectively, contained in the mixture are such that 3-hydroxypropyl caprylate synergistically improves the antimicrobial effect of glyceryl undecylenate and/or that glyceryl undecylenate synergistically improve(s) the antimicrobial effect of 3-hydroxypropyl caprylate.

According to a preferred embodiment, the mixture comprises or consists of 3-hydroxypropyl caprylate and glyceryl caprylate, preferably wherein the total amounts of 3-hydroxypropyl caprylate and glyceryl caprylate, respectively, contained in the mixture are such that 3-hydroxypropyl caprylate synergistically improves the antimicrobial effect of glyceryl caprylate and/or that glyceryl caprylate synergistically improve(s) the antimicrobial effect of 3-hydroxypropyl caprylate.

According to a preferred embodiment, the mixture comprises or consists of 3-hydroxypropyl caprylate and sorbitan caprylate, preferably wherein the total amounts of 3-hydroxypropyl caprylate and sorbitan caprylate, respectively, contained in the mixture are such that 3-hydroxypropyl caprylate synergistically improves the antimicrobial effect of sorbitan caprylate and/or that sorbitan caprylate synergistically improve(s) the antimicrobial effect of 3-hydroxypropyl caprylate.

According to a preferred embodiment, the mixture comprises or consists of 3-hydroxypropyl caprylate and glyceryl laurate, preferably wherein the total amounts of 3-hydroxypropyl caprylate and glyceryl laurate, respectively, contained in the mixture are such that 3-hydroxypropyl caprylate synergistically improves the antimicrobial effect of glyceryl laurate and/or that glyceryl laurate synergistically improve(s) the antimicrobial effect of 3-hydroxypropyl caprylate.

According to a preferred embodiment, the mixture comprises or consists of 3-hydroxypropyl caprylate and phenethyl alcohol, preferably wherein the total amounts of 3-hydroxypropyl caprylate and phenethyl alcohol, respectively, contained in the mixture are such that 3-hydroxypropyl caprylate synergistically improves the antimicrobial effect of phenethyl alcohol and/or that phenethyl alcohol synergistically improve(s) the antimicrobial effect of 3-hydroxypropyl caprylate.

According to a preferred embodiment, the mixture comprises or consists of 3-hydroxypropyl caprylate and tropolones, preferably wherein the total amounts of 3-hydroxypropyl caprylate and tropolones, respectively, contained in the mixture are such that 3-hydroxypropyl caprylate synergistically improves the antimicrobial effect of tropolones and/or that tropolones synergistically improve(s) the antimicrobial effect of 3-hydroxypropyl caprylate.

According to a preferred embodiment, the mixture comprises or consists of 3-hydroxypropyl caprylate and 1,2-propanediol, preferably wherein the total amounts of 3-hydroxypropyl caprylate and 1,2-propanediol, respectively, contained in the mixture are such that 3-hydroxypropyl caprylate synergistically improves the antimicrobial effect of 1,2-propanediol and/or that 1,2-propanediol synergistically improve(s) the antimicrobial effect of 3-hydroxypropyl caprylate.

According to a preferred embodiment, the mixture comprises or consists of 3-hydroxypropyl caprylate and 1,2-butanediol, preferably wherein the total amounts of 3-hydroxypropyl caprylate and 1,2-butanediol, respectively, contained in the mixture are such that 3-hydroxypropyl caprylate synergistically improves the antimicrobial effect of 1,2-butanediol and/or that 1,2-butanediol synergistically improve(s) the antimicrobial effect of 3-hydroxypropyl caprylate.

According to a preferred embodiment, the mixture comprises or consists of 3-hydroxypropyl caprylate and 1,2-pentanediol, preferably wherein the total amounts of 3-hydroxypropyl caprylate and 1,2-pentanediol, respectively, contained in the mixture are such that 3-hydroxypropyl caprylate synergistically improves the antimicrobial effect of 1,2-pentanediol and/or that 1,2-pentanediol synergistically improve(s) the antimicrobial effect of 3-hydroxypropyl caprylate.

According to a preferred embodiment, the mixture comprises or consists of 3-hydroxypropyl caprylate and 1,2-hexanediol, preferably wherein the total amounts of 3-hydroxypropyl caprylate and 1,2-hexanediol, respectively, contained in the mixture are such that 3-hydroxypropyl caprylate synergistically improves the antimicrobial effect of 1,2-hexanediol and/or that 1,2-hexanediol synergistically improve(s) the antimicrobial effect of 3-hydroxypropyl caprylate.

According to a preferred embodiment, the mixture comprises or consists of 3-hydroxypropyl caprylate and 1,2-heptanediol, preferably wherein the total amounts of 3-hydroxypropyl caprylate and 1,2-heptanediol, respectively, contained in the mixture are such that 3-hydroxypropyl caprylate synergistically improves the antimicrobial effect of 1,2-heptanediol and/or that 1,2-heptanediol synergistically improve(s) the antimicrobial effect of 3-hydroxypropyl caprylate.

According to a preferred embodiment, the mixture comprises or consists of 3-hydroxypropyl caprylate and 1,2-octanediol, preferably wherein the total amounts of 3-hydroxypropyl caprylate and 1,2-octanediol, respectively, contained in the mixture are such that 3-hydroxypropyl caprylate synergistically improves the antimicrobial effect of 1,2-octanediol and/or that 1,2-octanediol synergistically improve(s) the antimicrobial effect of 3-hydroxypropyl caprylate.

According to a preferred embodiment, the mixture comprises or consists of 3-hydroxypropyl caprylate and 1,2-nonanediol, preferably wherein the total amounts of 3-hydroxypropyl caprylate and 1,2-nonanediol, respectively, contained in the mixture are such that 3-hydroxypropyl caprylate synergistically improves the antimicrobial effect of 1,2-nonanediol and/or that 1,2-nonanediol synergistically improve(s) the antimicrobial effect of 3-hydroxypropyl caprylate.

According to a preferred embodiment, the mixture comprises or consists of 3-hydroxypropyl caprylate and 1,2-decanediol, preferably wherein the total amounts of 3-hydroxypropyl caprylate and 1,2-decanediol, respectively, contained in the mixture are such that 3-hydroxypropyl caprylate synergistically improves the antimicrobial effect of 1,2-decanediol and/or that 1,2-decanediol synergistically improve(s) the antimicrobial effect of 3-hydroxypropyl caprylate.

According to a preferred embodiment, the mixture comprises or consists of 3-hydroxypropyl caprylate and 1,2-undecanediol, preferably wherein the total amounts of 3-hydroxypropyl caprylate and 1,2-undecanediol, respectively, contained in the mixture are such that 3-hydroxypropyl caprylate synergistically improves the antimicrobial effect of 1,2-undecanediol and/or that 1,2-undecanediol synergistically improve(s) the antimicrobial effect of 3-hydroxypropyl caprylate.

According to a preferred embodiment, the mixture comprises or consists of 3-hydroxypropyl caprylate and 1,2-dodecanediol, preferably wherein the total amounts of 3-hydroxypropyl caprylate and 1,2-dodecanediol, respectively, contained in the mixture are such that 3-hydroxypropyl caprylate synergistically improves the antimicrobial effect of 1,2-dodecanediol and/or that 1,2-dodecanediol synergistically improve(s) the antimicrobial effect of 3-hydroxypropyl caprylate.

According to a preferred embodiment, the mixture comprises or consists of 3-hydroxypropyl caprylate and 1,2-tridecanediol, preferably wherein the total amounts of 3-hydroxypropyl caprylate and 1,2-tridecanediol, respectively, contained in the mixture are such that 3-hydroxypropyl caprylate synergistically improves the antimicrobial effect of 1,2-tridecanediol and/or that 1,2-tridecanediol synergistically improve(s) the antimicrobial effect of 3-hydroxypropyl caprylate.

According to a preferred embodiment, the mixture comprises or consists of 3-hydroxypropyl caprylate and 2,3-pentanediol, preferably wherein the total amounts of 3-hydroxypropyl caprylate and 2,3-pentanediol, respectively, contained in the mixture are such that 3-hydroxypropyl caprylate synergistically improves the antimicrobial effect of 2,3-pentanediol and/or that 2,3-pentanediol synergistically improve(s) the antimicrobial effect of 3-hydroxypropyl caprylate.

According to a preferred embodiment, the mixture comprises or consists of 3-hydroxypropyl caprylate and 2,3-hexanediol, preferably wherein the total amounts of 3-hydroxypropyl caprylate and 2,3-hexanediol, respectively, contained in the mixture are such that 3-hydroxypropyl caprylate synergistically improves the antimicrobial effect of 2,3-hexanediol and/or that 2,3-hexanediol synergistically improve(s) the antimicrobial effect of 3-hydroxypropyl caprylate.

According to a preferred embodiment, the mixture comprises or consists of 3-hydroxypropyl caprylate and 2,3-heptanediol, preferably wherein the total amounts of 3-hydroxypropyl caprylate and 2,3-heptanediol, respectively, contained in the mixture are such that 3-hydroxypropyl caprylate synergistically improves the antimicrobial effect of 2,3-heptanediol and/or that 2,3-heptanediol synergistically improve(s) the antimicrobial effect of 3-hydroxypropyl caprylate.

According to a preferred embodiment, the mixture comprises or consists of 3-hydroxypropyl caprylate and 2,3-octanediol, preferably wherein the total amounts of 3-hydroxypropyl caprylate and 2,3-octanediol, respectively, contained in the mixture are such that 3-hydroxypropyl caprylate synergistically improves the antimicrobial effect of 2,3-octanediol and/or that 2,3-octanediol synergistically improve(s) the antimicrobial effect of 3-hydroxypropyl caprylate.

According to a preferred embodiment, the mixture comprises or consists of 3-hydroxypropyl caprylate and 2,3-nonanediol, preferably wherein the total amounts of 3-hydroxypropyl caprylate and 2,3-nonanediol, respectively, contained in the mixture are such that 3-hydroxypropyl caprylate synergistically improves the antimicrobial effect of 2,3-nonanediol and/or that 2,3-nonanediol synergistically improve(s) the antimicrobial effect of 3-hydroxypropyl caprylate.

According to a preferred embodiment, the mixture comprises or consists of 3-hydroxypropyl caprylate and 2,3-decanediol, preferably wherein the total amounts of 3-hydroxypropyl caprylate and 2,3-decanediol, respectively, contained in the mixture are such that 3-hydroxypropyl caprylate synergistically improves the antimicrobial effect of 2,3-decanediol and/or that 2,3-decanediol synergistically improve(s) the antimicrobial effect of 3-hydroxypropyl caprylate.

According to a preferred embodiment, the mixture comprises or consists of 3-hydroxypropyl caprylate and 2,3-undecanediol, preferably wherein the total amounts of 3-hydroxypropyl caprylate and 2,3-undecanediol, respectively, contained in the mixture are such that 3-hydroxypropyl caprylate synergistically improves the antimicrobial effect of 2,3-undecanediol and/or that 2,3-undecanediol synergistically improve(s) the antimicrobial effect of 3-hydroxypropyl caprylate.

According to a preferred embodiment, the mixture comprises or consists of 3-hydroxypropyl caprylate and 2,3-dodecanediol, preferably wherein the total amounts of 3-hydroxypropyl caprylate and 2,3-dodecanediol, respectively, contained in the mixture are such that 3-hydroxypropyl caprylate synergistically improves the antimicrobial effect of 2,3-dodecanediol and/or that 2,3-dodecanediol synergistically improve(s) the antimicrobial effect of 3-hydroxypropyl caprylate.

According to a preferred embodiment, the mixture comprises or consists of 3-hydroxypropyl caprylate and 2,3-tridecanediol, preferably wherein the total amounts of 3-hydroxypropyl caprylate and 2,3-tridecanediol, respectively, contained in the mixture are such that 3-hydroxypropyl caprylate synergistically improves the antimicrobial effect of 2,3-tridecanediol and/or that 2,3-tridecanediol synergistically improve(s) the antimicrobial effect of 3-hydroxypropyl caprylate.

According to a preferred embodiment, the mixture comprises or consists of 3-hydroxypropyl caprylate and 1,3-propanediol, preferably wherein the total amounts of 3-hydroxypropyl caprylate and 1,3-propanediol, respectively, contained in the mixture are such that 3-hydroxypropyl caprylate synergistically improves the antimicrobial effect of 1,3-propanediol and/or that 1,3-propanediol synergistically improve(s) the antimicrobial effect of 3-hydroxypropyl caprylate.

According to a preferred embodiment, the mixture comprises or consists of 3-hydroxypropyl caprylate and 1,3-butanediol, preferably wherein the total amounts of 3-hydroxypropyl caprylate and 1,3-butanediol, respectively, contained in the mixture are such that 3-hydroxypropyl caprylate synergistically improves the antimicrobial effect of 1,3-butanediol and/or that 1,3-butanediol synergistically improve(s) the antimicrobial effect of 3-hydroxypropyl caprylate.

According to a preferred embodiment, the mixture comprises or consists of 3-hydroxypropyl caprylate and 2-methyl-2,4-pentanediol, preferably wherein the total amounts of 3-hydroxypropyl caprylate and 2-methyl-2,4-pentanediol, respectively, contained in the mixture are such that 3-hydroxypropyl caprylate synergistically improves the antimicrobial effect of 2-methyl-2,4-pentanediol and/or that 2-methyl-2,4-pentanediol synergistically improve(s) the antimicrobial effect of 3-hydroxypropyl caprylate.

According to a preferred embodiment, the mixture comprises or consists of 3-hydroxypropyl caprylate and caprylyl glycol, preferably wherein the total amounts of 3-hydroxypropyl caprylate and caprylyl glycol, respectively, contained in the mixture are such that 3-hydroxypropyl caprylate synergistically improves the antimicrobial effect of caprylyl glycol and/or that caprylyl glycol synergistically improve(s) the antimicrobial effect of 3-hydroxypropyl caprylate.

According to a preferred embodiment, the mixture comprises or consists of 3-hydroxypropyl caprylate and farnesol, preferably wherein the total amounts of 3-hydroxypropyl caprylate and farnesol, respectively, contained in the mixture are such that 3-hydroxypropyl caprylate synergistically improves the antimicrobial effect of farnesol and/or that farnesol synergistically improve(s) the antimicrobial effect of 3-hydroxypropyl caprylate.

According to a preferred embodiment, the mixture comprises or consists of 3-hydroxypropyl caprylate and bisabolol, preferably wherein the total amounts of 3-hydroxypropyl caprylate and bisabolol, respectively, contained in the mixture are such that 3-hydroxypropyl caprylate synergistically improves the antimicrobial effect of bisabolol and/or that bisabolol synergistically improve(s) the antimicrobial effect of 3-hydroxypropyl caprylate.

According to a preferred embodiment, the mixture comprises or consists of 3-hydroxypropyl caprylate and nerolidol, preferably wherein the total amounts of 3-hydroxypropyl caprylate and nerolidol, respectively, contained in the mixture are such that 3-hydroxypropyl caprylate synergistically improves the antimicrobial effect of nerolidol and/or that nerolidol synergistically improve(s) the antimicrobial effect of 3-hydroxypropyl caprylate.

According to a preferred embodiment, the mixture comprises or consists of 3-hydroxypropyl caprylate and carvacrol, preferably wherein the total amounts of 3-hydroxypropyl caprylate and carvacrol, respectively, contained in the mixture are such that 3-hydroxypropyl caprylate synergistically improves the antimicrobial effect of carvacrol and/or that carvacrol synergistically improve(s) the antimicrobial effect of 3-hydroxypropyl caprylate.

According to a preferred embodiment, the mixture comprises or consists of 3-hydroxypropyl caprylate and thymol, preferably wherein the total amounts of 3-hydroxypropyl caprylate and thymol, respectively, contained in the mixture are such that 3-hydroxypropyl caprylate synergistically improves the antimicrobial effect of thymol and/or that thymol synergistically improve(s) the antimicrobial effect of 3-hydroxypropyl caprylate.

According to a preferred embodiment, the mixture comprises or consists of 3-hydroxypropyl caprylate and phenylpropanol, preferably wherein the total amounts of 3-hydroxypropyl caprylate and phenylpropanol, respectively, contained in the mixture are such that 3-hydroxypropyl caprylate synergistically improves the antimicrobial effect of phenylpropanol and/or that phenylpropanol synergistically improve(s) the antimicrobial effect of 3-hydroxypropyl caprylate.

According to a preferred embodiment, the mixture comprises or consists of 3-hydroxypropyl caprylate and methylbenzyl alcohol, preferably wherein the total amounts of 3-hydroxypropyl caprylate and methylbenzyl alcohol, respectively, contained in the mixture are such that 3-hydroxypropyl caprylate synergistically improves the antimicrobial effect of methylbenzyl alcohol and/or that methylbenzyl alcohol synergistically improve(s) the antimicrobial effect of 3-hydroxypropyl caprylate.

According to a preferred embodiment, the mixture comprises or consists of 3-hydroxypropyl caprylate and 2-benzylheptanol, preferably wherein the total amounts of 3-hydroxypropyl caprylate and 2-benzylheptanol, respectively, contained in the mixture are such that 3-hydroxypropyl caprylate synergistically improves the antimicrobial effect of 2-benzylheptanol and/or that 2-benzylheptanol synergistically improve(s) the antimicrobial effect of 3-hydroxypropyl caprylate.

According to a preferred embodiment, the mixture comprises or consists of 3-hydroxypropyl caprylate and 2-methyl 5-cyclohexylpentanol, preferably wherein the total amounts of 3-hydroxypropyl caprylate and 2-methyl 5-cyclohexylpentanol, respectively, contained in the mixture are such that 3-hydroxypropyl caprylate synergistically improves the antimicrobial effect of 2-methyl 5-cyclohexylpentanol and/or that 2-methyl 5-cyclohexylpentanol synergistically improve(s) the antimicrobial effect of 3-hydroxypropyl caprylate.

According to a preferred embodiment, the mixture comprises or consists of 3-hydroxypropyl caprylate and dimethyl phenylbutanol, preferably wherein the total amounts of 3-hydroxypropyl caprylate and dimethyl phenylbutanol, respectively, contained in the mixture are such that 3-hydroxypropyl caprylate synergistically improves the antimicrobial effect of dimethyl phenylbutanol and/or that dimethyl phenylbutanol synergistically improve(s) the antimicrobial effect of 3-hydroxypropyl caprylate.

According to a preferred embodiment, the mixture comprises or consists of 3-hydroxypropyl caprylate and PCA ethyl cocoyl arginate, preferably wherein the total amounts of 3-hydroxypropyl caprylate and PCA ethyl cocoyl arginate, respectively, contained in the mixture are such that 3-hydroxypropyl caprylate synergistically improves the antimicrobial effect of PCA ethyl cocoyl arginate and/or that PCA ethyl cocoyl arginate synergistically improve(s) the antimicrobial effect of 3-hydroxypropyl caprylate.

According to a preferred embodiment, the mixture comprises or consists of 3-hydroxypropyl caprylate and benzyl alcohol, preferably wherein the total amounts of 3-hydroxypropyl caprylate and benzyl alcohol, respectively, contained in the mixture are such that 3-hydroxypropyl caprylate synergistically improves the antimicrobial effect of benzyl alcohol and/or that benzyl alcohol synergistically improve(s) the antimicrobial effect of 3-hydroxypropyl caprylate.

According to a preferred embodiment, the mixture comprises or consists of 3-hydroxypropyl caprylate and phenoxyethanol, preferably wherein the total amounts of 3-hydroxypropyl caprylate and phenoxyethanol, respectively, contained in the mixture are such that 3-hydroxypropyl caprylate synergistically improves the antimicrobial effect of phenoxyethanol and/or that phenoxyethanol synergistically improve(s) the antimicrobial effect of 3-hydroxypropyl caprylate.

According to a preferred embodiment, the mixture comprises or consists of 3-hydroxypropyl caprylate and iodopropynyl butylcarbamate, preferably wherein the total amounts of 3-hydroxypropyl caprylate and iodopropynyl butylcarbamate, respectively, contained in the mixture are such that 3-hydroxypropyl caprylate synergistically improves the antimicrobial effect of iodopropynyl butylcarbamate and/or that iodopropynyl butylcarbamate synergistically improve(s) the antimicrobial effect of 3-hydroxypropyl caprylate.

According to a preferred embodiment, the mixture comprises or consists of 3-hydroxypropyl caprylate and hydroxyethoxyphenyl butanol, preferably wherein the total amounts of 3-hydroxypropyl caprylate and hydroxyethoxyphenyl butanol, respectively, contained in the mixture are such that 3-hydroxypropyl caprylate synergistically improves the antimicrobial effect of hydroxyethoxyphenyl butanol and/or that hydroxyethoxyphenyl butanol synergistically improve(s) the antimicrobial effect of 3-hydroxypropyl caprylate.

According to a preferred embodiment, the mixture comprises or consists of 3-hydroxypropyl caprylate and ethyl lauroyl arginate HCl, preferably wherein the total amounts of 3-hydroxypropyl caprylate and ethyl lauroyl arginate HCl, respectively, contained in the mixture are such that 3-hydroxypropyl caprylate synergistically improves the antimicrobial effect of ethyl lauroyl arginate HCl and/or that ethyl lauroyl arginate HCl synergistically improve(s) the antimicrobial effect of 3-hydroxypropyl caprylate.

According to a preferred embodiment, the mixture comprises or consists of 3-hydroxypropyl caprylate and methylchloroisothiazolinone, preferably wherein the total amounts of 3-hydroxypropyl caprylate and methylchloroisothiazolinone, respectively, contained in the mixture are such that 3-hydroxypropyl caprylate synergistically improves the antimicrobial effect of methylchloroisothiazolinone and/or that methylchloroisothiazolinone synergistically improve(s) the antimicrobial effect of 3-hydroxypropyl caprylate.

According to a preferred embodiment, the mixture comprises or consists of 3-hydroxypropyl caprylate and methylisothiazolinone, preferably wherein the total amounts of 3-hydroxypropyl caprylate and methylisothiazolinone, respectively, contained in the mixture are such that 3-hydroxypropyl caprylate synergistically improves the antimicrobial effect of methylisothiazolinone and/or that methylisothiazolinone synergistically improve(s) the antimicrobial effect of 3-hydroxypropyl caprylate.

According to a preferred embodiment, the mixture comprises or consists of 3-hydroxypropyl caprylate and methylparaben (and/or salts thereof, preferably sodium methylparaben), preferably wherein the total amounts of 3-hydroxypropyl caprylate and methylparaben (and/or salts thereof, preferably sodium methylparaben), respectively, contained in the mixture are such that 3-hydroxypropyl caprylate synergistically improves the antimicrobial effect of methylparaben (and/or salts thereof, preferably sodium methylparaben) and/or that methylparaben (and/or salts thereof, preferably sodium methylparaben) synergistically improve(s) the antimicrobial effect of 3-hydroxypropyl caprylate.

According to a preferred embodiment, the mixture comprises or consists of 3-hydroxypropyl caprylate and ethylparaben (and/or and salts thereof, preferably sodium ethylparaben), preferably wherein the total amounts of 3-hydroxypropyl caprylate and ethylparaben (and/or and salts thereof, preferably sodium ethylparaben), respectively, contained in the mixture are such that 3-hydroxypropyl caprylate synergistically improves the antimicrobial effect of ethylparaben (and/or and salts thereof, preferably sodium ethylparaben) and/or that ethylparaben (and/or and salts thereof, preferably sodium ethylparaben) synergistically improve(s) the antimicrobial effect of 3-hydroxypropyl caprylate.

According to a preferred embodiment, the mixture comprises or consists of 3-hydroxypropyl caprylate and propylparaben (and/or salts thereof, preferably sodium propylparaben), preferably wherein the total amounts of 3-hydroxypropyl caprylate and propylparaben (and/or salts thereof, preferably sodium propylparaben), respectively, contained in the mixture are such that 3-hydroxypropyl caprylate synergistically improves the antimicrobial effect of propylparaben (and/or salts thereof, preferably sodium propylparaben) and/or that propylparaben (and/or salts thereof, preferably sodium propylparaben) synergistically improve(s) the antimicrobial effect of 3-hydroxypropyl caprylate.

According to a preferred embodiment, the mixture comprises or consists of 3-hydroxypropyl caprylate and isopropylparaben (and/or salts thereof), preferably wherein the total amounts of 3-hydroxypropyl caprylate and isopropylparaben (and/or salts thereof), respectively, contained in the mixture are such that 3-hydroxypropyl caprylate synergistically improves the antimicrobial effect of isopropylparaben (and/or salts thereof) and/or that isopropylparaben (and/or salts thereof) synergistically improve(s) the antimicrobial effect of 3-hydroxypropyl caprylate.

According to a preferred embodiment, the mixture comprises or consists of 3-hydroxypropyl caprylate and butylparaben (and/or salts thereof), preferably wherein the total amounts of 3-hydroxypropyl caprylate and butylparaben (and/or salts thereof), respectively, contained in the mixture are such that 3-hydroxypropyl caprylate synergistically improves the antimicrobial effect of butylparaben (and/or salts thereof) and/or that butylparaben (and/or salts thereof) synergistically improve(s) the antimicrobial effect of 3-hydroxypropyl caprylate.

According to a preferred embodiment, the mixture comprises or consists of 3-hydroxypropyl caprylate and isobutylparaben (and/or salts thereof), preferably wherein the total amounts of 3-hydroxypropyl caprylate and isobutylparaben (and/or salts thereof), respectively, contained in the mixture are such that 3-hydroxypropyl caprylate synergistically improves the antimicrobial effect of isobutylparaben (and/or salts thereof) and/or that isobutylparaben (and/or salts thereof) synergistically improve(s) the antimicrobial effect of 3-hydroxypropyl caprylate.

According to a preferred embodiment, the mixture comprises or consists of 3-hydroxypropyl caprylate and DMDM hydantoin, preferably wherein the total amounts of 3-hydroxypropyl caprylate and DMDM hydantoin, respectively, contained in the mixture are such that 3-hydroxypropyl caprylate synergistically improves the antimicrobial effect of DMDM hydantoin and/or that DMDM hydantoin synergistically improve(s) the antimicrobial effect of 3-hydroxypropyl caprylate.

According to a preferred embodiment, the mixture comprises or consists of 3-hydroxypropyl caprylate and diazolidinyl urea, preferably wherein the total amounts of 3-hydroxypropyl caprylate and diazolidinyl urea, respectively, contained in the mixture are such that 3-hydroxypropyl caprylate synergistically improves the antimicrobial effect of diazolidinyl urea and/or that diazolidinyl urea synergistically improve(s) the antimicrobial effect of 3-hydroxypropyl caprylate.

According to a preferred embodiment, the mixture comprises or consists of 3-hydroxypropyl caprylate and imidazolidinyl urea, preferably wherein the total amounts of 3-hydroxypropyl caprylate and imidazolidinyl urea, respectively, contained in the mixture are such that 3-hydroxypropyl caprylate synergistically improves the antimicrobial effect of imidazolidinyl urea and/or that imidazolidinyl urea synergistically improve(s) the antimicrobial effect of 3-hydroxypropyl caprylate.

According to a preferred embodiment, the mixture comprises or consists of 3-hydroxypropyl caprylate and chlorphenesin, preferably wherein the total amounts of 3-hydroxypropyl caprylate and chlorphenesin, respectively, contained in the mixture are such that 3-hydroxypropyl caprylate synergistically improves the antimicrobial effect of chlorphenesin and/or that chlorphenesin synergistically improve(s) the antimicrobial effect of 3-hydroxypropyl caprylate.

According to a preferred embodiment, the mixture comprises or consists of 3-hydroxypropyl caprylate and disodium EDTA, preferably wherein the total amounts of 3-hydroxypropyl caprylate and disodium EDTA, respectively, contained in the mixture are such that 3-hydroxypropyl caprylate synergistically improves the antimicrobial effect of disodium EDTA and/or that disodium EDTA synergistically improve(s) the antimicrobial effect of 3-hydroxypropyl caprylate.

According to a preferred embodiment, the mixture comprises or consists of 3-hydroxypropyl caprylate and tetrasodium glutamate diacetate (glutamic acid N,N-diacetic tetrasodium salt), preferably wherein the total amounts of 3-hydroxypropyl caprylate and tetrasodium glutamate diacetate (glutamic acid N,N-diacetic tetrasodium salt), respectively, contained in the mixture are such that 3-hydroxypropyl caprylate synergistically improves the antimicrobial effect of tetrasodium glutamate diacetate (glutamic acid N,N-diacetic tetrasodium salt) and/or that tetrasodium glutamate diacetate (glutamic acid N,N-diacetic tetrasodium salt) synergistically improve(s) the antimicrobial effect of 3-hydroxypropyl caprylate.

According to a preferred embodiment, the mixture comprises or consists of 3-hydroxypropyl caprylate and trisodium ethylenediamine disuccinate, preferably wherein the total amounts of 3-hydroxypropyl caprylate and trisodium ethylenediamine disuccinate, respectively, contained in the mixture are such that 3-hydroxypropyl caprylate synergistically improves the antimicrobial effect of trisodium ethylenediamine disuccinate and/or that trisodium ethylenediamine disuccinate synergistically improve(s) the antimicrobial effect of 3-hydroxypropyl caprylate.

According to a preferred embodiment, the mixture comprises or consists of 3-hydroxypropyl caprylate and methylglycine N,N-diacetic trisodium salt, preferably wherein the total amounts of 3-hydroxypropyl caprylate and methylglycine N,N-diacetic trisodium salt, respectively, contained in the mixture are such that 3-hydroxypropyl caprylate synergistically improves the antimicrobial effect of methylglycine N, N-diacetic trisodium salt and/or that methylglycine N,N-diacetic trisodium salt synergistically improve(s) the antimicrobial effect of 3-hydroxypropyl caprylate.

According to a preferred embodiment, the mixture comprises or consists of 3-hydroxypropyl caprylate and glucoheptonic sodium salt, preferably wherein the total amounts of 3-hydroxypropyl caprylate and glucoheptonic sodium salt, respectively, contained in the mixture are such that 3-hydroxypropyl caprylate synergistically improves the antimicrobial effect of glucoheptonic sodium salt and/or that glucoheptonic sodium salt synergistically improve(s) the antimicrobial effect of 3-hydroxypropyl caprylate.

According to a preferred embodiment, the mixture comprises or consists of 3-hydroxypropyl caprylate and ethanoldiglycinic disodium salt, preferably wherein the total amounts of 3-hydroxypropyl caprylate and ethanoldiglycinic disodium salt, respectively, contained in the mixture are such that 3-hydroxypropyl caprylate synergistically improves the antimicrobial effect of ethanoldiglycinic disodium salt and/or that ethanoldiglycinic disodium salt synergistically improve(s) the antimicrobial effect of 3-hydroxypropyl caprylate.

According to a preferred embodiment, the mixture comprises or consists of 3-hydroxypropyl caprylate and sodium gluconate, preferably wherein the total amounts of 3-hydroxypropyl caprylate and sodium gluconate, respectively, contained in the mixture are such that 3-hydroxypropyl caprylate synergistically improves the antimicrobial effect of sodium gluconate and/or that sodium gluconate synergistically improve(s) the antimicrobial effect of 3-hydroxypropyl caprylate.

According to a preferred embodiment, the mixture comprises or consists of 3-hydroxypropyl caprylate and tetrasodium iminodisuccinate, preferably wherein the total amounts of 3-hydroxypropyl caprylate and tetrasodium iminodisuccinate, respectively, contained in the mixture are such that 3-hydroxypropyl caprylate synergistically improves the antimicrobial effect of tetrasodium iminodisuccinate and/or that tetrasodium iminodisuccinate synergistically improve(s) the antimicrobial effect of 3-hydroxypropyl caprylate.

According to a preferred embodiment, the mixture comprises or consists of 3-hydroxypropyl caprylate and trisodium salt of methylglycine diacetic acid, preferably wherein the total amounts of 3-hydroxypropyl caprylate and trisodium salt of methylglycine diacetic acid, respectively, contained in the mixture are such that 3-hydroxypropyl caprylate synergistically improves the antimicrobial effect of trisodium salt of methylglycine diacetic acid and/or that trisodium salt of methylglycine diacetic acid synergistically improve(s) the antimicrobial effect of 3-hydroxypropyl caprylate.

According to a preferred embodiment, the mixture comprises or consists of 3-hydroxypropyl caprylate and polyaspartic acid sodium salt, preferably wherein the total amounts of 3-hydroxypropyl caprylate and polyaspartic acid sodium salt, respectively, contained in the mixture are such that 3-hydroxypropyl caprylate synergistically improves the antimicrobial effect of polyaspartic acid sodium salt and/or that polyaspartic acid sodium salt synergistically improve(s) the antimicrobial effect of 3-hydroxypropyl caprylate.

According to a preferred embodiment, the mixture comprises or consists of 3-hydroxypropyl caprylate and phenoxyisopropanol, preferably wherein the total amounts of 3-hydroxypropyl caprylate and phenoxyisopropanol, respectively, contained in the mixture are such that 3-hydroxypropyl caprylate synergistically improves the antimicrobial effect of phenoxyisopropanol and/or that phenoxyisopropanol synergistically improve(s) the antimicrobial effect of 3-hydroxypropyl caprylate.

According to a preferred embodiment, the mixture comprises or consists of 3-hydroxypropyl caprylate and chlorhexidine, preferably wherein the total amounts of 3-hydroxypropyl caprylate and chlorhexidine, respectively, contained in the mixture are such that 3-hydroxypropyl caprylate synergistically improves the antimicrobial effect of chlorhexidine and/or that chlorhexidine synergistically improve(s) the antimicrobial effect of 3-hydroxypropyl caprylate.

According to a preferred embodiment, the mixture comprises or consists of 3-hydroxypropyl caprylate and chlorhexidine digluconate, preferably wherein the total amounts of 3-hydroxypropyl caprylate and chlorhexidine digluconate, respectively, contained in the mixture are such that 3-hydroxypropyl caprylate synergistically improves the antimicrobial effect of chlorhexidine digluconate and/or that chlorhexidine digluconate synergistically improve(s) the antimicrobial effect of 3-hydroxypropyl caprylate.

According to a preferred embodiment, the mixture comprises or consists of 3-hydroxypropyl caprylate and triclocarban, preferably wherein the total amounts of 3-hydroxypropyl caprylate and triclocarban, respectively, contained in the mixture are such that 3-hydroxypropyl caprylate synergistically improves the antimicrobial effect of triclocarban and/or that triclocarban synergistically improve(s) the antimicrobial effect of 3-hydroxypropyl caprylate.

According to a preferred embodiment, the mixture comprises or consists of 3-hydroxypropyl caprylate and triclosan, preferably wherein the total amounts of 3-hydroxypropyl caprylate and triclosan, respectively, contained in the mixture are such that 3-hydroxypropyl caprylate synergistically improves the antimicrobial effect of triclosan and/or that triclosan synergistically improve(s) the antimicrobial effect of 3-hydroxypropyl caprylate.

According to a preferred embodiment, the mixture comprises or consists of 3-hydroxypropyl caprylate and O-phenylphenol, preferably wherein the total amounts of 3-hydroxypropyl caprylate and O-phenylphenol, respectively, contained in the mixture are such that 3-hydroxypropyl caprylate synergistically improves the antimicrobial effect of O-phenylphenol and/or that O-phenylphenol synergistically improve(s) the antimicrobial effect of 3-hydroxypropyl caprylate.

According to a preferred embodiment, the mixture comprises or consists of 3-hydroxypropyl caprylate and 4-hydroxybenzoic acid, preferably wherein the total amounts of 3-hydroxypropyl caprylate and 4-hydroxybenzoic acid, respectively, contained in the mixture are such that 3-hydroxypropyl caprylate synergistically improves the antimicrobial effect of 4-hydroxybenzoic acid and/or that 4-hydroxybenzoic acid synergistically improve(s) the antimicrobial effect of 3-hydroxypropyl caprylate.

According to a preferred embodiment, the mixture comprises or consists of 3-hydroxypropyl caprylate and TEA-salicylate, preferably wherein the total amounts of 3-hydroxypropyl caprylate and TEA-salicylate, respectively, contained in the mixture are such that 3-hydroxypropyl caprylate synergistically improves the antimicrobial effect of TEA-salicylate and/or that TEA-salicylate synergistically improve(s) the antimicrobial effect of 3-hydroxypropyl caprylate.

According to a preferred embodiment, the mixture comprises or consists of 3-hydroxypropyl caprylate and benzyl salicylate, preferably wherein the total amounts of 3-hydroxypropyl caprylate and benzyl salicylate, respectively, contained in the mixture are such that 3-hydroxypropyl caprylate synergistically improves the antimicrobial effect of benzyl salicylate and/or that benzyl salicylate synergistically improve(s) the antimicrobial effect of 3-hydroxypropyl caprylate.

According to a preferred embodiment, the mixture comprises or consists of 3-hydroxypropyl caprylate and hexamidine, preferably wherein the total amounts of 3-hydroxypropyl caprylate and hexamidine, respectively, contained in the mixture are such that 3-hydroxypropyl caprylate synergistically improves the antimicrobial effect of hexamidine and/or that hexamidine synergistically improve(s) the antimicrobial effect of 3-hydroxypropyl caprylate.

According to a preferred embodiment, the mixture comprises or consists of 3-hydroxypropyl caprylate and hexamidine diisethionate, preferably wherein the total amounts of 3-hydroxypropyl caprylate and hexamidine diisethionate, respectively, contained in the mixture are such that 3-hydroxypropyl caprylate synergistically improves the antimicrobial effect of hexamidine diisethionate and/or that hexamidine diisethionate synergistically improve(s) the antimicrobial effect of 3-hydroxypropyl caprylate.

According to a preferred embodiment, the mixture comprises or consists of 3-hydroxypropyl caprylate and dichlorobenzyl alcohol, preferably wherein the total amounts of 3-hydroxypropyl caprylate and dichlorobenzyl alcohol, respectively, contained in the mixture are such that 3-hydroxypropyl caprylate synergistically improves the antimicrobial effect of dichlorobenzyl alcohol and/or that dichlorobenzyl alcohol synergistically improve(s) the antimicrobial effect of 3-hydroxypropyl caprylate.

According to a preferred embodiment, the mixture comprises or consists of 3-hydroxypropyl caprylate and ethyl benzoate, preferably wherein the total amounts of 3-hydroxypropyl caprylate and ethyl benzoate, respectively, contained in the mixture are such that 3-hydroxypropyl caprylate synergistically improves the antimicrobial effect of ethyl benzoate and/or that ethyl benzoate synergistically improve(s) the antimicrobial effect of 3-hydroxypropyl caprylate.

According to a preferred embodiment, the mixture comprises or consists of 3-hydroxypropyl caprylate and sodium paraben, preferably wherein the total amounts of 3-hydroxypropyl caprylate and sodium paraben, respectively, contained in the mixture are such that 3-hydroxypropyl caprylate synergistically improves the antimicrobial effect of sodium paraben and/or that sodium paraben synergistically improve(s) the antimicrobial effect of 3-hydroxypropyl caprylate.

According to a preferred embodiment, the mixture comprises or consists of 3-hydroxypropyl caprylate and isothiazolinones, preferably wherein the total amounts of 3-hydroxypropyl caprylate and isothiazolinones, respectively, contained in the mixture are such that 3-hydroxypropyl caprylate synergistically improves the antimicrobial effect of isothiazolinones and/or that isothiazolinones synergistically improve(s) the antimicrobial effect of 3-hydroxypropyl caprylate.

According to a preferred embodiment, the mixture comprises or consists of 3-hydroxypropyl caprylate and p-chloro-m-cresol, preferably wherein the total amounts of 3-hydroxypropyl caprylate and p-chloro-m-cresol, respectively, contained in the mixture are such that 3-hydroxypropyl caprylate synergistically improves the antimicrobial effect of p-chloro-m-cresol and/or that p-chloro-m-cresol synergistically improve(s) the antimicrobial effect of 3-hydroxypropyl caprylate.

According to a preferred embodiment, the mixture comprises or consists of 3-hydroxypropyl caprylate and cetrimonium chloride, preferably wherein the total amounts of 3-hydroxypropyl caprylate and cetrimonium chloride, respectively, contained in the mixture are such that 3-hydroxypropyl caprylate synergistically improves the antimicrobial effect of cetrimonium chloride and/or that cetrimonium chloride synergistically improve(s) the antimicrobial effect of 3-hydroxypropyl caprylate.

According to a preferred embodiment, the mixture comprises or consists of 3-hydroxypropyl caprylate and cetrimonium bromide, preferably wherein the total amounts of 3-hydroxypropyl caprylate and cetrimonium bromide, respectively, contained in the mixture are such that 3-hydroxypropyl caprylate synergistically improves the antimicrobial effect of cetrimonium bromide and/or that cetrimonium bromide synergistically improve(s) the antimicrobial effect of 3-hydroxypropyl caprylate.

According to a preferred embodiment, the mixture comprises or consists of 3-hydroxypropyl caprylate and benzethonium chloride, preferably wherein the total amounts of 3-hydroxypropyl caprylate and benzethonium chloride, respectively, contained in the mixture are such that 3-hydroxypropyl caprylate synergistically improves the antimicrobial effect of benzethonium chloride and/or that benzethonium chloride synergistically improve(s) the antimicrobial effect of 3-hydroxypropyl caprylate.

According to a preferred embodiment, the mixture comprises or consists of 3-hydroxypropyl caprylate and alkyltrimonium bromide (alkyltrimethylammonium bromide, Cetrimide mix), preferably wherein the total amounts of 3-hydroxypropyl caprylate and alkyltrimonium bromide (alkyltrimethylammonium bromide, Cetrimide mix), respectively, contained in the mixture are such that 3-hydroxypropyl caprylate synergistically improves the antimicrobial effect of alkyltrimonium bromide (alkyltrimethylammonium bromide, Cetrimide mix) and/or that alkyltrimonium bromide (alkyltrimethylammonium bromide, Cetrimide mix) synergistically improve(s) the antimicrobial effect of 3-hydroxypropyl caprylate.

According to a preferred embodiment, the mixture comprises or consists of 3-hydroxypropyl caprylate and alkyltrimonium chloride, preferably wherein the total amounts of 3-hydroxypropyl caprylate and alkyltrimonium chloride, respectively, contained in the mixture are such that 3-hydroxypropyl caprylate synergistically improves the antimicrobial effect of alkyltrimonium chloride and/or that alkyltrimonium chloride synergistically improve(s) the antimicrobial effect of 3-hydroxypropyl caprylate.

According to a preferred embodiment, the mixture comprises or consists of 3-hydroxypropyl caprylate and benzalkonium chloride (mixture of alkylbenzyldimethylammonium chlorides), preferably wherein the total amounts of 3-hydroxypropyl caprylate and benzalkonium chloride (mixture of alkylbenzyldimethylammonium chlorides), respectively, contained in the mixture are such that 3-hydroxypropyl caprylate synergistically improves the antimicrobial effect of benzalkonium chloride (mixture of alkylbenzyldimethylammonium chlorides) and/or that benzalkonium chloride (mixture of alkylbenzyldimethylammonium chlorides) synergistically improve(s) the antimicrobial effect of 3-hydroxypropyl caprylate.

According to a preferred embodiment, the mixture comprises or consists of 3-hydroxypropyl caprylate and benzalkonium bromide, preferably wherein the total amounts of 3-hydroxypropyl caprylate and benzalkonium bromide, respectively, contained in the mixture are such that 3-hydroxypropyl caprylate synergistically improves the antimicrobial effect of benzalkonium bromide and/or that benzalkonium bromide synergistically improve(s) the antimicrobial effect of 3-hydroxypropyl caprylate.

According to a preferred embodiment, the mixture comprises or consists of 3-hydroxypropyl caprylate and myristamidopropyl PG-dimonium chloride phosphate, preferably wherein the total amounts of 3-hydroxypropyl caprylate and myristamidopropyl PG-dimonium chloride phosphate, respectively, contained in the mixture are such that 3-hydroxypropyl caprylate synergistically improves the antimicrobial effect of myristamidopropyl PG-dimonium chloride phosphate and/or that myristamidopropyl PG-dimonium chloride phosphate synergistically improve(s) the antimicrobial effect of 3-hydroxypropyl caprylate.

According to a preferred embodiment, the mixture comprises or consists of 3-hydroxypropyl caprylate and guar hydroxypropyltrimonium chloride, preferably wherein the total amounts of 3-hydroxypropyl caprylate and guar hydroxypropyltrimonium chloride, respectively, contained in the mixture are such that 3-hydroxypropyl caprylate synergistically improves the antimicrobial effect of guar hydroxypropyltrimonium chloride and/or that guar hydroxypropyltrimonium chloride synergistically improve(s) the antimicrobial effect of 3-hydroxypropyl caprylate.

According to a preferred embodiment, the mixture comprises or consists of 3-hydroxypropyl caprylate and hydroxypropyl guar hydroxypropyltrimonium chloride, preferably wherein the total amounts of 3-hydroxypropyl caprylate and hydroxypropyl guar hydroxypropyltrimonium chloride, respectively, contained in the mixture are such that 3-hydroxypropyl caprylate synergistically improves the antimicrobial effect of hydroxypropyl guar hydroxypropyltrimonium chloride and/or that hydroxypropyl guar hydroxypropyltrimonium chloride synergistically improve(s) the antimicrobial effect of 3-hydroxypropyl caprylate.

According to a preferred embodiment, the mixture comprises or consists of 3-hydroxypropyl caprylate and polyquaternium-6, preferably wherein the total amounts of 3-hydroxypropyl caprylate and polyquaternium-6, respectively, contained in the mixture are such that 3-hydroxypropyl caprylate synergistically improves the antimicrobial effect of polyquaternium-6 and/or that polyquaternium-6 synergistically improve(s) the antimicrobial effect of 3-hydroxypropyl caprylate.

According to a preferred embodiment, the mixture comprises or consists of 3-hydroxypropyl caprylate and polyquaternium-7, preferably wherein the total amounts of 3-hydroxypropyl caprylate and polyquaternium-7, respectively, contained in the mixture are such that 3-hydroxypropyl caprylate synergistically improves the antimicrobial effect of polyquaternium-7 and/or that polyquaternium-7 synergistically improve(s) the antimicrobial effect of 3-hydroxypropyl caprylate.

According to a preferred embodiment, the mixture comprises or consists of 3-hydroxypropyl caprylate and polyquaternium-10, preferably wherein the total amounts of 3-hydroxypropyl caprylate and polyquaternium-10, respectively, contained in the mixture are such that 3-hydroxypropyl caprylate synergistically improves the antimicrobial effect of polyquaternium-10 and/or that polyquaternium-10 synergistically improve(s) the antimicrobial effect of 3-hydroxypropyl caprylate.

According to a preferred embodiment, the mixture comprises or consists of 3-hydroxypropyl caprylate and polyquaternium-11, preferably wherein the total amounts of 3-hydroxypropyl caprylate and polyquaternium-11, respectively, contained in the mixture are such that 3-hydroxypropyl caprylate synergistically improves the antimicrobial effect of polyquaternium-11 and/or that polyquaternium-11 synergistically improve(s) the antimicrobial effect of 3-hydroxypropyl caprylate.

According to a preferred embodiment, the mixture comprises or consists of 3-hydroxypropyl caprylate and polyquaternium-12, preferably wherein the total amounts of 3-hydroxypropyl caprylate and polyquaternium-12, respectively, contained in the mixture are such that 3-hydroxypropyl caprylate synergistically improves the antimicrobial effect of polyquaternium-12 and/or that polyquaternium-12 synergistically improve(s) the antimicrobial effect of 3-hydroxypropyl caprylate.

According to a preferred embodiment, the mixture comprises or consists of 3-hydroxypropyl caprylate and polyquaternium-15, preferably wherein the total amounts of 3-hydroxypropyl caprylate and polyquaternium-15, respectively, contained in the mixture are such that 3-hydroxypropyl caprylate synergistically improves the antimicrobial effect of polyquaternium-15 and/or that polyquaternium-15 synergistically improve(s) the antimicrobial effect of 3-hydroxypropyl caprylate.

According to a preferred embodiment, the mixture comprises or consists of 3-hydroxypropyl caprylate and methylheptylglycerin, preferably wherein the total amounts of 3-hydroxypropyl caprylate and methylheptylglycerin, respectively, contained in the mixture are such that 3-hydroxypropyl caprylate synergistically improves the antimicrobial effect of methylheptylglycerin and/or that methylheptylglycerin synergistically improve(s) the antimicrobial effect of 3-hydroxypropyl caprylate.

According to a preferred embodiment, the mixture comprises or consists of 3-hydroxypropyl caprylate and sodium caproyl/lauroyl lactylate, preferably wherein the total amounts of 3-hydroxypropyl caprylate and sodium caproyl/lauroyl lactylate, respectively, contained in the mixture are such that 3-hydroxypropyl caprylate synergistically improves the antimicrobial effect of sodium caproyl/lauroyl lactylate and/or that sodium caproyl/lauroyl lactylate synergistically improve(s) the antimicrobial effect of 3-hydroxypropyl caprylate.

According to a preferred embodiment, the mixture comprises or consists of 3-hydroxypropyl caprylate and polyglyceryl-10 laurate, preferably wherein the total amounts of 3-hydroxypropyl caprylate and polyglyceryl-10 laurate, respectively, contained in the mixture are such that 3-hydroxypropyl caprylate synergistically improves the antimicrobial effect of polyglyceryl-10 laurate and/or that polyglyceryl-10 laurate synergistically improve(s) the antimicrobial effect of 3-hydroxypropyl caprylate.

According to a preferred embodiment, the mixture comprises or consists of 3-hydroxypropyl caprylate and glyceryl caprate, preferably wherein the total amounts of 3-hydroxypropyl caprylate and glyceryl caprate, respectively, contained in the mixture are such that 3-hydroxypropyl caprylate synergistically improves the antimicrobial effect of glyceryl caprate and/or that glyceryl caprate synergistically improve(s) the antimicrobial effect of 3-hydroxypropyl caprylate.

According to a preferred embodiment, the mixture comprises or consists of 3-hydroxypropyl caprylate and polyglyceryl-2 caprate, preferably wherein the total amounts of 3-hydroxypropyl caprylate and polyglyceryl-2 caprate, respectively, contained in the mixture are such that 3-hydroxypropyl caprylate synergistically improves the antimicrobial effect of polyglyceryl-2 caprate and/or that polyglyceryl-2 caprate synergistically improve(s) the antimicrobial effect of 3-hydroxypropyl caprylate.

According to a preferred embodiment, the mixture comprises or consists of 3-hydroxypropyl caprylate and raspberry ketone, preferably wherein the total amounts of 3-hydroxypropyl caprylate and raspberry ketone, respectively, contained in the mixture are such that 3-hydroxypropyl caprylate synergistically improves the antimicrobial effect of raspberry ketone and/or that raspberry ketone synergistically improve(s) the antimicrobial effect of 3-hydroxypropyl caprylate.

According to a preferred embodiment, the mixture comprises or consists of 3-hydroxypropyl caprylate and raspberry ketone methyl ether, preferably wherein the total amounts of 3-hydroxypropyl caprylate and raspberry ketone methyl ether, respectively, contained in the mixture are such that 3-hydroxypropyl caprylate synergistically improves the antimicrobial effect of and raspberry ketone methyl ether and/or that and raspberry ketone methyl ether synergistically improve(s) the antimicrobial effect of 3-hydroxypropyl caprylate.

According to a preferred embodiment, the mixture comprises or consists of 3-hydroxypropyl caprylate and caprylyl caprylate, preferably wherein the total amounts of 3-hydroxypropyl caprylate and caprylyl caprylate, respectively, contained in the mixture are such that 3-hydroxypropyl caprylate synergistically improves the antimicrobial effect of caprylyl caprylate and/or that caprylyl caprylate synergistically improve(s) the antimicrobial effect of 3-hydroxypropyl caprylate.

According to a preferred embodiment, the mixture comprises or consists of 3-hydroxypropyl caprylate and coco-caprylate/caprate, preferably wherein the total amounts of 3-hydroxypropyl caprylate and coco-caprylate/caprate, respectively, contained in the mixture are such that 3-hydroxypropyl caprylate synergistically improves the antimicrobial effect of coco-caprylate/caprate and/or that coco-caprylate/caprate synergistically improve(s) the antimicrobial effect of 3-hydroxypropyl caprylate.

According to a preferred embodiment, the mixture comprises or consists of 3-hydroxypropyl caprylate and PEG-8 caprylate, preferably wherein the total amounts of 3-hydroxypropyl caprylate and PEG-8 caprylate, respectively, contained in the mixture are such that 3-hydroxypropyl caprylate synergistically improves the antimicrobial effect of PEG-8 caprylate and/or that PEG-8 caprylate synergistically improve(s) the antimicrobial effect of 3-hydroxypropyl caprylate.

According to a preferred embodiment, the mixture comprises or consists of 3-hydroxypropyl caprylate and dipropylene glycol caprylate, preferably wherein the total amounts of 3-hydroxypropyl caprylate and dipropylene glycol caprylate, respectively, contained in the mixture are such that 3-hydroxypropyl caprylate synergistically improves the antimicrobial effect of dipropylene glycol caprylate and/or that dipropylene glycol caprylate synergistically improve(s) the antimicrobial effect of 3-hydroxypropyl caprylate.

According to a preferred embodiment, the mixture comprises or consists of 3-hydroxypropyl caprylate and propylene glycol caprylate, preferably wherein the total amounts of 3-hydroxypropyl caprylate and propylene glycol caprylate, respectively, contained in the mixture are such that 3-hydroxypropyl caprylate synergistically improves the antimicrobial effect of propylene glycol caprylate and/or that propylene glycol caprylate synergistically improve(s) the antimicrobial effect of 3-hydroxypropyl caprylate.

According to a preferred embodiment, the mixture comprises or consists of 3-hydroxypropyl caprylate and pyridoxine dicaprylate, preferably wherein the total amounts of 3-hydroxypropyl caprylate and pyridoxine dicaprylate, respectively, contained in the mixture are such that 3-hydroxypropyl caprylate synergistically improves the antimicrobial effect of pyridoxine dicaprylate and/or that pyridoxine dicaprylate synergistically improve(s) the antimicrobial effect of 3-hydroxypropyl caprylate.

According to a preferred embodiment, the mixture comprises or consists of 3-hydroxypropyl caprylate and glyceryl caprylate/caprate, preferably wherein the total amounts of 3-hydroxypropyl caprylate and glyceryl caprylate/caprate, respectively, contained in the mixture are such that 3-hydroxypropyl caprylate synergistically improves the antimicrobial effect of glyceryl caprylate/caprate and/or that glyceryl caprylate/caprate synergistically improve(s) the antimicrobial effect of 3-hydroxypropyl caprylate.

According to a preferred embodiment, the mixture comprises or consists of 3-hydroxypropyl caprylate and methyl glucose caprylate/caprate, preferably wherein the total amounts of 3-hydroxypropyl caprylate and methyl glucose caprylate/caprate, respectively, contained in the mixture are such that 3-hydroxypropyl caprylate synergistically improves the antimicrobial effect of methyl glucose caprylate/caprate and/or that methyl glucose caprylate/caprate synergistically improve(s) the antimicrobial effect of 3-hydroxypropyl caprylate.

According to a preferred embodiment, the mixture comprises or consists of 3-hydroxypropyl caprylate and polyglyceryl-3 caprate/caprylate/succinate, preferably wherein the total amounts of 3-hydroxypropyl caprylate and polyglyceryl-3 caprate/caprylate/succinate, respectively, contained in the mixture are such that 3-hydroxypropyl caprylate synergistically improves the antimicrobial effect of polyglyceryl-3 caprate/caprylate/succinate and/or that polyglyceryl-3 caprate/caprylate/succinate synergistically improve(s) the antimicrobial effect of 3-hydroxypropyl caprylate.

According to a preferred embodiment, the mixture comprises or consists of 3-hydroxypropyl caprylate and polyglyceryl-2 caprylate, preferably wherein the total amounts of 3-hydroxypropyl caprylate and polyglyceryl-2 caprylate, respectively, contained in the mixture are such that 3-hydroxypropyl caprylate synergistically improves the antimicrobial effect of polyglyceryl-2 caprylate and/or that polyglyceryl-2 caprylate synergistically improve(s) the antimicrobial effect of 3-hydroxypropyl caprylate.

According to a preferred embodiment, the mixture comprises or consists of 3-hydroxypropyl caprylate and polyglyceryl-3 caprylate, preferably wherein the total amounts of 3-hydroxypropyl caprylate and polyglyceryl-3 caprylate, respectively, contained in the mixture are such that 3-hydroxypropyl caprylate synergistically improves the antimicrobial effect of polyglyceryl-3 caprylate and/or that polyglyceryl-3 caprylate synergistically improve(s) the antimicrobial effect of 3-hydroxypropyl caprylate.

According to a preferred embodiment, the mixture comprises or consists of 3-hydroxypropyl caprylate and polyglyceryl-4 caprylate, preferably wherein the total amounts of 3-hydroxypropyl caprylate and polyglyceryl-4 caprylate, respectively, contained in the mixture are such that 3-hydroxypropyl caprylate synergistically improves the antimicrobial effect of polyglyceryl-4 caprylate and/or that polyglyceryl-4 caprylate synergistically improve(s) the antimicrobial effect of 3-hydroxypropyl caprylate.

According to a preferred embodiment, the mixture comprises or consists of 3-hydroxypropyl caprylate and polyglyceryl-6 caprylate, preferably wherein the total amounts of 3-hydroxypropyl caprylate and polyglyceryl-6 caprylate, respectively, contained in the mixture are such that 3-hydroxypropyl caprylate synergistically improves the antimicrobial effect of polyglyceryl-6 caprylate and/or that polyglyceryl-6 caprylate synergistically improve(s) the antimicrobial effect of 3-hydroxypropyl caprylate.

According to a preferred embodiment, the mixture comprises or consists of 3-hydroxypropyl caprylate and polyglyceryl-10 caprylate, preferably wherein the total amounts of 3-hydroxypropyl caprylate and polyglyceryl-10 caprylate, respectively, contained in the mixture are such that 3-hydroxypropyl caprylate synergistically improves the antimicrobial effect of polyglyceryl-10 caprylate and/or that polyglyceryl-10 caprylate synergistically improve(s) the antimicrobial effect of 3-hydroxypropyl caprylate.

According to a preferred embodiment, the mixture comprises or consists of 3-hydroxypropyl caprylate and polyglyceryl-4 caprylate/caprate, preferably wherein the total amounts of 3-hydroxypropyl caprylate and polyglyceryl-4 caprylate/caprate, respectively, contained in the mixture are such that 3-hydroxypropyl caprylate synergistically improves the antimicrobial effect of polyglyceryl-4 caprylate/caprate and/or that polyglyceryl-4 caprylate/caprate synergistically improve(s) the antimicrobial effect of 3-hydroxypropyl caprylate.

According to a preferred embodiment, the mixture comprises or consists of 3-hydroxypropyl caprylate and polyglyceryl-6 caprylate/caprate, preferably wherein the total amounts of 3-hydroxypropyl caprylate and polyglyceryl-6 caprylate/caprate, respectively, contained in the mixture are such that 3-hydroxypropyl caprylate synergistically improves the antimicrobial effect of polyglyceryl-6 caprylate/caprate and/or that polyglyceryl-6 caprylate/caprate synergistically improve(s) the antimicrobial effect of 3-hydroxypropyl caprylate.

According to a preferred embodiment, the mixture comprises or consists of 3-hydroxypropyl caprylate and polyglyceryl-10 caprylate/caprate, preferably wherein the total amounts of 3-hydroxypropyl caprylate and polyglyceryl-10 caprylate/caprate, respectively, contained in the mixture are such that 3-hydroxypropyl caprylate synergistically improves the antimicrobial effect of polyglyceryl-10 caprylate/caprate and/or that polyglyceryl-10 caprylate/caprate synergistically improve(s) the antimicrobial effect of 3-hydroxypropyl caprylate.

According to a preferred embodiment, the mixture comprises or consists of 3-hydroxypropyl caprylate and propanediol dicaprylate/caprate, preferably wherein the total amounts of 3-hydroxypropyl caprylate and propanediol dicaprylate/caprate, respectively, contained in the mixture are such that 3-hydroxypropyl caprylate synergistically improves the antimicrobial effect of propanediol dicaprylate/caprate and/or that propanediol dicaprylate/caprate synergistically improve(s) the antimicrobial effect of 3-hydroxypropyl caprylate.

According to a preferred embodiment, the mixture comprises or consists of 3-hydroxypropyl caprylate and xylityl caprate/caprylate, preferably wherein the total amounts of 3-hydroxypropyl caprylate and xylityl caprate/caprylate, respectively, contained in the mixture are such that 3-hydroxypropyl caprylate synergistically improves the antimicrobial effect of xylityl caprate/caprylate and/or that xylityl caprate/caprylate synergistically improve(s) the antimicrobial effect of 3-hydroxypropyl caprylate.

According to a preferred embodiment, the mixture comprises or consists of 3-hydroxypropyl caprylate and xylityl caprylate, preferably wherein the total amounts of 3-hydroxypropyl caprylate and xylityl caprylate, respectively, contained in the mixture are such that 3-hydroxypropyl caprylate synergistically improves the antimicrobial effect of xylityl caprylate and/or that xylityl caprylate synergistically improve(s) the antimicrobial effect of 3-hydroxypropyl caprylate.

According to a preferred embodiment, the mixture comprises or consists of 3-hydroxypropyl caprylate and xylityl sesquicaprylate, preferably wherein the total amounts of 3-hydroxypropyl caprylate and xylityl sesquicaprylate, respectively, contained in the mixture are such that 3-hydroxypropyl caprylate synergistically improves the antimicrobial effect of xylityl sesquicaprylate and/or that xylityl sesquicaprylate synergistically improve(s) the antimicrobial effect of 3-hydroxypropyl caprylate.

According to a preferred embodiment, the mixture comprises or consists of 3-hydroxypropyl caprylate and hydroxyethoxyphenyl butanone, preferably wherein the total amounts of 3-hydroxypropyl caprylate and hydroxyethoxyphenyl butanone, respectively, contained in the mixture are such that 3-hydroxypropyl caprylate synergistically improves the antimicrobial effect of hydroxyethoxyphenyl butanone and/or that hydroxyethoxyphenyl butanone synergistically improve(s) the antimicrobial effect of 3-hydroxypropyl caprylate.

According to a preferred embodiment, the mixture comprises or consists of 3-hydroxypropyl undecylenate and piroctone olamine, preferably wherein the total amounts of 3-hydroxypropyl undecylenate and piroctone olamine, respectively, contained in the mixture are such that 3-hydroxypropyl undecylenate synergistically improves the antimicrobial effect of piroctone olamine and/or that piroctone olamine synergistically improve(s) the antimicrobial effect of 3-hydroxypropyl undecylenate.

According to a preferred embodiment, the mixture comprises or consists of 3-hydroxypropyl undecylenate and climbazole, preferably wherein the total amounts of 3-hydroxypropyl undecylenate and climbazole, respectively, contained in the mixture are such that 3-hydroxypropyl undecylenate synergistically improves the antimicrobial effect of climbazole and/or that climbazole synergistically improve(s) the antimicrobial effect of 3-hydroxypropyl undecylenate.

According to a preferred embodiment, the mixture comprises or consists of 3-hydroxypropyl undecylenate and zinc pyrithione, preferably wherein the total amounts of 3-hydroxypropyl undecylenate and zinc pyrithione, respectively, contained in the mixture are such that 3-hydroxypropyl undecylenate synergistically improves the antimicrobial effect of zinc pyrithione and/or that zinc pyrithione synergistically improve(s) the antimicrobial effect of 3-hydroxypropyl undecylenate.

According to a preferred embodiment, the mixture comprises or consists of 3-hydroxypropyl undecylenate and salicylic acid (and/or salts thereof), preferably wherein the total amounts of 3-hydroxypropyl undecylenate and salicylic acid (and/or salts thereof), respectively, contained in the mixture are such that 3-hydroxypropyl undecylenate synergistically improves the antimicrobial effect of salicylic acid (and/or salts thereof) and/or that salicylic acid (and/or salts thereof) synergistically improve(s) the antimicrobial effect of 3-hydroxypropyl undecylenate.

According to a preferred embodiment, the mixture comprises or consists of 3-hydroxypropyl undecylenate and decylene glycol, preferably wherein the total amounts of 3-hydroxypropyl undecylenate and decylene glycol, respectively, contained in the mixture are such that 3-hydroxypropyl undecylenate synergistically improves the antimicrobial effect of decylene glycol and/or that decylene glycol synergistically improve(s) the antimicrobial effect of 3-hydroxypropyl undecylenate.

According to a preferred embodiment, the mixture comprises or consists of 3-hydroxypropyl undecylenate and o-cymen-5-ol, preferably wherein the total amounts of 3-hydroxypropyl undecylenate and o-cymen-5-ol, respectively, contained in the mixture are such that 3-hydroxypropyl undecylenate synergistically improves the antimicrobial effect of o-cymen-5-ol and/or that o-cymen-5-ol synergistically improve(s) the antimicrobial effect of 3-hydroxypropyl undecylenate.

According to a preferred embodiment, the mixture comprises or consists of 3-hydroxypropyl undecylenate and selenium sulfide, preferably wherein the total amounts of 3-hydroxypropyl undecylenate and selenium sulfide, respectively, contained in the mixture are such that 3-hydroxypropyl undecylenate synergistically improves the antimicrobial effect of selenium sulfide and/or that selenium sulfide synergistically improve(s) the antimicrobial effect of 3-hydroxypropyl undecylenate.

According to a preferred embodiment, the mixture comprises or consists of 3-hydroxypropyl undecylenate and coal tar, preferably wherein the total amounts of 3-hydroxypropyl undecylenate and coal tar, respectively, contained in the mixture are such that 3-hydroxypropyl undecylenate synergistically improves the antimicrobial effect of coal tar and/or that coal tar synergistically improve(s) the antimicrobial effect of 3-hydroxypropyl undecylenate.

According to a preferred embodiment, the mixture comprises or consists of 3-hydroxypropyl undecylenate and sulphur, preferably wherein the total amounts of 3-hydroxypropyl undecylenate and sulphur, respectively, contained in the mixture are such that 3-hydroxypropyl undecylenate synergistically improves the antimicrobial effect of sulphur and/or that sulphur synergistically improve(s) the antimicrobial effect of 3-hydroxypropyl undecylenate.

According to a preferred embodiment, the mixture comprises or consists of 3-hydroxypropyl undecylenate and ketoconazole, preferably wherein the total amounts of 3-hydroxypropyl undecylenate and ketoconazole, respectively, contained in the mixture are such that 3-hydroxypropyl undecylenate synergistically improves the antimicrobial effect of ketoconazole and/or that ketoconazole synergistically improve(s) the antimicrobial effect of 3-hydroxypropyl undecylenate.

According to a preferred embodiment, the mixture comprises or consists of 3-hydroxypropyl undecylenate and benzoic acid (and/or salts thereof, preferably sodium benzoate), preferably wherein the total amounts of 3-hydroxypropyl undecylenate and benzoic acid (and/or salts thereof, preferably sodium benzoate), respectively, contained in the mixture are such that 3-hydroxypropyl undecylenate synergistically improves the antimicrobial effect of benzoic acid (and/or salts thereof, preferably sodium benzoate) and/or that benzoic acid (and/or salts thereof, preferably sodium benzoate) synergistically improve(s) the antimicrobial effect of 3-hydroxypropyl undecylenate.

According to a preferred embodiment, the mixture comprises or consists of 3-hydroxypropyl undecylenate and sorbic acid (and/or salts thereof, preferably potassium sorbate), preferably wherein the total amounts of 3-hydroxypropyl undecylenate and sorbic acid (and/or salts thereof, preferably potassium sorbate), respectively, contained in the mixture are such that 3-hydroxypropyl undecylenate synergistically improves the antimicrobial effect of sorbic acid (and/or salts thereof, preferably potassium sorbate) and/or that sorbic acid (and/or salts thereof, preferably potassium sorbate) synergistically improve(s) the antimicrobial effect of 3-hydroxypropyl undecylenate.

According to a preferred embodiment, the mixture comprises or consists of 3-hydroxypropyl undecylenate and lactic acid (and/or salts thereof), preferably wherein the total amounts of 3-hydroxypropyl undecylenate and lactic acid (and/or salts thereof), respectively, contained in the mixture are such that 3-hydroxypropyl undecylenate synergistically improves the antimicrobial effect of lactic acid (and/or salts thereof) and/or that lactic acid (and/or salts thereof) synergistically improve(s) the antimicrobial effect of 3-hydroxypropyl undecylenate.

According to a preferred embodiment, the mixture comprises or consists of 3-hydroxypropyl undecylenate and dehydroacetic acid (and/or salts thereof, preferably sodium dehydroacetate), preferably wherein the total amounts of 3-hydroxypropyl undecylenate and dehydroacetic acid (and/or salts thereof, preferably sodium dehydroacetate), respectively, contained in the mixture are such that 3-hydroxypropyl undecylenate synergistically improves the antimicrobial effect of dehydroacetic acid (and/or salts thereof, preferably sodium dehydroacetate) and/or that dehydroacetic acid (and/or salts thereof, preferably sodium dehydroacetate) synergistically improve(s) the antimicrobial effect of 3-hydroxypropyl undecylenate.

According to a preferred embodiment, the mixture comprises or consists of 3-hydroxypropyl undecylenate and undecylenic acid (and/or salts thereof), preferably wherein the total amounts of 3-hydroxypropyl undecylenate and undecylenic acid (and/or salts thereof), respectively, contained in the mixture are such that 3-hydroxypropyl undecylenate synergistically improves the antimicrobial effect of undecylenic acid (and/or salts thereof) and/or that undecylenic acid (and/or salts thereof) synergistically improve(s) the antimicrobial effect of 3-hydroxypropyl undecylenate.

According to a preferred embodiment, the mixture comprises or consists of 3-hydroxypropyl undecylenate and levulinic acid (and/or salts thereof, preferably sodium levulinate), preferably wherein the total amounts of 3-hydroxypropyl undecylenate and levulinic acid (and/or salts thereof, preferably sodium levulinate), respectively, contained in the mixture are such that 3-hydroxypropyl undecylenate synergistically improves the antimicrobial effect of levulinic acid (and/or salts thereof, preferably sodium levulinate) and/or that levulinic acid (and/or salts thereof, preferably sodium levulinate) synergistically improve(s) the antimicrobial effect of 3-hydroxypropyl undecylenate.

According to a preferred embodiment, the mixture comprises or consists of 3-hydroxypropyl undecylenate and anisic acid (and/or salts thereof), preferably wherein the total amounts of 3-hydroxypropyl undecylenate and anisic acid (and/or salts thereof), respectively, contained in the mixture are such that 3-hydroxypropyl undecylenate synergistically improves the antimicrobial effect of anisic acid (and/or salts thereof) and/or that anisic acid (and/or salts thereof) synergistically improve(s) the antimicrobial effect of 3-hydroxypropyl undecylenate.

According to a preferred embodiment, the mixture comprises or consists of 3-hydroxypropyl undecylenate and phytic acid (and/or salts thereof, preferably sodium phytate), preferably wherein the total amounts of 3-hydroxypropyl undecylenate and phytic acid (and/or salts thereof, preferably sodium phytate), respectively, contained in the mixture are such that 3-hydroxypropyl undecylenate synergistically improves the antimicrobial effect of phytic acid (and/or salts thereof, preferably sodium phytate) and/or that phytic acid (and/or salts thereof, preferably sodium phytate) synergistically improve(s) the antimicrobial effect of 3-hydroxypropyl undecylenate.

According to a preferred embodiment, the mixture comprises or consists of 3-hydroxypropyl undecylenate and itaconic acid (and/or salts thereof), preferably wherein the total amounts of 3-hydroxypropyl undecylenate and itaconic acid (and/or salts thereof), respectively, contained in the mixture are such that 3-hydroxypropyl undecylenate synergistically improves the antimicrobial effect of itaconic acid (and/or salts thereof) and/or that itaconic acid (and/or salts thereof) synergistically improve(s) the antimicrobial effect of 3-hydroxypropyl undecylenate.

According to a preferred embodiment, the mixture comprises or consists of 3-hydroxypropyl undecylenate and MEA undecylenate, preferably wherein the total amounts of 3-hydroxypropyl undecylenate and MEA undecylenate, respectively, contained in the mixture are such that 3-hydroxypropyl undecylenate synergistically improves the antimicrobial effect of MEA undecylenate and/or that MEA undecylenate synergistically improve(s) the antimicrobial effect of 3-hydroxypropyl undecylenate.

According to a preferred embodiment, the mixture comprises or consists of 3-hydroxypropyl undecylenate and butyloctanoic acid (and/or salts thereof), preferably wherein the total amounts of 3-hydroxypropyl undecylenate and butyloctanoic acid (and/or salts thereof), respectively, contained in the mixture are such that 3-hydroxypropyl undecylenate synergistically improves the antimicrobial effect of butyloctanoic acid (and/or salts thereof) and/or that butyloctanoic acid (and/or salts thereof) synergistically improve(s) the antimicrobial effect of 3-hydroxypropyl undecylenate.

According to a preferred embodiment, the mixture comprises or consists of 3-hydroxypropyl undecylenate and citric acid (and/or salts thereof), preferably wherein the total amounts of 3-hydroxypropyl undecylenate and citric acid (and/or salts thereof), respectively, contained in the mixture are such that 3-hydroxypropyl undecylenate synergistically improves the antimicrobial effect of citric acid (and/or salts thereof) and/or that citric acid (and/or salts thereof) synergistically improve(s) the antimicrobial effect of 3-hydroxypropyl undecylenate.

According to a preferred embodiment, the mixture comprises or consists of 3-hydroxypropyl undecylenate and hydroxyacetophenone, preferably wherein the total amounts of 3-hydroxypropyl undecylenate and hydroxyacetophenone, respectively, contained in the mixture are such that 3-hydroxypropyl undecylenate synergistically improves the antimicrobial effect of hydroxyacetophenone and/or that hydroxyacetophenone synergistically improve(s) the antimicrobial effect of 3-hydroxypropyl undecylenate.

According to a preferred embodiment, the mixture comprises or consists of 3-hydroxypropyl undecylenate and ethylhexylglycerin, preferably wherein the total amounts of 3-hydroxypropyl undecylenate and ethylhexylglycerin, respectively, contained in the mixture are such that 3-hydroxypropyl undecylenate synergistically improves the antimicrobial effect of ethylhexylglycerin and/or that ethylhexylglycerin synergistically improve(s) the antimicrobial effect of 3-hydroxypropyl undecylenate.

According to a preferred embodiment, the mixture comprises or consists of 3-hydroxypropyl undecylenate and caprylhydroxamic acid (and/or salts thereof), preferably wherein the total amounts of 3-hydroxypropyl undecylenate and caprylhydroxamic acid (and/or salts thereof), respectively, contained in the mixture are such that 3-hydroxypropyl undecylenate synergistically improves the antimicrobial effect of caprylhydroxamic acid (and/or salts thereof) and/or that caprylhydroxamic acid (and/or salts thereof) synergistically improve(s) the antimicrobial effect of 3-hydroxypropyl undecylenate.

According to a preferred embodiment, the mixture comprises or consists of 3-hydroxypropyl undecylenate and undecylenoyl glycine, preferably wherein the total amounts of 3-hydroxypropyl undecylenate and undecylenoyl glycine, respectively, contained in the mixture are such that 3-hydroxypropyl undecylenate synergistically improves the antimicrobial effect of undecylenoyl glycine and/or that undecylenoyl glycine synergistically improve(s) the antimicrobial effect of 3-hydroxypropyl undecylenate.

According to a preferred embodiment, the mixture comprises or consists of 3-hydroxypropyl undecylenate and capryloyl glycine, preferably wherein the total amounts of 3-hydroxypropyl undecylenate and capryloyl glycine, respectively, contained in the mixture are such that 3-hydroxypropyl undecylenate synergistically improves the antimicrobial effect of capryloyl glycine and/or that capryloyl glycine synergistically improve(s) the antimicrobial effect of 3-hydroxypropyl undecylenate.

According to a preferred embodiment, the mixture comprises or consists of 3-hydroxypropyl undecylenate and polyglyceryl-3 caprylate, preferably wherein the total amounts of 3-hydroxypropyl undecylenate and polyglyceryl-3 caprylate, respectively, contained in the mixture are such that 3-hydroxypropyl undecylenate synergistically improves the antimicrobial effect of polyglyceryl-3 caprylate and/or that polyglyceryl-3 caprylate synergistically improve(s) the antimicrobial effect of 3-hydroxypropyl undecylenate.

According to a preferred embodiment, the mixture comprises or consists of 3-hydroxypropyl undecylenate and glyceryl undecylenate, preferably wherein the total amounts of 3-hydroxypropyl undecylenate and glyceryl undecylenate, respectively, contained in the mixture are such that 3-hydroxypropyl undecylenate synergistically improves the antimicrobial effect of glyceryl undecylenate and/or that glyceryl undecylenate synergistically improve(s) the antimicrobial effect of 3-hydroxypropyl undecylenate.

According to a preferred embodiment, the mixture comprises or consists of 3-hydroxypropyl undecylenate and glyceryl caprylate, preferably wherein the total amounts of 3-hydroxypropyl undecylenate and glyceryl caprylate, respectively, contained in the mixture are such that 3-hydroxypropyl undecylenate synergistically improves the antimicrobial effect of glyceryl caprylate and/or that glyceryl caprylate synergistically improve(s) the antimicrobial effect of 3-hydroxypropyl undecylenate.

According to a preferred embodiment, the mixture comprises or consists of 3-hydroxypropyl undecylenate and sorbitan caprylate, preferably wherein the total amounts of 3-hydroxypropyl undecylenate and sorbitan caprylate, respectively, contained in the mixture are such that 3-hydroxypropyl undecylenate synergistically improves the antimicrobial effect of sorbitan caprylate and/or that sorbitan caprylate synergistically improve(s) the antimicrobial effect of 3-hydroxypropyl undecylenate.

According to a preferred embodiment, the mixture comprises or consists of 3-hydroxypropyl undecylenate and glyceryl laurate, preferably wherein the total amounts of 3-hydroxypropyl undecylenate and glyceryl laurate, respectively, contained in the mixture are such that 3-hydroxypropyl undecylenate synergistically improves the antimicrobial effect of glyceryl laurate and/or that glyceryl laurate synergistically improve(s) the antimicrobial effect of 3-hydroxypropyl undecylenate.

According to a preferred embodiment, the mixture comprises or consists of 3-hydroxypropyl undecylenate and phenethyl alcohol, preferably wherein the total amounts of 3-hydroxypropyl undecylenate and phenethyl alcohol, respectively, contained in the mixture are such that 3-hydroxypropyl undecylenate synergistically improves the antimicrobial effect of phenethyl alcohol and/or that phenethyl alcohol synergistically improve(s) the antimicrobial effect of 3-hydroxypropyl undecylenate.

According to a preferred embodiment, the mixture comprises or consists of 3-hydroxypropyl undecylenate and tropolones, preferably wherein the total amounts of 3-hydroxypropyl undecylenate and tropolones, respectively, contained in the mixture are such that 3-hydroxypropyl undecylenate synergistically improves the antimicrobial effect of tropolones and/or that tropolones synergistically improve(s) the antimicrobial effect of 3-hydroxypropyl undecylenate.

According to a preferred embodiment, the mixture comprises or consists of 3-hydroxypropyl undecylenate and 1,2-propanediol, preferably wherein the total amounts of 3-hydroxypropyl undecylenate and 1,2-propanediol, respectively, contained in the mixture are such that 3-hydroxypropyl undecylenate synergistically improves the antimicrobial effect of 1,2-propanediol and/or that 1,2-propanediol synergistically improve(s) the antimicrobial effect of 3-hydroxypropyl undecylenate.

According to a preferred embodiment, the mixture comprises or consists of 3-hydroxypropyl undecylenate and 1,2-butanediol, preferably wherein the total amounts of 3-hydroxypropyl undecylenate and 1,2-butanediol, respectively, contained in the mixture are such that 3-hydroxypropyl undecylenate synergistically improves the antimicrobial effect of 1,2-butanediol and/or that 1,2-butanediol synergistically improve(s) the antimicrobial effect of 3-hydroxypropyl undecylenate.

According to a preferred embodiment, the mixture comprises or consists of 3-hydroxypropyl undecylenate and 1,2-pentanediol, preferably wherein the total amounts of 3-hydroxypropyl undecylenate and 1,2-pentanediol, respectively, contained in the mixture are such that 3-hydroxypropyl undecylenate synergistically improves the antimicrobial effect of 1,2-pentanediol and/or that 1,2-pentanediol synergistically improve(s) the antimicrobial effect of 3-hydroxypropyl undecylenate.

According to a preferred embodiment, the mixture comprises or consists of 3-hydroxypropyl undecylenate and 1,2-hexanediol, preferably wherein the total amounts of 3-hydroxypropyl undecylenate and 1,2-hexanediol, respectively, contained in the mixture are such that 3-hydroxypropyl undecylenate synergistically improves the antimicrobial effect of 1,2-hexanediol and/or that 1,2-hexanediol synergistically improve(s) the antimicrobial effect of 3-hydroxypropyl undecylenate.

According to a preferred embodiment, the mixture comprises or consists of 3-hydroxypropyl undecylenate and 1,2-heptanediol, preferably wherein the total amounts of 3-hydroxypropyl undecylenate and 1,2-heptanediol, respectively, contained in the mixture are such that 3-hydroxypropyl undecylenate synergistically improves the antimicrobial effect of 1,2-heptanediol and/or that 1,2-heptanediol synergistically improve(s) the antimicrobial effect of 3-hydroxypropyl undecylenate.

According to a preferred embodiment, the mixture comprises or consists of 3-hydroxypropyl undecylenate and 1,2-octanediol, preferably wherein the total amounts of 3-hydroxypropyl undecylenate and 1,2-octanediol, respectively, contained in the mixture are such that 3-hydroxypropyl undecylenate synergistically improves the antimicrobial effect of 1,2-octanediol and/or that 1,2-octanediol synergistically improve(s) the antimicrobial effect of 3-hydroxypropyl undecylenate.

According to a preferred embodiment, the mixture comprises or consists of 3-hydroxypropyl undecylenate and 1,2-nonanediol, preferably wherein the total amounts of 3-hydroxypropyl undecylenate and 1,2-nonanediol, respectively, contained in the mixture are such that 3-hydroxypropyl undecylenate synergistically improves the antimicrobial effect of 1,2-nonanediol and/or that 1,2-nonanediol synergistically improve(s) the antimicrobial effect of 3-hydroxypropyl undecylenate.

According to a preferred embodiment, the mixture comprises or consists of 3-hydroxypropyl undecylenate and 1,2-decanediol, preferably wherein the total amounts of 3-hydroxypropyl undecylenate and 1,2-decanediol, respectively, contained in the mixture are such that 3-hydroxypropyl undecylenate synergistically improves the antimicrobial effect of 1,2-decanediol and/or that 1,2-decanediol synergistically improve(s) the antimicrobial effect of 3-hydroxypropyl undecylenate.

According to a preferred embodiment, the mixture comprises or consists of 3-hydroxypropyl undecylenate and 1,2-undecanediol, preferably wherein the total amounts of 3-hydroxypropyl undecylenate and 1,2-undecanediol, respectively, contained in the mixture are such that 3-hydroxypropyl undecylenate synergistically improves the antimicrobial effect of 1,2-undecanediol and/or that 1,2-undecanediol synergistically improve(s) the antimicrobial effect of 3-hydroxypropyl undecylenate.

According to a preferred embodiment, the mixture comprises or consists of 3-hydroxypropyl undecylenate and 1,2-dodecanediol, preferably wherein the total amounts of 3-hydroxypropyl undecylenate and 1,2-dodecanediol, respectively, contained in the mixture are such that 3-hydroxypropyl undecylenate synergistically improves the antimicrobial effect of 1,2-dodecanediol and/or that 1,2-dodecanediol synergistically improve(s) the antimicrobial effect of 3-hydroxypropyl undecylenate.

According to a preferred embodiment, the mixture comprises or consists of 3-hydroxypropyl undecylenate and 1,2-tridecanediol, preferably wherein the total amounts of 3-hydroxypropyl undecylenate and 1,2-tridecanediol, respectively, contained in the mixture are such that 3-hydroxypropyl undecylenate synergistically improves the antimicrobial effect of 1,2-tridecanediol and/or that 1,2-tridecanediol synergistically improve(s) the antimicrobial effect of 3-hydroxypropyl undecylenate.

According to a preferred embodiment, the mixture comprises or consists of 3-hydroxypropyl undecylenate and 2,3-pentanediol, preferably wherein the total amounts of 3-hydroxypropyl undecylenate and 2,3-pentanediol, respectively, contained in the mixture are such that 3-hydroxypropyl undecylenate synergistically improves the antimicrobial effect of 2,3-pentanediol and/or that 2,3-pentanediol synergistically improve(s) the antimicrobial effect of 3-hydroxypropyl undecylenate.

According to a preferred embodiment, the mixture comprises or consists of 3-hydroxypropyl undecylenate and 2,3-hexanediol, preferably wherein the total amounts of 3-hydroxypropyl undecylenate and 2,3-hexanediol, respectively, contained in the mixture are such that 3-hydroxypropyl undecylenate synergistically improves the antimicrobial effect of 2,3-hexanediol and/or that 2,3-hexanediol synergistically improve(s) the antimicrobial effect of 3-hydroxypropyl undecylenate.

According to a preferred embodiment, the mixture comprises or consists of 3-hydroxypropyl undecylenate and 2,3-heptanediol, preferably wherein the total amounts of 3-hydroxypropyl undecylenate and 2,3-heptanediol, respectively, contained in the mixture are such that 3-hydroxypropyl undecylenate synergistically improves the antimicrobial effect of 2,3-heptanediol and/or that 2,3-heptanediol synergistically improve(s) the antimicrobial effect of 3-hydroxypropyl undecylenate.

According to a preferred embodiment, the mixture comprises or consists of 3-hydroxypropyl undecylenate and 2,3-octanediol, preferably wherein the total amounts of 3-hydroxypropyl undecylenate and 2,3-octanediol, respectively, contained in the mixture are such that 3-hydroxypropyl undecylenate synergistically improves the antimicrobial effect of 2,3-octanediol and/or that 2,3-octanediol synergistically improve(s) the antimicrobial effect of 3-hydroxypropyl undecylenate.

According to a preferred embodiment, the mixture comprises or consists of 3-hydroxypropyl undecylenate and 2,3-nonanediol, preferably wherein the total amounts of 3-hydroxypropyl undecylenate and 2,3-nonanediol, respectively, contained in the mixture are such that 3-hydroxypropyl undecylenate synergistically improves the antimicrobial effect of 2,3-nonanediol and/or that 2,3-nonanediol synergistically improve(s) the antimicrobial effect of 3-hydroxypropyl undecylenate.

According to a preferred embodiment, the mixture comprises or consists of 3-hydroxypropyl undecylenate and 2,3-decanediol, preferably wherein the total amounts of 3-hydroxypropyl undecylenate and 2,3-decanediol, respectively, contained in the mixture are such that 3-hydroxypropyl undecylenate synergistically improves the antimicrobial effect of 2,3-decanediol and/or that 2,3-decanediol synergistically improve(s) the antimicrobial effect of 3-hydroxypropyl undecylenate.

According to a preferred embodiment, the mixture comprises or consists of 3-hydroxypropyl undecylenate and 2,3-undecanediol, preferably wherein the total amounts of 3-hydroxypropyl undecylenate and 2,3-undecanediol, respectively, contained in the mixture are such that 3-hydroxypropyl undecylenate synergistically improves the antimicrobial effect of 2,3-undecanediol and/or that 2,3-undecanediol synergistically improve(s) the antimicrobial effect of 3-hydroxypropyl undecylenate.

According to a preferred embodiment, the mixture comprises or consists of 3-hydroxypropyl undecylenate and 2,3-dodecanediol, preferably wherein the total amounts of 3-hydroxypropyl undecylenate and 2,3-dodecanediol, respectively, contained in the mixture are such that 3-hydroxypropyl undecylenate synergistically improves the antimicrobial effect of 2,3-dodecanediol and/or that 2,3-dodecanediol synergistically improve(s) the antimicrobial effect of 3-hydroxypropyl undecylenate.

According to a preferred embodiment, the mixture comprises or consists of 3-hydroxypropyl undecylenate and 2,3-tridecanediol, preferably wherein the total amounts of 3-hydroxypropyl undecylenate and 2,3-tridecanediol, respectively, contained in the mixture are such that 3-hydroxypropyl undecylenate synergistically improves the antimicrobial effect of 2,3-tridecanediol and/or that 2,3-tridecanediol synergistically improve(s) the antimicrobial effect of 3-hydroxypropyl undecylenate.

According to a preferred embodiment, the mixture comprises or consists of 3-hydroxypropyl undecylenate and 1,3-propanediol, preferably wherein the total amounts of 3-hydroxypropyl undecylenate and 1,3-propanediol, respectively, contained in the mixture are such that 3-hydroxypropyl undecylenate synergistically improves the antimicrobial effect of 1,3-propanediol and/or that 1,3-propanediol synergistically improve(s) the antimicrobial effect of 3-hydroxypropyl undecylenate.

According to a preferred embodiment, the mixture comprises or consists of 3-hydroxypropyl undecylenate and 1,3-butanediol, preferably wherein the total amounts of 3-hydroxypropyl undecylenate and 1,3-butanediol, respectively, contained in the mixture are such that 3-hydroxypropyl undecylenate synergistically improves the antimicrobial effect of 1,3-butanediol and/or that 1,3-butanediol synergistically improve(s) the antimicrobial effect of 3-hydroxypropyl undecylenate.

According to a preferred embodiment, the mixture comprises or consists of 3-hydroxypropyl undecylenate and 2-methyl-2,4-pentanediol, preferably wherein the total amounts of 3-hydroxypropyl undecylenate and 2-methyl-2,4-pentanediol, respectively, contained in the mixture are such that 3-hydroxypropyl undecylenate synergistically improves the antimicrobial effect of 2-methyl-2,4-pentanediol and/or that 2-methyl-2,4-pentanediol synergistically improve(s) the antimicrobial effect of 3-hydroxypropyl undecylenate.

According to a preferred embodiment, the mixture comprises or consists of 3-hydroxypropyl undecylenate and caprylyl glycol, preferably wherein the total amounts of 3-hydroxypropyl undecylenate and caprylyl glycol, respectively, contained in the mixture are such that 3-hydroxypropyl undecylenate synergistically improves the antimicrobial effect of caprylyl glycol and/or that caprylyl glycol synergistically improve(s) the antimicrobial effect of 3-hydroxypropyl undecylenate.

According to a preferred embodiment, the mixture comprises or consists of 3-hydroxypropyl undecylenate and farnesol, preferably wherein the total amounts of 3-hydroxypropyl undecylenate and farnesol, respectively, contained in the mixture are such that 3-hydroxypropyl undecylenate synergistically improves the antimicrobial effect of farnesol and/or that farnesol synergistically improve(s) the antimicrobial effect of 3-hydroxypropyl undecylenate.

According to a preferred embodiment, the mixture comprises or consists of 3-hydroxypropyl undecylenate and bisabolol, preferably wherein the total amounts of 3-hydroxypropyl undecylenate and bisabolol, respectively, contained in the mixture are such that 3-hydroxypropyl undecylenate synergistically improves the antimicrobial effect of bisabolol and/or that bisabolol synergistically improve(s) the antimicrobial effect of 3-hydroxypropyl undecylenate.

According to a preferred embodiment, the mixture comprises or consists of 3-hydroxypropyl undecylenate and nerolidol, preferably wherein the total amounts of 3-hydroxypropyl undecylenate and nerolidol, respectively, contained in the mixture are such that 3-hydroxypropyl undecylenate synergistically improves the antimicrobial effect of nerolidol and/or that nerolidol synergistically improve(s) the antimicrobial effect of 3-hydroxypropyl undecylenate.

According to a preferred embodiment, the mixture comprises or consists of 3-hydroxypropyl undecylenate and carvacrol, preferably wherein the total amounts of 3-hydroxypropyl undecylenate and carvacrol, respectively, contained in the mixture are such that 3-hydroxypropyl undecylenate synergistically improves the antimicrobial effect of carvacrol and/or that carvacrol synergistically improve(s) the antimicrobial effect of 3-hydroxypropyl undecylenate.

According to a preferred embodiment, the mixture comprises or consists of 3-hydroxypropyl undecylenate and thymol, preferably wherein the total amounts of 3-hydroxypropyl undecylenate and thymol, respectively, contained in the mixture are such that 3-hydroxypropyl undecylenate synergistically improves the antimicrobial effect of thymol and/or that thymol synergistically improve(s) the antimicrobial effect of 3-hydroxypropyl undecylenate.

According to a preferred embodiment, the mixture comprises or consists of 3-hydroxypropyl undecylenate and phenylpropanol, preferably wherein the total amounts of 3-hydroxypropyl undecylenate and phenylpropanol, respectively, contained in the mixture are such that 3-hydroxypropyl undecylenate synergistically improves the antimicrobial effect of phenylpropanol and/or that phenylpropanol synergistically improve(s) the antimicrobial effect of 3-hydroxypropyl undecylenate.

According to a preferred embodiment, the mixture comprises or consists of 3-hydroxypropyl undecylenate and methylbenzyl alcohol, preferably wherein the total amounts of 3-hydroxypropyl undecylenate and methylbenzyl alcohol, respectively, contained in the mixture are such that 3-hydroxypropyl undecylenate synergistically improves the antimicrobial effect of methylbenzyl alcohol and/or that methylbenzyl alcohol synergistically improve(s) the antimicrobial effect of 3-hydroxypropyl undecylenate.

According to a preferred embodiment, the mixture comprises or consists of 3-hydroxypropyl undecylenate and 2-benzylheptanol, preferably wherein the total amounts of 3-hydroxypropyl undecylenate and 2-benzylheptanol, respectively, contained in the mixture are such that 3-hydroxypropyl undecylenate synergistically improves the antimicrobial effect of 2-benzylheptanol and/or that 2-benzylheptanol synergistically improve(s) the antimicrobial effect of 3-hydroxypropyl undecylenate.

According to a preferred embodiment, the mixture comprises or consists of 3-hydroxypropyl undecylenate and 2-methyl 5-cyclohexylpentanol, preferably wherein the total amounts of 3-hydroxypropyl undecylenate and 2-methyl 5-cyclohexylpentanol, respectively, contained in the mixture are such that 3-hydroxypropyl undecylenate synergistically improves the antimicrobial effect of 2-methyl 5-cyclohexylpentanol and/or that 2-methyl 5-cyclohexylpentanol synergistically improve(s) the antimicrobial effect of 3-hydroxypropyl undecylenate.

According to a preferred embodiment, the mixture comprises or consists of 3-hydroxypropyl undecylenate and dimethyl phenylbutanol, preferably wherein the total amounts of 3-hydroxypropyl undecylenate and dimethyl phenylbutanol, respectively, contained in the mixture are such that 3-hydroxypropyl undecylenate synergistically improves the antimicrobial effect of dimethyl phenylbutanol and/or that dimethyl phenylbutanol synergistically improve(s) the antimicrobial effect of 3-hydroxypropyl undecylenate.

According to a preferred embodiment, the mixture comprises or consists of 3-hydroxypropyl undecylenate and PCA ethyl cocoyl arginate, preferably wherein the total amounts of 3-hydroxypropyl undecylenate and PCA ethyl cocoyl arginate, respectively, contained in the mixture are such that 3-hydroxypropyl undecylenate synergistically improves the antimicrobial effect of PCA ethyl cocoyl arginate and/or that PCA ethyl cocoyl arginate synergistically improve(s) the antimicrobial effect of 3-hydroxypropyl undecylenate.

According to a preferred embodiment, the mixture comprises or consists of 3-hydroxypropyl undecylenate and benzyl alcohol, preferably wherein the total amounts of 3-hydroxypropyl undecylenate and benzyl alcohol, respectively, contained in the mixture are such that 3-hydroxypropyl undecylenate synergistically improves the antimicrobial effect of benzyl alcohol and/or that benzyl alcohol synergistically improve(s) the antimicrobial effect of 3-hydroxypropyl undecylenate.

According to a preferred embodiment, the mixture comprises or consists of 3-hydroxypropyl undecylenate and phenoxyethanol, preferably wherein the total amounts of 3-hydroxypropyl undecylenate and phenoxyethanol, respectively, contained in the mixture are such that 3-hydroxypropyl undecylenate synergistically improves the antimicrobial effect of phenoxyethanol and/or that phenoxyethanol synergistically improve(s) the antimicrobial effect of 3-hydroxypropyl undecylenate.

According to a preferred embodiment, the mixture comprises or consists of 3-hydroxypropyl undecylenate and iodopropynyl butylcarbamate, preferably wherein the total amounts of 3-hydroxypropyl undecylenate and iodopropynyl butylcarbamate, respectively, contained in the mixture are such that 3-hydroxypropyl undecylenate synergistically improves the antimicrobial effect of iodopropynyl butylcarbamate and/or that iodopropynyl butylcarbamate synergistically improve(s) the antimicrobial effect of 3-hydroxypropyl undecylenate.

According to a preferred embodiment, the mixture comprises or consists of 3-hydroxypropyl undecylenate and hydroxyethoxyphenyl butanol, preferably wherein the total amounts of 3-hydroxypropyl undecylenate and hydroxyethoxyphenyl butanol, respectively, contained in the mixture are such that 3-hydroxypropyl undecylenate synergistically improves the antimicrobial effect of hydroxyethoxyphenyl butanol and/or that hydroxyethoxyphenyl butanol synergistically improve(s) the antimicrobial effect of 3-hydroxypropyl undecylenate.

According to a preferred embodiment, the mixture comprises or consists of 3-hydroxypropyl undecylenate and ethyl lauroyl arginate HCl, preferably wherein the total amounts of 3-hydroxypropyl undecylenate and ethyl lauroyl arginate HCl, respectively, contained in the mixture are such that 3-hydroxypropyl undecylenate synergistically improves the antimicrobial effect of ethyl lauroyl arginate HCl and/or that ethyl lauroyl arginate HCl synergistically improve(s) the antimicrobial effect of 3-hydroxypropyl undecylenate.

According to a preferred embodiment, the mixture comprises or consists of 3-hydroxypropyl undecylenate and methylchloroisothiazolinone, preferably wherein the total amounts of 3-hydroxypropyl undecylenate and methylchloroisothiazolinone, respectively, contained in the mixture are such that 3-hydroxypropyl undecylenate synergistically improves the antimicrobial effect of methylchloroisothiazolinone and/or that methylchloroisothiazolinone synergistically improve(s) the antimicrobial effect of 3-hydroxypropyl undecylenate.

According to a preferred embodiment, the mixture comprises or consists of 3-hydroxypropyl undecylenate and methylisothiazolinone, preferably wherein the total amounts of 3-hydroxypropyl undecylenate and methylisothiazolinone, respectively, contained in the mixture are such that 3-hydroxypropyl undecylenate synergistically improves the antimicrobial effect of methylisothiazolinone and/or that methylisothiazolinone synergistically improve(s) the antimicrobial effect of 3-hydroxypropyl undecylenate.

According to a preferred embodiment, the mixture comprises or consists of 3-hydroxypropyl undecylenate and methylparaben (and/or salts thereof, preferably sodium methylparaben), preferably wherein the total amounts of 3-hydroxypropyl undecylenate and methylparaben (and/or salts thereof, preferably sodium methylparaben), respectively, contained in the mixture are such that 3-hydroxypropyl undecylenate synergistically improves the antimicrobial effect of methylparaben (and/or salts thereof, preferably sodium methylparaben) and/or that methylparaben (and/or salts thereof, preferably sodium methylparaben) synergistically improve(s) the antimicrobial effect of 3-hydroxypropyl undecylenate.

According to a preferred embodiment, the mixture comprises or consists of 3-hydroxypropyl undecylenate and ethylparaben (and/or and salts thereof, preferably sodium ethylparaben), preferably wherein the total amounts of 3-hydroxypropyl undecylenate and ethylparaben (and/or and salts thereof, preferably sodium ethylparaben), respectively, contained in the mixture are such that 3-hydroxypropyl undecylenate synergistically improves the antimicrobial effect of ethylparaben (and/or and salts thereof, preferably sodium ethylparaben) and/or that ethylparaben (and/or and salts thereof, preferably sodium ethylparaben) synergistically improve(s) the antimicrobial effect of 3-hydroxypropyl undecylenate.

According to a preferred embodiment, the mixture comprises or consists of 3-hydroxypropyl undecylenate and propylparaben (and/or salts thereof, preferably sodium propylparaben), preferably wherein the total amounts of 3-hydroxypropyl undecylenate and propylparaben (and/or salts thereof, preferably sodium propylparaben), respectively, contained in the mixture are such that 3-hydroxypropyl undecylenate synergistically improves the antimicrobial effect of propylparaben (and/or salts thereof, preferably sodium propylparaben) and/or that propylparaben (and/or salts thereof, preferably sodium propylparaben) synergistically improve(s) the antimicrobial effect of 3-hydroxypropyl undecylenate.

According to a preferred embodiment, the mixture comprises or consists of 3-hydroxypropyl undecylenate and isopropylparaben (and/or salts thereof), preferably wherein the total amounts of 3-hydroxypropyl undecylenate and isopropylparaben (and/or salts thereof), respectively, contained in the mixture are such that 3-hydroxypropyl undecylenate synergistically improves the antimicrobial effect of isopropylparaben (and/or salts thereof) and/or that isopropylparaben (and/or salts thereof) synergistically improve(s) the antimicrobial effect of 3-hydroxypropyl undecylenate.

According to a preferred embodiment, the mixture comprises or consists of 3-hydroxypropyl undecylenate and butylparaben (and/or salts thereof), preferably wherein the total amounts of 3-hydroxypropyl undecylenate and butylparaben (and/or salts thereof), respectively, contained in the mixture are such that 3-hydroxypropyl undecylenate synergistically improves the antimicrobial effect of butylparaben (and/or salts thereof) and/or that butylparaben (and/or salts thereof) synergistically improve(s) the antimicrobial effect of 3-hydroxypropyl undecylenate.

According to a preferred embodiment, the mixture comprises or consists of 3-hydroxypropyl undecylenate and isobutylparaben (and/or salts thereof), preferably wherein the total amounts of 3-hydroxypropyl undecylenate and isobutylparaben (and/or salts thereof), respectively, contained in the mixture are such that 3-hydroxypropyl undecylenate synergistically improves the antimicrobial effect of isobutylparaben (and/or salts thereof) and/or that isobutylparaben (and/or salts thereof) synergistically improve(s) the antimicrobial effect of 3-hydroxypropyl undecylenate.

According to a preferred embodiment, the mixture comprises or consists of 3-hydroxypropyl undecylenate and DMDM hydantoin, preferably wherein the total amounts of 3-hydroxypropyl undecylenate and DMDM hydantoin, respectively, contained in the mixture are such that 3-hydroxypropyl undecylenate synergistically improves the antimicrobial effect of DMDM hydantoin and/or that DMDM hydantoin synergistically improve(s) the antimicrobial effect of 3-hydroxypropyl undecylenate.

According to a preferred embodiment, the mixture comprises or consists of 3-hydroxypropyl undecylenate and diazolidinyl urea, preferably wherein the total amounts of 3-hydroxypropyl undecylenate and diazolidinyl urea, respectively, contained in the mixture are such that 3-hydroxypropyl undecylenate synergistically improves the antimicrobial effect of diazolidinyl urea and/or that diazolidinyl urea synergistically improve(s) the antimicrobial effect of 3-hydroxypropyl undecylenate.

According to a preferred embodiment, the mixture comprises or consists of 3-hydroxypropyl undecylenate and imidazolidinyl urea, preferably wherein the total amounts of 3-hydroxypropyl undecylenate and imidazolidinyl urea, respectively, contained in the mixture are such that 3-hydroxypropyl undecylenate synergistically improves the antimicrobial effect of imidazolidinyl urea and/or that imidazolidinyl urea synergistically improve(s) the antimicrobial effect of 3-hydroxypropyl undecylenate.

According to a preferred embodiment, the mixture comprises or consists of 3-hydroxypropyl undecylenate and chlorphenesin, preferably wherein the total amounts of 3-hydroxypropyl undecylenate and chlorphenesin, respectively, contained in the mixture are such that 3-hydroxypropyl undecylenate synergistically improves the antimicrobial effect of chlorphenesin and/or that chlorphenesin synergistically improve(s) the antimicrobial effect of 3-hydroxypropyl undecylenate.

According to a preferred embodiment, the mixture comprises or consists of 3-hydroxypropyl undecylenate and disodium EDTA, preferably wherein the total amounts of 3-hydroxypropyl undecylenate and disodium EDTA, respectively, contained in the mixture are such that 3-hydroxypropyl undecylenate synergistically improves the antimicrobial effect of disodium EDTA and/or that disodium EDTA synergistically improve(s) the antimicrobial effect of 3-hydroxypropyl undecylenate.

According to a preferred embodiment, the mixture comprises or consists of 3-hydroxypropyl undecylenate and tetrasodium glutamate diacetate (glutamic acid N,N-diacetic tetrasodium salt), preferably wherein the total amounts of 3-hydroxypropyl undecylenate and tetrasodium glutamate diacetate (glutamic acid N,N-diacetic tetrasodium salt), respectively, contained in the mixture are such that 3-hydroxypropyl undecylenate synergistically improves the antimicrobial effect of tetrasodium glutamate diacetate (glutamic acid N,N-diacetic tetrasodium salt) and/or that tetrasodium glutamate diacetate (glutamic acid N,N-diacetic tetrasodium salt) synergistically improve(s) the antimicrobial effect of 3-hydroxypropyl undecylenate.

According to a preferred embodiment, the mixture comprises or consists of 3-hydroxypropyl undecylenate and trisodium ethylenediamine disuccinate, preferably wherein the total amounts of 3-hydroxypropyl undecylenate and trisodium ethylenediamine disuccinate, respectively, contained in the mixture are such that 3-hydroxypropyl undecylenate synergistically improves the antimicrobial effect of trisodium ethylenediamine disuccinate and/or that trisodium ethylenediamine disuccinate synergistically improve(s) the antimicrobial effect of 3-hydroxypropyl undecylenate.

According to a preferred embodiment, the mixture comprises or consists of 3-hydroxypropyl undecylenate and methylglycine N,N-diacetic trisodium salt, preferably wherein the total amounts of 3-hydroxypropyl undecylenate and methylglycine N,N-diacetic trisodium salt, respectively, contained in the mixture are such that 3-hydroxypropyl undecylenate synergistically improves the antimicrobial effect of methylglycine N, N-diacetic trisodium salt and/or that methylglycine N,N-diacetic trisodium salt synergistically improve(s) the antimicrobial effect of 3-hydroxypropyl undecylenate.

According to a preferred embodiment, the mixture comprises or consists of 3-hydroxypropyl undecylenate and glucoheptonic sodium salt, preferably wherein the total amounts of 3-hydroxypropyl undecylenate and glucoheptonic sodium salt, respectively, contained in the mixture are such that 3-hydroxypropyl undecylenate synergistically improves the antimicrobial effect of glucoheptonic sodium salt and/or that glucoheptonic sodium salt synergistically improve(s) the antimicrobial effect of 3-hydroxypropyl undecylenate.

According to a preferred embodiment, the mixture comprises or consists of 3-hydroxypropyl undecylenate and ethanoldiglycinic disodium salt, preferably wherein the total amounts of 3-hydroxypropyl undecylenate and ethanoldiglycinic disodium salt, respectively, contained in the mixture are such that 3-hydroxypropyl undecylenate synergistically improves the antimicrobial effect of ethanoldiglycinic disodium salt and/or that ethanoldiglycinic disodium salt synergistically improve(s) the antimicrobial effect of 3-hydroxypropyl undecylenate.

According to a preferred embodiment, the mixture comprises or consists of 3-hydroxypropyl undecylenate and sodium gluconate, preferably wherein the total amounts of 3-hydroxypropyl undecylenate and sodium gluconate, respectively, contained in the mixture are such that 3-hydroxypropyl undecylenate synergistically improves the antimicrobial effect of sodium gluconate and/or that sodium gluconate synergistically improve(s) the antimicrobial effect of 3-hydroxypropyl undecylenate.

According to a preferred embodiment, the mixture comprises or consists of 3-hydroxypropyl undecylenate and tetrasodium iminodisuccinate, preferably wherein the total amounts of 3-hydroxypropyl undecylenate and tetrasodium iminodisuccinate, respectively, contained in the mixture are such that 3-hydroxypropyl undecylenate synergistically improves the antimicrobial effect of tetrasodium iminodisuccinate and/or that tetrasodium iminodisuccinate synergistically improve(s) the antimicrobial effect of 3-hydroxypropyl undecylenate.

According to a preferred embodiment, the mixture comprises or consists of 3-hydroxypropyl undecylenate and trisodium salt of methylglycine diacetic acid, preferably wherein the total amounts of 3-hydroxypropyl undecylenate and trisodium salt of methylglycine diacetic acid, respectively, contained in the mixture are such that 3-hydroxypropyl undecylenate synergistically improves the antimicrobial effect of trisodium salt of methylglycine diacetic acid and/or that trisodium salt of methylglycine diacetic acid synergistically improve(s) the antimicrobial effect of 3-hydroxypropyl undecylenate.

According to a preferred embodiment, the mixture comprises or consists of 3-hydroxypropyl undecylenate and polyaspartic acid sodium salt, preferably wherein the total amounts of 3-hydroxypropyl undecylenate and polyaspartic acid sodium salt, respectively, contained in the mixture are such that 3-hydroxypropyl undecylenate synergistically improves the antimicrobial effect of polyaspartic acid sodium salt and/or that polyaspartic acid sodium salt synergistically improve(s) the antimicrobial effect of 3-hydroxypropyl undecylenate.

According to a preferred embodiment, the mixture comprises or consists of 3-hydroxypropyl undecylenate and phenoxyisopropanol, preferably wherein the total amounts of 3-hydroxypropyl undecylenate and phenoxyisopropanol, respectively, contained in the mixture are such that 3-hydroxypropyl undecylenate synergistically improves the antimicrobial effect of phenoxyisopropanol and/or that phenoxyisopropanol synergistically improve(s) the antimicrobial effect of 3-hydroxypropyl undecylenate.

According to a preferred embodiment, the mixture comprises or consists of 3-hydroxypropyl undecylenate and chlorhexidine, preferably wherein the total amounts of 3-hydroxypropyl undecylenate and chlorhexidine, respectively, contained in the mixture are such that 3-hydroxypropyl undecylenate synergistically improves the antimicrobial effect of chlorhexidine and/or that chlorhexidine synergistically improve(s) the antimicrobial effect of 3-hydroxypropyl undecylenate.

According to a preferred embodiment, the mixture comprises or consists of 3-hydroxypropyl undecylenate and chlorhexidine digluconate, preferably wherein the total amounts of 3-hydroxypropyl undecylenate and chlorhexidine digluconate, respectively, contained in the mixture are such that 3-hydroxypropyl undecylenate synergistically improves the antimicrobial effect of chlorhexidine digluconate and/or that chlorhexidine digluconate synergistically improve(s) the antimicrobial effect of 3-hydroxypropyl undecylenate.

According to a preferred embodiment, the mixture comprises or consists of 3-hydroxypropyl undecylenate and triclocarban, preferably wherein the total amounts of 3-hydroxypropyl undecylenate and triclocarban, respectively, contained in the mixture are such that 3-hydroxypropyl undecylenate synergistically improves the antimicrobial effect of triclocarban and/or that triclocarban synergistically improve(s) the antimicrobial effect of 3-hydroxypropyl undecylenate.

According to a preferred embodiment, the mixture comprises or consists of 3-hydroxypropyl undecylenate and triclosan, preferably wherein the total amounts of 3-hydroxypropyl undecylenate and triclosan, respectively, contained in the mixture are such that 3-hydroxypropyl undecylenate synergistically improves the antimicrobial effect of triclosan and/or that triclosan synergistically improve(s) the antimicrobial effect of 3-hydroxypropyl undecylenate.

According to a preferred embodiment, the mixture comprises or consists of 3-hydroxypropyl undecylenate and O-phenylphenol, preferably wherein the total amounts of 3-hydroxypropyl undecylenate and O-phenylphenol, respectively, contained in the mixture are such that 3-hydroxypropyl undecylenate synergistically improves the antimicrobial effect of O-phenylphenol and/or that O-phenylphenol synergistically improve(s) the antimicrobial effect of 3-hydroxypropyl undecylenate.

According to a preferred embodiment, the mixture comprises or consists of 3-hydroxypropyl undecylenate and 4-hydroxybenzoic acid, preferably wherein the total amounts of 3-hydroxypropyl undecylenate and 4-hydroxybenzoic acid, respectively, contained in the mixture are such that 3-hydroxypropyl undecylenate synergistically improves the antimicrobial effect of 4-hydroxybenzoic acid and/or that 4-hydroxybenzoic acid synergistically improve(s) the antimicrobial effect of 3-hydroxypropyl undecylenate.

According to a preferred embodiment, the mixture comprises or consists of 3-hydroxypropyl undecylenate and TEA-salicylate, preferably wherein the total amounts of 3-hydroxypropyl undecylenate and TEA-salicylate, respectively, contained in the mixture are such that 3-hydroxypropyl undecylenate synergistically improves the antimicrobial effect of TEA-salicylate and/or that TEA-salicylate synergistically improve(s) the antimicrobial effect of 3-hydroxypropyl undecylenate.

According to a preferred embodiment, the mixture comprises or consists of 3-hydroxypropyl undecylenate and benzyl salicylate, preferably wherein the total amounts of 3-hydroxypropyl undecylenate and benzyl salicylate, respectively, contained in the mixture are such that 3-hydroxypropyl undecylenate synergistically improves the antimicrobial effect of benzyl salicylate and/or that benzyl salicylate synergistically improve(s) the antimicrobial effect of 3-hydroxypropyl undecylenate.

According to a preferred embodiment, the mixture comprises or consists of 3-hydroxypropyl undecylenate and hexamidine, preferably wherein the total amounts of 3-hydroxypropyl undecylenate and hexamidine, respectively, contained in the mixture are such that 3-hydroxypropyl undecylenate synergistically improves the antimicrobial effect of hexamidine and/or that hexamidine synergistically improve(s) the antimicrobial effect of 3-hydroxypropyl undecylenate.

According to a preferred embodiment, the mixture comprises or consists of 3-hydroxypropyl undecylenate and hexamidine diisethionate, preferably wherein the total amounts of 3-hydroxypropyl undecylenate and hexamidine diisethionate, respectively, contained in the mixture are such that 3-hydroxypropyl undecylenate synergistically improves the antimicrobial effect of hexamidine diisethionate and/or that hexamidine diisethionate synergistically improve(s) the antimicrobial effect of 3-hydroxypropyl undecylenate.

According to a preferred embodiment, the mixture comprises or consists of 3-hydroxypropyl undecylenate and dichlorobenzyl alcohol, preferably wherein the total amounts of 3-hydroxypropyl undecylenate and dichlorobenzyl alcohol, respectively, contained in the mixture are such that 3-hydroxypropyl undecylenate synergistically improves the antimicrobial effect of dichlorobenzyl alcohol and/or that dichlorobenzyl alcohol synergistically improve(s) the antimicrobial effect of 3-hydroxypropyl undecylenate.

According to a preferred embodiment, the mixture comprises or consists of 3-hydroxypropyl undecylenate and ethyl benzoate, preferably wherein the total amounts of 3-hydroxypropyl undecylenate and ethyl benzoate, respectively, contained in the mixture are such that 3-hydroxypropyl undecylenate synergistically improves the antimicrobial effect of ethyl benzoate and/or that ethyl benzoate synergistically improve(s) the antimicrobial effect of 3-hydroxypropyl undecylenate.

According to a preferred embodiment, the mixture comprises or consists of 3-hydroxypropyl undecylenate and sodium paraben, preferably wherein the total amounts of 3-hydroxypropyl undecylenate and sodium paraben, respectively, contained in the mixture are such that 3-hydroxypropyl undecylenate synergistically improves the antimicrobial effect of sodium paraben and/or that sodium paraben synergistically improve(s) the antimicrobial effect of 3-hydroxypropyl undecylenate.

According to a preferred embodiment, the mixture comprises or consists of 3-hydroxypropyl undecylenate and isothiazolinones, preferably wherein the total amounts of 3-hydroxypropyl undecylenate and isothiazolinones, respectively, contained in the mixture are such that 3-hydroxypropyl undecylenate synergistically improves the antimicrobial effect of isothiazolinones and/or that isothiazolinones synergistically improve(s) the antimicrobial effect of 3-hydroxypropyl undecylenate.

According to a preferred embodiment, the mixture comprises or consists of 3-hydroxypropyl undecylenate and p-chloro-m-cresol, preferably wherein the total amounts of 3-hydroxypropyl undecylenate and p-chloro-m-cresol, respectively, contained in the mixture are such that 3-hydroxypropyl undecylenate synergistically improves the antimicrobial effect of p-chloro-m-cresol and/or that p-chloro-m-cresol synergistically improve(s) the antimicrobial effect of 3-hydroxypropyl undecylenate.

According to a preferred embodiment, the mixture comprises or consists of 3-hydroxypropyl undecylenate and cetrimonium chloride, preferably wherein the total amounts of 3-hydroxypropyl undecylenate and cetrimonium chloride, respectively, contained in the mixture are such that 3-hydroxypropyl undecylenate synergistically improves the antimicrobial effect of cetrimonium chloride and/or that cetrimonium chloride synergistically improve(s) the antimicrobial effect of 3-hydroxypropyl undecylenate.

According to a preferred embodiment, the mixture comprises or consists of 3-hydroxypropyl undecylenate and cetrimonium bromide, preferably wherein the total amounts of 3-hydroxypropyl undecylenate and cetrimonium bromide, respectively, contained in the mixture are such that 3-hydroxypropyl undecylenate synergistically improves the antimicrobial effect of cetrimonium bromide and/or that cetrimonium bromide synergistically improve(s) the antimicrobial effect of 3-hydroxypropyl undecylenate.

According to a preferred embodiment, the mixture comprises or consists of 3-hydroxypropyl undecylenate and benzethonium chloride, preferably wherein the total amounts of 3-hydroxypropyl undecylenate and benzethonium chloride, respectively, contained in the mixture are such that 3-hydroxypropyl undecylenate synergistically improves the antimicrobial effect of benzethonium chloride and/or that benzethonium chloride synergistically improve(s) the antimicrobial effect of 3-hydroxypropyl undecylenate.

According to a preferred embodiment, the mixture comprises or consists of 3-hydroxypropyl undecylenate and alkyltrimonium bromide (alkyltrimethylammonium bromide, Cetrimide mix), preferably wherein the total amounts of 3-hydroxypropyl undecylenate and alkyltrimonium bromide (alkyltrimethylammonium bromide, Cetrimide mix), respectively, contained in the mixture are such that 3-hydroxypropyl undecylenate synergistically improves the antimicrobial effect of alkyltrimonium bromide (alkyltrimethylammonium bromide, Cetrimide mix) and/or that alkyltrimonium bromide (alkyltrimethylammonium bromide, Cetrimide mix) synergistically improve(s) the antimicrobial effect of 3-hydroxypropyl undecylenate.

According to a preferred embodiment, the mixture comprises or consists of 3-hydroxypropyl undecylenate and alkyltrimonium chloride, preferably wherein the total amounts of 3-hydroxypropyl undecylenate and alkyltrimonium chloride, respectively, contained in the mixture are such that 3-hydroxypropyl undecylenate synergistically improves the antimicrobial effect of alkyltrimonium chloride and/or that alkyltrimonium chloride synergistically improve(s) the antimicrobial effect of 3-hydroxypropyl undecylenate.

According to a preferred embodiment, the mixture comprises or consists of 3-hydroxypropyl undecylenate and benzalkonium chloride (mixture of alkylbenzyldimethylammonium chlorides), preferably wherein the total amounts of 3-hydroxypropyl undecylenate and benzalkonium chloride (mixture of alkylbenzyldimethylammonium chlorides), respectively, contained in the mixture are such that 3-hydroxypropyl undecylenate synergistically improves the antimicrobial effect of benzalkonium chloride (mixture of alkylbenzyldimethylammonium chlorides) and/or that benzalkonium chloride (mixture of alkylbenzyldimethylammonium chlorides) synergistically improve(s) the antimicrobial effect of 3-hydroxypropyl undecylenate.

According to a preferred embodiment, the mixture comprises or consists of 3-hydroxypropyl undecylenate and benzalkonium bromide, preferably wherein the total amounts of 3-hydroxypropyl undecylenate and benzalkonium bromide, respectively, contained in the mixture are such that 3-hydroxypropyl undecylenate synergistically improves the antimicrobial effect of benzalkonium bromide and/or that benzalkonium bromide synergistically improve(s) the antimicrobial effect of 3-hydroxypropyl undecylenate.

According to a preferred embodiment, the mixture comprises or consists of 3-hydroxypropyl undecylenate and myristamidopropyl PG-dimonium chloride phosphate, preferably wherein the total amounts of 3-hydroxypropyl undecylenate and myristamidopropyl PG-dimonium chloride phosphate, respectively, contained in the mixture are such that 3-hydroxypropyl undecylenate synergistically improves the antimicrobial effect of myristamidopropyl PG-dimonium chloride phosphate and/or that myristamidopropyl PG-dimonium chloride phosphate synergistically improve(s) the antimicrobial effect of 3-hydroxypropyl undecylenate.

According to a preferred embodiment, the mixture comprises or consists of 3-hydroxypropyl undecylenate and guar hydroxypropyltrimonium chloride, preferably wherein the total amounts of 3-hydroxypropyl undecylenate and guar hydroxypropyltrimonium chloride, respectively, contained in the mixture are such that 3-hydroxypropyl undecylenate synergistically improves the antimicrobial effect of guar hydroxypropyltrimonium chloride and/or that guar hydroxypropyltrimonium chloride synergistically improve(s) the antimicrobial effect of 3-hydroxypropyl undecylenate.

According to a preferred embodiment, the mixture comprises or consists of 3-hydroxypropyl undecylenate and hydroxypropyl guar hydroxypropyltrimonium chloride, preferably wherein the total amounts of 3-hydroxypropyl undecylenate and hydroxypropyl guar hydroxypropyltrimonium chloride, respectively, contained in the mixture are such that 3-hydroxypropyl undecylenate synergistically improves the antimicrobial effect of hydroxypropyl guar hydroxypropyltrimonium chloride and/or that hydroxypropyl guar hydroxypropyltrimonium chloride synergistically improve(s) the antimicrobial effect of 3-hydroxypropyl undecylenate.

According to a preferred embodiment, the mixture comprises or consists of 3-hydroxypropyl undecylenate and polyquaternium-6, preferably wherein the total amounts of 3-hydroxypropyl undecylenate and polyquaternium-6, respectively, contained in the mixture are such that 3-hydroxypropyl undecylenate synergistically improves the antimicrobial effect of polyquaternium-6 and/or that polyquaternium-6 synergistically improve(s) the antimicrobial effect of 3-hydroxypropyl undecylenate.

According to a preferred embodiment, the mixture comprises or consists of 3-hydroxypropyl undecylenate and polyquaternium-7, preferably wherein the total amounts of 3-hydroxypropyl undecylenate and polyquaternium-7, respectively, contained in the mixture are such that 3-hydroxypropyl undecylenate synergistically improves the antimicrobial effect of polyquaternium-7 and/or that polyquaternium-7 synergistically improve(s) the antimicrobial effect of 3-hydroxypropyl undecylenate.

According to a preferred embodiment, the mixture comprises or consists of 3-hydroxypropyl undecylenate and polyquaternium-10, preferably wherein the total amounts of 3-hydroxypropyl undecylenate and polyquaternium-10, respectively, contained in the mixture are such that 3-hydroxypropyl undecylenate synergistically improves the antimicrobial effect of polyquaternium-10 and/or that polyquaternium-10 synergistically improve(s) the antimicrobial effect of 3-hydroxypropyl undecylenate.

According to a preferred embodiment, the mixture comprises or consists of 3-hydroxypropyl undecylenate and polyquaternium-11, preferably wherein the total amounts of 3-hydroxypropyl undecylenate and polyquaternium-11, respectively, contained in the mixture are such that 3-hydroxypropyl undecylenate synergistically improves the antimicrobial effect of polyquaternium-11 and/or that polyquaternium-11 synergistically improve(s) the antimicrobial effect of 3-hydroxypropyl undecylenate.

According to a preferred embodiment, the mixture comprises or consists of 3-hydroxypropyl undecylenate and polyquaternium-12, preferably wherein the total amounts of 3-hydroxypropyl undecylenate and polyquaternium-12, respectively, contained in the mixture are such that 3-hydroxypropyl undecylenate synergistically improves the antimicrobial effect of polyquaternium-12 and/or that polyquaternium-12 synergistically improve(s) the antimicrobial effect of 3-hydroxypropyl undecylenate.

According to a preferred embodiment, the mixture comprises or consists of 3-hydroxypropyl undecylenate and polyquaternium-15, preferably wherein the total amounts of 3-hydroxypropyl undecylenate and polyquaternium-15, respectively, contained in the mixture are such that 3-hydroxypropyl undecylenate synergistically improves the antimicrobial effect of polyquaternium-15 and/or that polyquaternium-15 synergistically improve(s) the antimicrobial effect of 3-hydroxypropyl undecylenate.

According to a preferred embodiment, the mixture comprises or consists of 3-hydroxypropyl undecylenate and methylheptylglycerin, preferably wherein the total amounts of 3-hydroxypropyl undecylenate and methylheptylglycerin, respectively, contained in the mixture are such that 3-hydroxypropyl undecylenate synergistically improves the antimicrobial effect of methylheptylglycerin and/or that methylheptylglycerin synergistically improve(s) the antimicrobial effect of 3-hydroxypropyl undecylenate.

According to a preferred embodiment, the mixture comprises or consists of 3-hydroxypropyl undecylenate and sodium caproyl/lauroyl lactylate, preferably wherein the total amounts of 3-hydroxypropyl undecylenate and sodium caproyl/lauroyl lactylate, respectively, contained in the mixture are such that 3-hydroxypropyl undecylenate synergistically improves the antimicrobial effect of sodium caproyl/lauroyl lactylate and/or that sodium caproyl/lauroyl lactylate synergistically improve(s) the antimicrobial effect of 3-hydroxypropyl undecylenate.

According to a preferred embodiment, the mixture comprises or consists of 3-hydroxypropyl undecylenate and polyglyceryl-10 laurate, preferably wherein the total amounts of 3-hydroxypropyl undecylenate and polyglyceryl-10 laurate, respectively, contained in the mixture are such that 3-hydroxypropyl undecylenate synergistically improves the antimicrobial effect of polyglyceryl-10 laurate and/or that polyglyceryl-10 laurate synergistically improve(s) the antimicrobial effect of 3-hydroxypropyl undecylenate.

According to a preferred embodiment, the mixture comprises or consists of 3-hydroxypropyl undecylenate and glyceryl caprate, preferably wherein the total amounts of 3-hydroxypropyl undecylenate and glyceryl caprate, respectively, contained in the mixture are such that 3-hydroxypropyl undecylenate synergistically improves the antimicrobial effect of glyceryl caprate and/or that glyceryl caprate synergistically improve(s) the antimicrobial effect of 3-hydroxypropyl undecylenate.

According to a preferred embodiment, the mixture comprises or consists of 3-hydroxypropyl undecylenate and polyglyceryl-2 caprate, preferably wherein the total amounts of 3-hydroxypropyl undecylenate and polyglyceryl-2 caprate, respectively, contained in the mixture are such that 3-hydroxypropyl undecylenate synergistically improves the antimicrobial effect of polyglyceryl-2 caprate and/or that polyglyceryl-2 caprate synergistically improve(s) the antimicrobial effect of 3-hydroxypropyl undecylenate.

According to a preferred embodiment, the mixture comprises or consists of 3-hydroxypropyl undecylenate and raspberry ketone, preferably wherein the total amounts of 3-hydroxypropyl undecylenate and raspberry ketone, respectively, contained in the mixture are such that 3-hydroxypropyl undecylenate synergistically improves the antimicrobial effect of raspberry ketone and/or that raspberry ketone synergistically improve(s) the antimicrobial effect of 3-hydroxypropyl undecylenate.

According to a preferred embodiment, the mixture comprises or consists of 3-hydroxypropyl undecylenate and raspberry ketone methyl ether, preferably wherein the total amounts of 3-hydroxypropyl undecylenate and raspberry ketone methyl ether, respectively, contained in the mixture are such that 3-hydroxypropyl undecylenate synergistically improves the antimicrobial effect of and raspberry ketone methyl ether and/or that and raspberry ketone methyl ether synergistically improve(s) the antimicrobial effect of 3-hydroxypropyl undecylenate.

According to a preferred embodiment, the mixture comprises or consists of 3-hydroxypropyl undecylenate and caprylyl caprylate, preferably wherein the total amounts of 3-hydroxypropyl undecylenate and caprylyl caprylate, respectively, contained in the mixture are such that 3-hydroxypropyl undecylenate synergistically improves the antimicrobial effect of caprylyl caprylate and/or that caprylyl caprylate synergistically improve(s) the antimicrobial effect of 3-hydroxypropyl undecylenate.

According to a preferred embodiment, the mixture comprises or consists of 3-hydroxypropyl undecylenate and coco-caprylate/caprate, preferably wherein the total amounts of 3-hydroxypropyl undecylenate and coco-caprylate/caprate, respectively, contained in the mixture are such that 3-hydroxypropyl undecylenate synergistically improves the antimicrobial effect of coco-caprylate/caprate and/or that coco-caprylate/caprate synergistically improve(s) the antimicrobial effect of 3-hydroxypropyl undecylenate.

According to a preferred embodiment, the mixture comprises or consists of 3-hydroxypropyl undecylenate and PEG-8 caprylate, preferably wherein the total amounts of 3-hydroxypropyl undecylenate and PEG-8 caprylate, respectively, contained in the mixture are such that 3-hydroxypropyl undecylenate synergistically improves the antimicrobial effect of PEG-8 caprylate and/or that PEG-8 caprylate synergistically improve(s) the antimicrobial effect of 3-hydroxypropyl undecylenate.

According to a preferred embodiment, the mixture comprises or consists of 3-hydroxypropyl undecylenate and dipropylene glycol caprylate, preferably wherein the total amounts of 3-hydroxypropyl undecylenate and dipropylene glycol caprylate, respectively, contained in the mixture are such that 3-hydroxypropyl undecylenate synergistically improves the antimicrobial effect of dipropylene glycol caprylate and/or that dipropylene glycol caprylate synergistically improve(s) the antimicrobial effect of 3-hydroxypropyl undecylenate.

According to a preferred embodiment, the mixture comprises or consists of 3-hydroxypropyl undecylenate and propylene glycol caprylate, preferably wherein the total amounts of 3-hydroxypropyl undecylenate and propylene glycol caprylate, respectively, contained in the mixture are such that 3-hydroxypropyl undecylenate synergistically improves the antimicrobial effect of propylene glycol caprylate and/or that propylene glycol caprylate synergistically improve(s) the antimicrobial effect of 3-hydroxypropyl undecylenate.

According to a preferred embodiment, the mixture comprises or consists of 3-hydroxypropyl undecylenate and pyridoxine dicaprylate, preferably wherein the total amounts of 3-hydroxypropyl undecylenate and pyridoxine dicaprylate, respectively, contained in the mixture are such that 3-hydroxypropyl undecylenate synergistically improves the antimicrobial effect of pyridoxine dicaprylate and/or that pyridoxine dicaprylate synergistically improve(s) the antimicrobial effect of 3-hydroxypropyl undecylenate.

According to a preferred embodiment, the mixture comprises or consists of 3-hydroxypropyl undecylenate and glyceryl caprylate/caprate, preferably wherein the total amounts of 3-hydroxypropyl undecylenate and glyceryl caprylate/caprate, respectively, contained in the mixture are such that 3-hydroxypropyl undecylenate synergistically improves the antimicrobial effect of glyceryl caprylate/caprate and/or that glyceryl caprylate/caprate synergistically improve(s) the antimicrobial effect of 3-hydroxypropyl undecylenate.

According to a preferred embodiment, the mixture comprises or consists of 3-hydroxypropyl undecylenate and methyl glucose caprylate/caprate, preferably wherein the total amounts of 3-hydroxypropyl undecylenate and methyl glucose caprylate/caprate, respectively, contained in the mixture are such that 3-hydroxypropyl undecylenate synergistically improves the antimicrobial effect of methyl glucose caprylate/caprate and/or that methyl glucose caprylate/caprate synergistically improve(s) the antimicrobial effect of 3-hydroxypropyl undecylenate.

According to a preferred embodiment, the mixture comprises or consists of 3-hydroxypropyl undecylenate and polyglyceryl-3 caprate/caprylate/succinate, preferably wherein the total amounts of 3-hydroxypropyl undecylenate and polyglyceryl-3 caprate/caprylate/succinate, respectively, contained in the mixture are such that 3-hydroxypropyl undecylenate synergistically improves the antimicrobial effect of polyglyceryl-3 caprate/caprylate/succinate and/or that polyglyceryl-3 caprate/caprylate/succinate synergistically improve(s) the antimicrobial effect of 3-hydroxypropyl undecylenate.

According to a preferred embodiment, the mixture comprises or consists of 3-hydroxypropyl undecylenate and polyglyceryl-2 caprylate, preferably wherein the total amounts of 3-hydroxypropyl undecylenate and polyglyceryl-2 caprylate, respectively, contained in the mixture are such that 3-hydroxypropyl undecylenate synergistically improves the antimicrobial effect of polyglyceryl-2 caprylate and/or that polyglyceryl-2 caprylate synergistically improve(s) the antimicrobial effect of 3-hydroxypropyl undecylenate.

According to a preferred embodiment, the mixture comprises or consists of 3-hydroxypropyl undecylenate and polyglyceryl-3 caprylate, preferably wherein the total amounts of 3-hydroxypropyl undecylenate and polyglyceryl-3 caprylate, respectively, contained in the mixture are such that 3-hydroxypropyl undecylenate synergistically improves the antimicrobial effect of polyglyceryl-3 caprylate and/or that polyglyceryl-3 caprylate synergistically improve(s) the antimicrobial effect of 3-hydroxypropyl undecylenate.

According to a preferred embodiment, the mixture comprises or consists of 3-hydroxypropyl undecylenate and polyglyceryl-4 caprylate, preferably wherein the total amounts of 3-hydroxypropyl undecylenate and polyglyceryl-4 caprylate, respectively, contained in the mixture are such that 3-hydroxypropyl undecylenate synergistically improves the antimicrobial effect of polyglyceryl-4 caprylate and/or that polyglyceryl-4 caprylate synergistically improve(s) the antimicrobial effect of 3-hydroxypropyl undecylenate.

According to a preferred embodiment, the mixture comprises or consists of 3-hydroxypropyl undecylenate and polyglyceryl-6 caprylate, preferably wherein the total amounts of 3-hydroxypropyl undecylenate and polyglyceryl-6 caprylate, respectively, contained in the mixture are such that 3-hydroxypropyl undecylenate synergistically improves the antimicrobial effect of polyglyceryl-6 caprylate and/or that polyglyceryl-6 caprylate synergistically improve(s) the antimicrobial effect of 3-hydroxypropyl undecylenate.

According to a preferred embodiment, the mixture comprises or consists of 3-hydroxypropyl undecylenate and polyglyceryl-10 caprylate, preferably wherein the total amounts of 3-hydroxypropyl undecylenate and polyglyceryl-10 caprylate, respectively, contained in the mixture are such that 3-hydroxypropyl undecylenate synergistically improves the antimicrobial effect of polyglyceryl-10 caprylate and/or that polyglyceryl-10 caprylate synergistically improve(s) the antimicrobial effect of 3-hydroxypropyl undecylenate.

According to a preferred embodiment, the mixture comprises or consists of 3-hydroxypropyl undecylenate and polyglyceryl-4 caprylate/caprate, preferably wherein the total amounts of 3-hydroxypropyl undecylenate and polyglyceryl-4 caprylate/caprate, respectively, contained in the mixture are such that 3-hydroxypropyl undecylenate synergistically improves the antimicrobial effect of polyglyceryl-4 caprylate/caprate and/or that polyglyceryl-4 caprylate/caprate synergistically improve(s) the antimicrobial effect of 3-hydroxypropyl undecylenate.

According to a preferred embodiment, the mixture comprises or consists of 3-hydroxypropyl undecylenate and polyglyceryl-6 caprylate/caprate, preferably wherein the total amounts of 3-hydroxypropyl undecylenate and polyglyceryl-6 caprylate/caprate, respectively, contained in the mixture are such that 3-hydroxypropyl undecylenate synergistically improves the antimicrobial effect of polyglyceryl-6 caprylate/caprate and/or that polyglyceryl-6 caprylate/caprate synergistically improve(s) the antimicrobial effect of 3-hydroxypropyl undecylenate.

According to a preferred embodiment, the mixture comprises or consists of 3-hydroxypropyl undecylenate and polyglyceryl-10 caprylate/caprate, preferably wherein the total amounts of 3-hydroxypropyl undecylenate and polyglyceryl-10 caprylate/caprate, respectively, contained in the mixture are such that 3-hydroxypropyl undecylenate synergistically improves the antimicrobial effect of polyglyceryl-10 caprylate/caprate and/or that polyglyceryl-10 caprylate/caprate synergistically improve(s) the antimicrobial effect of 3-hydroxypropyl undecylenate.

According to a preferred embodiment, the mixture comprises or consists of 3-hydroxypropyl undecylenate and propanediol dicaprylate/caprate, preferably wherein the total amounts of 3-hydroxypropyl undecylenate and propanediol dicaprylate/caprate, respectively, contained in the mixture are such that 3-hydroxypropyl undecylenate synergistically improves the antimicrobial effect of propanediol dicaprylate/caprate and/or that propanediol dicaprylate/caprate synergistically improve(s) the antimicrobial effect of 3-hydroxypropyl undecylenate.

According to a preferred embodiment, the mixture comprises or consists of 3-hydroxypropyl undecylenate and xylityl caprate/caprylate, preferably wherein the total amounts of 3-hydroxypropyl undecylenate and xylityl caprate/caprylate, respectively, contained in the mixture are such that 3-hydroxypropyl undecylenate synergistically improves the antimicrobial effect of xylityl caprate/caprylate and/or that xylityl caprate/caprylate synergistically improve(s) the antimicrobial effect of 3-hydroxypropyl undecylenate.

According to a preferred embodiment, the mixture comprises or consists of 3-hydroxypropyl undecylenate and xylityl caprylate, preferably wherein the total amounts of 3-hydroxypropyl undecylenate and xylityl caprylate, respectively, contained in the mixture are such that 3-hydroxypropyl undecylenate synergistically improves the antimicrobial effect of xylityl caprylate and/or that xylityl caprylate synergistically improve(s) the antimicrobial effect of 3-hydroxypropyl undecylenate.

According to a preferred embodiment, the mixture comprises or consists of 3-hydroxypropyl undecylenate and xylityl sesquicaprylate, preferably wherein the total amounts of 3-hydroxypropyl undecylenate and xylityl sesquicaprylate, respectively, contained in the mixture are such that 3-hydroxypropyl undecylenate synergistically improves the antimicrobial effect of xylityl sesquicaprylate and/or that xylityl sesquicaprylate synergistically improve(s) the antimicrobial effect of 3-hydroxypropyl undecylenate.

According to a preferred embodiment, the mixture comprises or consists of 3-hydroxypropyl undecylenate and hydroxyethoxyphenyl butanone, preferably wherein the total amounts of 3-hydroxypropyl undecylenate and hydroxyethoxyphenyl butanone, respectively, contained in the mixture are such that 3-hydroxypropyl undecylenate synergistically improves the antimicrobial effect of hydroxyethoxyphenyl butanone and/or that hydroxyethoxyphenyl butanone synergistically improve(s) the antimicrobial effect of 3-hydroxypropyl undecylenate.

Preferably, the mixture according to the invention contains from 1 to 99.9 wt. %, preferably from 1 to 99 wt. %, more preferably from 1 to 95 wt. %, more preferably from 1 to 90 wt. %, more preferably from 1 to 80 wt. %, more preferably from 1 to 70 wt. %, more preferably from 1 to 60 wt. %, more preferably from 1 to 50 wt. %, most preferably from 1 to 40 wt. %, of constituent (i), based on the total weight of the mixture,
and/or
the mixture contains from 0.1 to 99.9 wt. %, preferably from 0.1 to 90 wt. %, more preferably from 0.1 to 80 wt. %, more preferably from 0.1 to 70 wt. %, more preferably from 0.1 to 60 wt. %, more preferably from 0.1 to 50 wt. %, most preferably from 0.1 to 40 wt. %, of constituent (ii), based on the total weight of the mixture.

Most preferably, the mixture according to the invention contains from 1 to 99.9 wt. %, preferably from 1 to 99 wt. %, more preferably from 1 to 95 wt. %, more preferably from 1 to 90 wt. %, more preferably from 1 to 80 wt. %, more preferably from 1 to 70 wt. %, more preferably from 1 to 60 wt. %, more preferably from 1 to 50 wt. %, most preferably from 1 to 40 wt. %, of constituent (i), based on the total weight of the mixture,
and
the mixture contains from 0.1 to 99.9 wt. %, preferably from 0.1 to 90 wt. %, more preferably from 0.1 to 80 wt. %, more preferably from 0.1 to 70 wt. %, more preferably from 0.1 to 60 wt. %, more preferably from 0.1 to 50 wt. %, most preferably from 0.1 to 40 wt. %, of constituent (ii), based on the total weight of the mixture.

Another aspect of the present invention relates to a semi-finished product comprising or consisting of the mixture according to the invention, i.e. to a semi-finished product comprising or consisting of
(i) one or more compound(s) selected from the group consisting of 3-hydroxypropyl caprylate and 3-hydroxypropyl undecylenate, and
(ii) one or more compound(s) selected from the group consisting of piroctone olamine, climbazole, zinc pyrithione, salicylic acid and salts thereof, decylene glycol, o-cymen-5-ol, selenium sulfide, coal tar, sulphur, ketoconazole, benzoic acid and salts thereof, preferably sodium benzoate, sorbic acid and salts thereof, preferably potassium sorbate, lactic acid and salts thereof, dehydroacetic acid and salts thereof, preferably sodium dehydroacetate, undecylenic acid and salts thereof, levulinic acid and salts thereof, preferably sodium levulinate, anisic acid and salts thereof, phytic acid and salts thereof, preferably sodium phytate, itaconic acid and salts thereof, MEA undecylenate, butyloctanoic acid and salts thereof, citric acid and salts thereof, hydroxyacetophenone, ethylhexylglycerin, caprylhydroxamic acid and salts thereof, undecylenoyl glycine, capryloyl glycine, polyglyceryl-3 caprylate, glyceryl undecylenate, glyceryl caprylate, sorbitan caprylate, glyceryl laurate, phenethyl alcohol, tropolones, 1,2-propanediol, 1,2-butanediol, 1,2-pentanediol (pentylene glycol), 1,2-hexanediol, 1,2-heptanediol, 1,2-octanediol, 1,2-nonanediol, 1,2-decanediol, 1,2-undecanediol, 1,2-dodecanediol, 1,2-tridecanediol, 2,3-pentanediol, 2,3-hexanediol, 2,3-heptanediol, 2,3-octanediol, 2,3-nonanediol, 2,3-decanediol, 2,3-undecanediol, 2,3-dodecanediol, 2,3-tridecanediol, 1,3-propanediol, 1,3-butanediol, 2-methyl-2,4-pentanediol, caprylyl glycol, farnesol, bisabolol, nerolidol, carvacrol, thymol, phenylpropanol, methylbenzyl alcohol, 2-benzylheptanol, 2-methyl 5-cyclohexylpentanol, dimethyl phenylbutanol, PCA ethyl cocoyl arginate, benzyl alcohol, phenoxyethanol, iodopropynyl butylcarbamate, hydroxyethoxyphenyl butanol, ethyl lauroyl arginate HCl, methylchloroisothiazolinone, methylisothiazolinone, methylparaben and salts thereof, preferably sodium methylparaben, ethylparaben and salts thereof, preferably sodium ethylparaben, propylparaben and salts thereof, preferably sodium propylparaben, isopropylparaben and salts thereof, butylparaben and salts thereof, isobutylparaben and salts thereof, DMDM hydantoin, diazolidinyl urea, imidazolidinyl urea, chlorphenesin, disodium EDTA, tetrasodium glutamate diacetate, trisodium ethylenediamine disuccinate, methylglycine N,N-diacetic trisodium salt, glucoheptonic sodium salt, ethanoldiglycinic disodium salt, sodium gluconate, tetrasodium iminodisuccinate, trisodium salt of methylglycine diacetic acid, polyaspartic acid sodium salt, phenoxyisopropanol, chlorhexidine, chlorhexidine digluconate, triclocarban, triclosan, O-phenylphenol, 4-hydroxybenzoic acid, TEA-salicylate, benzyl salicylate, hexamidine, hexamidine diisethionate, dichlorobenzyl alcohol, ethyl benzoate, sodium paraben, isothiazolinones, p-chloro-m-cresol, cetrimonium chloride, cetrimonium bromide, benzethonium chloride, alkyltrimonium bromide, alkyltrimonium chloride, benzalkonium chloride, benzalkonium bromide, myristamidopropyl PG-dimonium chloride phosphate, guar hydroxypropyltrimonium chloride, hydroxypropyl guar hydroxypropyltrimonium chloride, polyquaternium-6, polyquaternium-7, polyquaternium-10, polyquaternium-11, polyquaternium-12, polyquaternium-15, methylheptylglycerin, sodium caproyl/lauroyl lactylate, polyglyceryl-10 laurate, glyceryl caprate, polyglyceryl-2 caprate, raspberry ketone, raspberry ketone methyl ether, caprylyl caprylate, coco-caprylate/caprate, PEG-8 caprylate, dipropylene glycol caprylate, propylene glycol caprylate, pyridoxine dicaprylate, glyceryl caprylate/caprate, methyl glucose caprylate/caprate, polyglyceryl-3 caprate/caprylate/succinate, polyglyceryl-2 caprylate, polyglyceryl-3 caprylate, polyglyceryl-4 caprylate, polyglyceryl-6 caprylate, polyglyceryl-10 caprylate, polyglyceryl-4 caprylate/caprate, polyglyceryl-6 caprylate/caprate, polyglyceryl-10 caprylate/caprate, propanediol dicaprylate/caprate, xylityl caprate/caprylate, xylityl caprylate, xylityl sesquicaprylate, and hydroxyethoxyphenyl butanone,
preferably wherein the total amounts of constituents (i) and (ii), respectively, contained in the semi-finished product are such that constituent (i) synergistically improves the antimicrobial effect of constituent (ii) and/or that constituent (ii) synergistically improve(s) the antimicrobial effect of constituent (i).

Another aspect of the present invention relates to a product, preferably a solid or liquid personal care product as defined herein, comprising or consisting of the mixture according to the invention, i.e. to a solid or liquid personal care product comprising or consisting of
(i) one or more compound(s) selected from the group consisting of 3-hydroxypropyl caprylate and 3-hydroxypropyl undecylenate, and
(ii) one or more compound(s) selected from the group consisting of piroctone olamine, climbazole, zinc pyrithione, salicylic acid and salts thereof, decylene glycol, o-cymen-5-ol, selenium sulfide, coal tar, sulphur, ketoconazole, benzoic acid and salts thereof, preferably sodium benzoate, sorbic acid and salts thereof, preferably potassium sorbate, lactic acid and salts thereof, dehydroacetic acid and salts thereof, preferably sodium dehydroacetate, undecylenic acid and salts thereof, levulinic acid and salts thereof, preferably sodium levulinate, anisic acid and salts thereof, phytic acid and salts thereof, preferably sodium phytate, itaconic acid and salts thereof, MEA undecylenate, butyloctanoic acid and salts thereof, citric acid and salts thereof, hydroxyacetophenone, ethylhexylglycerin, caprylhydroxamic acid and salts thereof, undecylenoyl glycine, capryloyl glycine, polyglyceryl-3 caprylate, glyceryl undecylenate, glyceryl caprylate, sorbitan caprylate, glyceryl laurate, phenethyl alcohol, tropolones, 1,2-propanediol, 1,2-butanediol, 1,2-pentanediol (pentylene glycol), 1,2-hexanediol, 1,2-heptanediol, 1,2-octanediol, 1,2-nonanediol, 1,2-decanediol, 1,2-undecanediol, 1,2-dodecanediol, 1,2-tridecanediol, 2,3-pentanediol, 2,3-hexanediol, 2,3-heptanediol, 2,3-octanediol, 2,3-nonanediol, 2,3-decanediol, 2,3-undecanediol, 2,3-dodecanediol, 2,3-tridecanediol, 1,3-propanediol, 1,3-butanediol, 2-methyl-2,4-pentanediol, caprylyl glycol, farnesol, bisabolol, nerolidol, carvacrol, thymol, phenylpropanol, methylbenzyl alcohol, 2-benzylheptanol, 2-methyl 5-cyclohexylpentanol, dimethyl phenylbutanol, PCA ethyl cocoyl arginate, benzyl alcohol, phenoxyethanol, iodopropynyl butylcarbamate, hydroxyethoxyphenyl butanol, ethyl lauroyl arginate HCl, methylchloroisothiazolinone, methylisothiazolinone, methylparaben and salts thereof, preferably sodium methylparaben, ethylparaben and salts thereof, preferably sodium ethylparaben, propylparaben and salts thereof, preferably sodium propylparaben, isopropylparaben and salts thereof, butylparaben and salts thereof, isobutylparaben and salts thereof, DMDM hydantoin, diazolidinyl urea, imidazolidinyl urea, chlorphenesin, disodium EDTA, tetrasodium glutamate diacetate, trisodium ethylenediamine disuccinate, methylglycine N,N-diacetic trisodium salt, glucoheptonic sodium salt, ethanoldiglycinic disodium salt, sodium gluconate, tetrasodium iminodisuccinate, trisodium salt of methylglycine diacetic acid, polyaspartic acid sodium salt, phenoxyisopropanol, chlorhexidine, chlorhexidine digluconate, triclocarban, triclosan, O-phenylphenol, 4-hydroxybenzoic acid, TEA-salicylate, benzyl salicylate, hexamidine, hexamidine diisethionate, dichlorobenzyl alcohol, ethyl benzoate, sodium paraben, isothiazolinones, p-chloro-m-cresol, cetrimonium chloride, cetrimonium bromide, benzethonium chloride, alkyltrimonium bromide, alkyltrimonium chloride, benzalkonium chloride, benzalkonium bromide, myristamidopropyl PG-dimonium chloride phosphate, guar hydroxypropyltrimonium chloride, hydroxypropyl guar hydroxypropyltrimonium chloride, polyquaternium-6, polyquaternium-7, polyquaternium-10, polyquaternium-11, polyquaternium-12, polyquaternium-15, methylheptylglycerin, sodium caproyl/lauroyl lactylate, polyglyceryl-10 laurate, glyceryl caprate, polyglyceryl-2 caprate, raspberry ketone, raspberry ketone methyl ether, caprylyl caprylate, coco-caprylate/caprate, PEG-8 caprylate, dipropylene glycol caprylate, propylene glycol caprylate, pyridoxine dicaprylate, glyceryl caprylate/caprate, methyl glucose caprylate/caprate, polyglyceryl-3 caprate/caprylate/succinate, polyglyceryl-2 caprylate, polyglyceryl-3 caprylate, polyglyceryl-4 caprylate, polyglyceryl-6 caprylate, polyglyceryl-10 caprylate, polyglyceryl-4 caprylate/caprate, polyglyceryl-6 caprylate/caprate, polyglyceryl-10 caprylate/caprate, propanediol dicaprylate/caprate, xylityl caprate/caprylate, xylityl caprylate, xylityl sesquicaprylate, and hydroxyethoxyphenyl butanone,
preferably wherein the total amounts of constituents (i) and (ii), respectively, contained in the product are such that constituent (i) synergistically improves the antimicrobial effect of constituent (ii) and/or that constituent (ii) synergistically improve(s) the antimicrobial effect of constituent (i).

Preferably, the personal care product according to the invention contains from 0.01 to 10 wt. %, preferably from 0.02 to 5 wt. %, more preferably from 0.05 to 3 wt. %, more preferably from 0.1 to 2 wt. %, of constituent (i), based on the total weight of the product,
and/or
the personal care product contains from 0.001 to 20 wt. %, preferably from 0.005 to 15 wt. %, more preferably from 0.009 to 10 wt. %, most preferably from 0.01 to 5 wt. %, of constituent (ii), based on the total weight of the product.

Most preferably, the personal care product according to the invention contains from 0.01 to 10 wt. %, preferably from 0.02 to 5 wt. %, more preferably from 0.05 to 3 wt. %, more preferably from 0.1 to 2 wt. %, of constituent (i), based on the total weight of the product,
and
the personal care product contains from 0.001 to 20 wt. %, preferably from 0.005 to 15 wt. %, more preferably from 0.009 to 10 wt. %, most preferably from 0.01 to 5 wt. %, of constituent (ii), based on the total weight of the product.

Preferred embodiments of the uses according to the first aspect of the invention correspond to or can be derived from the preferred embodiments of the uses according to the second aspect of the invention, which are explained above, or vice versa.

Preferred embodiments of the processes according to the third aspect of the invention correspond to or can be derived from the preferred embodiments of the processes according to the fourth or fifth aspect of the invention, which are explained above, or vice versa.

Preferred embodiments of the mixtures according to the invention correspond to or can be derived from the preferred embodiments of the semi-finished products according to the invention, which are explained above, or vice versa.

Preferred embodiments of the mixtures according to the invention correspond to or can be derived from the preferred embodiments of the products according to the invention, which are explained above, or vice versa.

Preferred embodiments of the uses according to the invention correspond to or can be derived from the preferred embodiments of the processes, mixtures, semi-finished products and products according to the invention, which are explained above, or vice versa.

The invention will now be described in more detail hereinafter with reference to the examples. Further aspects of the present invention are disclosed in the accompanying claims.

### Examples

### Methods

Preservative efficacy tests were performed according to the European Pharmacopoeia with adjusted inoculation numbers (10⁶-10⁷ cells of suitable bacteria or fungi). Material was tested in predesigned standardised cosmetic formulations either as oil-in-water anionic emulsion (Table 2), shampoo (Table 3), or wet wipe solution (Table 4). The antimicrobial actives were dissolved or mixed with the standardised cosmetic formulations, leading to the indicated test concentrations (in w/w %, cf. Tables 5 to 12 below). To determine synergistic indices, raw values for bacterial of fungal colony counts were used.

TABLE 2
O/W anionic emulsion

| Ingredient | w/w % |
|---|---|
| Carbomer | 0.2 |
| Stearyl Heptanoate | 4.0 |
| Ethylhexyl Isononanoate | 5.0 |
| Stearyl Alcohol | 2.0 |
| Glyceryl Stearate Citrate | 3.0 |
| Sodium Hydroxyide, 10% | 0.3 |
| aqueous sol. | |
| Water (Aqua) | ad 100 |

TABLE 3
Shampoo

| Ingredient | w/w % |
|---|---|
| Sodium Laureth Sulfate | 17 |
| Citric Acid, 10% aqueous sol. | 0.15 |
| Sodium Chloride | 0.4 |
| Cocoamidopropyl Betaine | 5 |
| Water (Aqua) | Ad 100 |

TABLE 4
Wet wipe solution

| Ingredient | w/w % |
|---|---|
| Propylenglycol | 8 |
| Solubiliser | 2 |
| PEG-40 Hydrogenated Castor Oil, | |
| Trideceth-9, Propylene Glycol, | |
| Water (Aqua) | |
| Citric Acid, 10% sol. | 0.1 |
| Water (Aqua) | ad 100 |

To calculate the synergism between the indicated antimicrobial actives (cf. preservative efficacy tests below), the Kull equation (Kull et al., Applied Microbiology 1961, 9 (6), pp. 538-541, DOI: 10.1128/am.9.6.538-541.1961.; Steinberg D. C. (2000): Measuring Synergy. In Cosmetics & Toiletries, 115) was used:

$$SI = ( {C}_{mixture} \times {P}_{A} ) / {C}_{A} ) + ( {C}_{mixture} \times {P}_{B} ) / {C}_{B}$$

where
SI is the synergism index according to Kull
C_{A} is the cell count for substance A (also “count A”)
C_{B} is the cell count for substance B (also “count B”)
Cₘᵢₓₜᵤᵣₑ is the cell count for the mixture of substance A and B (also “count mix”)
P_{A} is the proportion of the substance A in the mixture in relation to the concentration tested alone
P_{B} is the proportion of the substance B in the mixture in relation to the concentration tested alone

TABLE 5
Preservative efficacy testing of 3-hydroxypropyl caprylate, piroctone olamine and a
mixture of 3-hydroxypropyl caprylate and piroctone olamine in an O/W anionic emulsion

| | | | | | Absolute cell counts | | | | Relative cell counts | | | |
|---|---|---|---|---|---|---|---|---|---|---|---|---|
| Formulation: O/W emulsion, anionic | | | | | 2 | 7 | 14 | 28 | 2 | 7 | 14 | 28 |
| Substance | P_{A} | P_{B} | Organism | Inoculum | days | days | days | days | days | days | days | days |
| 0.5% | X | | S. aureus | 5.50E+06 | 2.10E+05 | 1.80E+02 | 1 | 1 | 0.0382 | 0.0000 | 0.0000 | 0.0000 |
| 3-Hydroxypropyl | | | E. coli | 3.40E+06 | 3.30E+06 | 2.60E+04 | 1 | 1 | 0.9706 | 0.0076 | 0.0000 | 0.0000 |
| caprylate | | | | | | | | | | | | |
| 0.5% | | | S. aureus | 3.20E+06 | 2.50E+06 | 3.80E+04 | 1 | 1 | 0.7813 | 0.0119 | 0.0000 | 0.0000 |
| Piroctone olamine | | | E. coli | 3.10E+06 | 1 | 1 | 1 | 1 | 0.0000 | 0.0000 | 0.0000 | 0.0000 |
| 0.25% | 0.5 | 0.5 | S. aureus | 3.30E+06 | 1.20E+05 | 1 | 1 | 1 | 0.0364 | 0.0000 | 0.0000 | 0.0000 |
| 3-Hydroxypropyl | 0.5 | 0.5 | E. coli | 3.80E+06 | 1 | 1 | 1 | 1 | 0.0000 | 0.0000 | 0.0000 | 0.0000 |
| caprylate | | | | | | | | | | | | |
| 0.25% | | | | | | | | | | | | |
| Piroctone olamine | | | | | | | | | | | | |

| SI = (Count Mix × P_{A})/count A + (count mix × P_{B})/count B | S. aureus | 0.4995 | 0.0046 | nd | nd | Synergy |
|---|---|---|---|---|---|---|
| | E. coli | 0.4079 | 0.4079 | nd | nd | Synergy |

TABLE 6
Preservative efficacy testing of 3-hydroxypropyl caprylate, piroctone olamine and a
mixture of 3-hydroxypropyl caprylate and piroctone olamine in a shampoo formulation

| | | | | | Absolute cell counts | | | | Relative cell counts | | | |
|---|---|---|---|---|---|---|---|---|---|---|---|---|
| Formulation: Shampoo | | | | | 2 | 7 | 14 | 28 | 2 | 7 | 14 | 28 |
| Substance | P_{A} | P_{B} | Organism | Inoculum | days | days | days | days | days | days | days | days |
| 0.5% | X | | P. aeruginosa | 4.40E+06 | 9.30E+04 | 9.50E+02 | 1 | 1 | 0.0211 | 0.0002 | 0.0000 | 0.0000 |
| 3-Hydroxypropyl | | | C. albicans | 2.20E+06 | 2.10E+03 | 1 | 1 | 1 | 0.0010 | 0.0000 | 0.0000 | 0.0000 |
| caprylate | | | | | | | | | | | | |
| 0.5% | | | P. aeruginosa | 4.40E+06 | 1 | 1 | 1 | 1 | 0.0000 | 0.0000 | 0.0000 | 0.0000 |
| Piroctone olamine | | | C. albicans | 2.20E+06 | 1 | 1 | 1 | 1 | 0.0000 | 0.0000 | 0.0000 | 0.0000 |
| 0.25% | 0.5 | 0.5 | P. aeruginosa | 3.30E+06 | 1 | 1 | 1 | 1 | 0.0000 | 0.0000 | 0.0000 | 0.0000 |
| 3-Hydroxypropyl | 0.5 | 0.5 | C. albicans | 2.00E+06 | 1 | 1 | 1 | 1 | 0.0000 | 0.0000 | 0.0000 | 0.0000 |
| caprylate | | | | | | | | | | | | |
| 0.25% | | | | | | | | | | | | |
| Piroctone olamine | | | | | | | | | | | | |

| SI = (Count Mix × P_{A})/count A + (count mix × P_{B})/count B | P. aeruginosa | 0.6667 | 0.6674 | nd | nd | Synergy |
|---|---|---|---|---|---|---|
| | C. albicans | 0.5503 | nd | nd | nd | Synergy |

TABLE 7
Preservative efficacy testing of 3-hydroxypropyl caprylate, salicylic acid and a
mixture of 3-hydroxypropyl caprylate and salicylic acid in a shampoo formulation

| | | | | | Absolute cell counts | | | | Relative cell counts | | | |
|---|---|---|---|---|---|---|---|---|---|---|---|---|
| Formulation: Shampoo | | | | | 2 | 7 | 14 | 28 | 2 | 7 | 14 | 28 |
| Substance | P_{A} | P_{B} | Organism | Inoculum | days | days | days | days | days | days | days | days |
| 0.5% 3- | X | | P. aeruginosa | 4.40E+06 | 9.30E+04 | 9.50E+02 | 1 | 1 | 0.0211 | 0.0002 | 0.0000 | 0.0000 |
| Hydroxypropyl | | | E. coli | 3.10E+06 | 1.80E+06 | 2.00E+06 | 1.70E+06 | 1.10E+06 | 0.5806 | 0.6452 | 0.5484 | 0.3548 |
| caprylate | | | C. albicans | 2.20E+06 | 2.10E+03 | 1 | 1 | 1 | 0.0010 | 0.0000 | 0.0000 | 0.0000 |
| 0.5% Salicylic | | | P. aeruginosa | 3.70E+06 | 1 | 1 | 1 | 1 | 0.0000 | 0.0000 | 0.0000 | 0.0000 |
| Acid | | | E. coli | 4.50E+06 | 9.50E+03 | 7.20E+03 | 1 | 1 | 0.0021 | 0.0016 | 0.0000 | 0.0000 |
| | | | C. albicans | 2.10E+06 | 7.30E+01 | 1 | 1 | 1 | 0.0000 | 0.0000 | 0.0000 | 0.0000 |
| 0.25% 3- | 0.5 | 0.5 | P. aeruginosa | 3.70E+06 | 1 | 1 | 1 | 1 | 0.0000 | 0.0000 | 0.0000 | 0.0000 |
| Hydroxypropyl | 0.5 | 0.5 | E. coli | 4.50E+06 | 1 | 1 | 1 | 1 | 0.0000 | 0.0000 | 0.0000 | 0.0000 |
| caprylate | 0.5 | 0.5 | C. albicans | 2.10E+06 | 1 | 1 | 1 | 1 | 0.0000 | 0.0000 | 0.0000 | 0.0000 |
| 0.25% | | | | | | | | | | | | |
| Salicylic Acid | | | | | | | | | | | | |

| SI = (Count Mix × P_{A})/ | P. aeruginosa | 0.5000 | 0.5006 | nd | nd | Synergy |
|---|---|---|---|---|---|---|
| count A + (count mix × P_{B})/count B | E. coli | 0.0001 | 0.0001 | 0.5000 | 0.5000 | Synergy |
| | C. albicans | 0.0071 | nd | nd | nd | Synergy |

TABLE 8
Preservative efficacy testing of 3-hydroxypropyl caprylate, sodium benzoate and a
mixture of 3-hydroxypropyl caprylate and sodium benzoate in a shampoo formulation

| | | | | | Absolute cell counts | | | | Relative cell counts | | | |
|---|---|---|---|---|---|---|---|---|---|---|---|---|
| Formulation: Shampoo | | | | | 2 | 7 | 14 | 28 | 2 | 7 | 14 | 28 |
| Substance | P_{A} | P_{B} | Organism | Inoculum | days | days | days | days | days | days | days | days |
| 0.5% 3- | X | | P. aeruginosa | 4.40E+06 | 9.30E+04 | 9.50E+02 | 1 | 1 | 0.0211 | 0.0002 | 0.0000 | 0.0000 |
| Hydroxypropyl | | | E. coli | 3.10E+06 | 1.80E+06 | 2.00E+06 | 1.70E+06 | 1.10E+06 | 0.5806 | 0.6452 | 0.5484 | 0.3548 |
| caprylate | | | | | | | | | | | | |
| 0.5% Sodium | | | P. aeruginosa | 3.70E+06 | 9.60E+03 | 1 | 1 | 1 | 0.0026 | 0.0000 | 0.0000 | 0.0000 |
| Benzoate | | | E. coli | 4.50E+06 | 8.70E+05 | 9.50E+05 | 4.20E+05 | 2.40E+04 | 0.1933 | 0.2111 | 0.0933 | 0.0053 |
| 0.25% 3- | 0.5 | 0.5 | P. aeruginosa | 3.70E+06 | 3.00E+03 | 1 | 1 | 1 | 0.0008 | 0.0000 | 0.0000 | 0.0000 |
| Hydroxypropyl | 0.5 | 0.5 | E. coli | 4.50E+06 | 4.50E+05 | 7.00E+05 | 4.80E+05 | 4.10E+03 | 0.1000 | 0.1556 | 0.1067 | 0.0009 |
| caprylate | | | | | | | | | | | | |
| 0.25% Sodium | | | | | | | | | | | | |
| Benzoate | | | | | | | | | | | | |

| | | | | | | |
|---|---|---|---|---|---|---|
| SI = (Count Mix × P_{A})/ | P. aeruginosa | 0.1754 | 0.5006 | nd | nd | Synergy |
| count A + (count mix × P_{B})/count B | E. coli | 0.3447 | 0.4890 | 0.6687 | 0.0867 | Synergy |

TABLE 9
Preservative efficacy testing of 3-hydroxypropyl caprylate, sodium benzoate and a
mixture of 3-hydroxypropyl caprylate and sodium benzoate in a shampoo formulation

| | | | | | Absolute cell counts | | | | Relative cell counts | | | |
|---|---|---|---|---|---|---|---|---|---|---|---|---|
| Formulation: Shampoo | | | | | 2 | 7 | 14 | 28 | 2 | 7 | 14 | 28 |
| Substance | P_{A} | P_{B} | Organism | Inoculum | days | days | days | days | days | days | days | days |
| 0.5% 3- | X | | S. aureus | 3.20E+06 | 1 | 1 | 1 | 1 | 0.0000 | 0.0000 | 0.0000 | 0.0000 |
| Hydroxypropyl | | | A. | 3.30E+06 | 3.00E+06 | 3.80E+06 | 4.50E+06 | 8.10E+05 | 0.9091 | 1.1515 | 1.3636 | 0.2455 |
| caprylate | | | brasiliensis | | | | | | | | | |
| 0.2% Sodium | | | S. aureus | 5.90E+06 | 2.20E+04 | 1 | 1 | 1 | 0.0037 | 0.0000 | 0.0000 | 0.0000 |
| Benzoate | | | A. | 1.00E+06 | 8.00E+04 | 1.10E+04 | 3.30E+02 | 3.20E+02 | 0.0800 | 0.0110 | 0.0003 | 0.0003 |
| | | | brasiliensis | | | | | | | | | |
| 0.5% 3- | 1 | ′2.5 | S. aureus | 4.10E+06 | 1 | 1 | 1 | 1 | 0.0000 | 0.0000 | 0.0000 | 0.0000 |
| Hydroxypropyl | 1 | ′2.5 | A. | 4.10E+06 | 5.10E+03 | 1 | 1 | 1 | 0.0012 | 0.0000 | 0.0000 | 0.0000 |
| caprylate | | | brasiliensis | | | | | | | | | |
| 0.5% Sodium | | | | | | | | | | | | |
| Benzoate | | | | | | | | | | | | |

| | | | | | | |
|---|---|---|---|---|---|---|
| SI = (Count Mix × P_{A})/ | S. aureus | 0.7807 | nd | nd | nd | Synergy |
| count A + (count mix × P_{B})/count B | A. brasiliensis | 0.0402 | 0.0001 | 0.0018 | 0.0019 | Synergy |

TABLE 10
Preservative efficacy testing of 3-hydroxypropyl caprylate, potassium sorbate and a
mixture of 3-hydroxypropyl caprylate and potassium sorbate in a shampoo formulation

| | | | | | Absolute cell counts | | | | Relative cell counts | | | |
|---|---|---|---|---|---|---|---|---|---|---|---|---|
| Formulation: Shampoo | | | | | 2 | 7 | 14 | 28 | 2 | 7 | 14 | 28 |
| Substance | P_{A} | P_{B} | Organism | Inoculum | days | days | days | days | days | days | days | days |
| 0.5% 3- | X | | A. brasiliensis | 3.30E+06 | 3.00E+06 | 3.80E+06 | 4.50E+06 | 8.10E+05 | 0.9091 | 1.1515 | 1.3636 | 0.2455 |
| Hydroxypropyl | | | | | | | | | | | | |
| caprylate | | | | | | | | | | | | |
| 0.5% | | | A. brasiliensis | 4.10E+06 | 1.40E+04 | 1 | 1 | 1 | 0.0034 | 0.0000 | 0.0000 | 0.0000 |
| Potassium | | | | | | | | | | | | |
| Sorbate | | | | | | | | | | | | |
| 0.5% 3- | 1 | 1 | A. brasiliensis | 4.10E+06 | 5.10E+03 | 1 | 1 | 1 | 0.0012 | 0.0000 | 0.0000 | 0.0000 |
| Hydroxypropyl | | | | | | | | | | | | |
| caprylate | | | | | | | | | | | | |
| 0.5% | | | | | | | | | | | | |
| Potassium | | | | | | | | | | | | |
| Sorbate | | | | | | | | | | | | |

| | | | | | | |
|---|---|---|---|---|---|---|
| SI = (Count Mix × P_{A})/ | A. brasiliensis | 0.3657 | 1.0000 | 1.0000 | 1.0000 | Synergy |
| count A + (count mix × P_{B})/count B | | | | | | |

TABLE 11
Preservative efficacy testing of 3-hydroxypropyl caprylate, decylene glycol and a
mixture of 3-hydroxypropyl caprylate and decylene glycol in a shampoo formulation

| | | | | | Absolute cell counts | | | | Relative cell counts | | | |
|---|---|---|---|---|---|---|---|---|---|---|---|---|
| Formulation: Shampoo | | | | | 2 | 7 | 14 | 28 | 2 | 7 | 14 | 28 |
| Substance | P_{A} | P_{B} | Organism | Inoculum | days | days | days | days | days | days | days | days |
| 0.5% 3- | X | | A. brasiliensis | 3.30E+06 | 3.00E+06 | 3.80E+06 | 4.50E+06 | 8.10E+05 | 0.9091 | 1.1515 | 1.3636 | 0.2455 |
| Hydroxypropyl | | | | | | | | | | | | |
| caprylate | | | | | | | | | | | | |
| 0.5% | | | A. brasiliensis | 4.10E+06 | 7.90E+04 | 8.60E+04 | 7.50E+04 | 6.20E+04 | 0.0193 | 0.0210 | 0.0183 | 0.0151 |
| Decylene | | | | | | | | | | | | |
| Glycol | | | | | | | | | | | | |
| 0.25% 3- | 0.5 | 0.5 | A. brasiliensis | 4.10E+06 | 1.20E+05 | 4.80E+04 | 3.90E+04 | 3.40E+04 | 0.0293 | 0.0117 | 0.0095 | 0.0083 |
| Hydroxypropyl | | | | | | | | | | | | |
| caprylate | | | | | | | | | | | | |
| 0.25% | | | | | | | | | | | | |
| Decylene | | | | | | | | | | | | |
| Glycol | | | | | | | | | | | | |

| | | | | | | |
|---|---|---|---|---|---|---|
| SI = (Count Mix × P_{A})/ | A. brasiliensis | 0.7756 | 0.2842 | 0.2635 | 0.2911 | Synergy |
| count A + (count mix × P_{B})/count B | | | | | | |

TABLE 12
Preservative efficacy testing of 3-hydroxypropyl caprylate, climbazole and a
mixture of 3-hydroxypropyl caprylate and climbazole in a shampoo formulation

| | | | | | Absolute cell counts | | | | Relative cell counts | | | |
|---|---|---|---|---|---|---|---|---|---|---|---|---|
| Formulation: Shampoo | | | | | 2 | 7 | 14 | 28 | 2 | 7 | 14 | 28 |
| Substance | P_{A} | P_{B} | Organism | Inoculum | days | days | days | days | days | days | days | days |
| 0.5% 3- | X | | A. | 3.30E+06 | 3.00E+06 | 3.80E+06 | 4.50E+06 | 8.10E+05 | 0.9091 | 1.1515 | 1.3636 | 0.2455 |
| Hydroxypropyl | | | brasiliensis | | | | | | | | | |
| caprylate | | | | | | | | | | | | |
| 0.5% | | | A. | 4.10E+06 | 1.50E+05 | 6.20E+04 | 5.90E+04 | 4.60E+04 | 0.0366 | 0.0151 | 0.0144 | 0.0112 |
| Climbazole | | | brasiliensis | | | | | | | | | |
| 0.25% 3- | 0.5 | 0.5 | A. | 4.10E+06 | 9.50E+04 | 2.90E+04 | 3.50E+04 | 3.10E+04 | 0.0232 | 0.0071 | 0.0085 | 0.0076 |
| Hydroxypropyl | | | brasiliensis | | | | | | | | | |
| caprylate 0.25% | | | | | | | | | | | | |
| Climbazole | | | | | | | | | | | | |

| | | | | | | |
|---|---|---|---|---|---|---|
| SI = (Count Mix × P_{A})/ | A. brasiliensis | 0.3294 | 0.2369 | 0.2997 | 0.3524 | Synergy |
| count A + (count mix × P_{B})/count B | | | | | | |

### Formulation Examples

1=Well ageing Face Cream, soft touch
2=Daily emulsion with SPF 30, UVA/UVB balanced
3=Protecting and scalp soothing hair conditioner, rinse-off
4=Barrier strengthening oil in cream shower gel
5=Anti dandruff shampoo
6=Well-ageing eye contour serum
7=Natural beauty enhancer (solid concentrate)
8=Refreshing gel with UV protection
9=Hand and body cream
10=Natural beauty protecting soft wet wipes
11=Weather protection cream
12=Men after shave fluid
13=Reinforcing purifying mask, rinse-off
14=Cool relief deo
15=Skin well-being body lotion
16=Refreshing eau micellaire
17=Reinforcing sun protection mist exp. SPF30, UVA/UVB balance
18=Sport fit deo
19=Beach time lotion SFP 30
20=Antiperspirant

In formulations 1 to 20, the following perfume oils (fragrances) PO1 and PO2 were used. Each formulation was prepared with PO1 and with PO2 separately, wherein the same total amounts of PO1 or, respectively, PO2 were used.

TABLE 13
Composition of perfume oil 1 (PO1, amounts in ‰ b.w.)

| Ingredients | Amount |
|---|---|
| ALDEHYDE C14 SO-CALLED | 2 |
| ALLYL AMYL GLYCOLATE 10% DPG | 5 |
| ANISIC ALDEHYDE PURE | 5 |
| APPLE OLIFFAC TYPE | 10 |
| BENZYLACETATE | 50 |
| BERGAMOT IDENTOIL ® COLOURLESS | 15 |
| CANTHOXAL | 5 |
| CETALOX 10% IPM | 3 |
| CITRONELLOL 950 | 40 |
| DAMASCENONE TOTAL 1% DPG | 5 |
| DAMASCONE ALPHA 10% DPG | 5 |
| DAMASCONE DELTA 10% DPG | 2 |
| DIMETHYL BENZYL CARBINYL BUTYRATE | 2 |
| DIPROPYLENE GLYCOL | 178 |
| EBANOL | 2 |
| ETHYL DECADIENOATE TRANS CIS-2,4 10% IPM | 2 |
| FLOROSA | 5 |
| FRAMBINON ® 10% DPG (Raspberry Ketone) | 7 |
| GALAXOLIDE 50% IN IPM | 100 |
| GALBEX TYPE BASE | 1 |
| GERANYL ACETATE PURE | 2 |
| HEDIONE | 30 |
| HELIOTROPIN | 10 |
| HEXENYL ACETATE CIS-3 10% DPG | 1 |
| HEXENYL SALICYLATE CIS-3 | 5 |
| HEXYL CINNAMIC ALDEHYDE ALPHA | 70 |
| HEXYL SALICYLATE | 50 |
| HYDROXY CITRONELLAL | 10 |
| ISO E SUPER | 15 |
| ISORALDEINE 70 | 20 |
| LEAFOVERT ® | 1 |
| LILIAL | 60 |
| LINALOOL | 60 |
| LINALYL ACETATE | 20 |
| LYRAL | 7 |
| MANZANATE | 2 |
| PHENOXANOL | 7 |
| PHENYLETHYL ALCOHOL | 120 |
| SANDAL MYSORE CORE | 2 |
| SANDRANOL ® | 7 |
| STYRALYL ACETATE | 3 |
| TAGETES RCO 10% TEC | 2 |
| TERPINEOL PURE | 20 |
| TETRAHYDROGERANIOL 10% DPG | 5 |
| TONALIDE | 7 |
| VERTOCITRAL 10% DPG | 5 |
| VERTOFIX | 15 |
| Total | 1000 |

TABLE 14
Composition of perfume oil 2 (PO2, amounts in ‰ b.w.)

| Ingredients | Amount |
|---|---|
| AMBRETTOLIDE (MACRO) | 10 |
| AMBROXIDE 10% in IPM | 10 |
| BENZYL ACETATE | 20 |
| BENZYL SALICYLATE | 15 |
| BERGAMOT OIL. Bergapten-free | 60 |
| CALONE ® 1951 10% in DPG | 15 |
| COUMARIN | 5 |
| CYCLOGALBANATE ® 10% in DPG | 10 |
| ALPHA -DAMASCONE 1% in DPG | 20 |
| DIHYDROMYRCENOL | 10 |
| ETHYL LINALOOL | 75 |
| ETHYL LINALYLACETATE | 50 |
| ETHYL MALTOL 1% in DEP | 10 |
| ETHYLENE BRASSYLATE (MACRO) | 80 |
| FLOROSA | 40 |
| GERANYLACETATE | 10 |
| HEDIONE ® HC/30 | 35 |
| HEDIONE ® | 210 |
| HELIONAL ® | 15 |
| HELVETOLIDE ® (ALICYC) | 30 |
| HEXENYLSALICYLATE CIS-3 | 20 |
| ISO E SUPER ® | 40 |
| LEAFOVERT ® 10% in DEP | 10 |
| LILIAL ® | 80 |
| LYRAL ® | 20 |
| MANDARIN OIL | 10 |
| STYRALYL ACETATE | 5 |
| SYMROSE ® | 15 |
| VANILLIN 10% in DEP | 20 |
| DIPROPYLENE GLYCOL (DPG) | 50 |
| TOTAL | 1000 |

TABLE 15
Cosmetic formulations 1 to 11 (amounts in % b.w.)

| Ingredients | Amounts [wt.-%] | | | | | | | | | | |
|---|---|---|---|---|---|---|---|---|---|---|---|
| INCI name | 1 | 2 | 3 | 4 | 5 | 6 | 7 | 8 | 9 | 10 | 11 |
| Acusol OP 301 | | | | 1.0 | | | | | | | |
| Styrene/Acrylates | | | | | | | | | | | |
| Copolymer | | | | | | | | | | | |
| Aloe Vera Gel | | | | | | | | 0.3 | | | |
| Concentrate 10/1 | | | | | | | | | | | |
| Aloe Barbadensis Leaf | | | | | | | | | | | |
| Juice | | | | | | | | | | | |
| Amigum | | | | | | 0.2 | | | | | |
| Sclerotium Gum | | | | | | | | | | | |
| Antil 120 Plus | | | | 1.0 | | | | | | | |
| PEG-120 Methyl Glucose | | | | | | | | | | | |
| Dioleate | | | | | | | | | | | |
| Apricot Kernel Oil | | | 0.5 | | | | | | | | |
| Prunus Armeniaca Kernel | | | | | | | | | | | |
| Oil | | | | | | | | | | | |
| Argan Oil | | | 0.5 | | | | | | | | |
| Argania Spinosa Kernel Oil | | | | | | | | | | | |
| Aristoflex ® AVC | | | | | | | | 1.4 | | | |
| Ammonium | | | | | | | | | | | |
| acryloyoldimethyltaurate/vp | | | | | | | | | | | |
| copolymer | | | | | | | | | | | |
| Beeswax White | | | | | | | 14.0 | | | | 2.0 |
| Cera Alba | | | | | | | | | | | |
| Benzyl Alcohol | | | | | | | 0.5 | | | | |
| Benzyl Alcohol | | | | | | | | | | | |
| Biotive L-Arginine | | 1.5 | | | | | | 1.0 | | | |
| Arginine | | | | | | | | | | | |
| Carbopol ETD 2050 | | 0.3 | | | | | | | | | |
| Polymer | | | | | | | | | | | |
| Carbomer | | | | | | | | | | | |
| Carbopol Ultrez 21 | | | | | | | | | 0.2 | | |
| Acrylates/c10-30 Alkyl | | | | | | | | | | | |
| Acrylate Crosspolymer | | | | | | | | | | | |
| Carnauba Wax LC | | | | | | | 6.0 | | | | |
| organic | | | | | | | | | | | |
| Copernicia Cerifera | | | | | | | | | | | |
| (Carnuba) Wax | | | | | | | | | | | |
| Carvacrol | | | | | | | | | | 0.1 | |
| Cetiol Sensoft | | 4.0 | | | | | | | | | |
| Propylheptyl caprylate | | | | | | | | | | | |
| Citric acid 10% solution | | | | 0.5 | | | | | | 0.1 | |
| Aqua. Citric acid | | | | | | | | | | | |
| Cocoa Butter | | | | | | | 3.0 | | | | 1.0 |
| Theobroma Cacao (Cocoa) | | | | | | | | | | | |
| Seed Butter | | | | | | | | | | | |
| Coconut oil | | | | | | | 1.0 | | | | |
| Cocos Nucifera (Coconut) | | | | | | | | | | | |
| Oil | | | | | | | | | | | |
| Colour | | | | | 0.4 | | | | | | |
| Colour | | | | | | | | | | | |
| Comperlan ® IP | | | | 1.0 | | | | | | | |
| Cocamide MIPA | | | | | | | | | | | |
| Crinipan AD | | | | | 0.2 | | | | | | |
| Climbazole | | | | | | | | | | | |
| Crinipan PMC Green | 0.2 | 0.5 | 1.0 | 0.3 | 0.1 | 0.2 | 0.5 | 0.5 | 0.1 | 0.3 | 0.2 |
| Propanediol Caprylate | | | | | | | | | | | |
| Cutina GMS V | | | | | | | | | 1.0 | | |
| Glyceryl Stearate | | | | | | | | | | | |
| Cutina HR Powder | | | | | | | | | | | 1.0 |
| Hydrogenated Castor Oil | | | | | | | | | | | |
| Cyclomethicone | | | | | | | | | | 0.2 | |
| Cyclomethicone | | | | | | | | | | | |
| Dehyquart A CA | | | 0.5 | | | | | | | | |
| Cetrimonium Chloride | | | | | | | | | | | |
| Dehyquart SP | | | 4.0 | | | | | | | | |
| Quaternium-52 | | | | | | | | | | | |
| Dehyton PK 45 | | | | 8.0 | 8.0 | | | | | | |
| Cocamidopropyl Betaine | | | | | | | | | | | |
| Dermofeel GO Soft | | | | | | | | | | | 2.5 |
| Polyglyceryl-2 | | | | | | | | | | | |
| Sesquioleate | | | | | | | | | | | |
| Dermofeel sensolv | | | | | | | | | | | 7.0 |
| Isoamyl Laurate | | | | | | | | | | | |
| Dermosoft 1388 eco | | | | | | | | | | | 3.0 |
| Glycerin. Aqua. Sodium | | | | | | | | | | | |
| Levulinate. Sodium Anisate | | | | | | | | | | | |
| Dimethicone Dimethicone | | | 0.1 | | | | | | | | |
| Dissolvine GL-47S | | | 0.2 | | | | | | | | |
| Tetrasodium Glutamate | | | | | | | | | | | |
| Diacetate | | | | | | | | | | | |
| Dracorin CE | 2.0 | | 1.0 | | | 3.0 | | | | | |
| Glyceryl Stearate Citrate | | | | | | | | | | | |
| Dracorin GOC | | | | | | 1.5 | | | 2.0 | | |
| Glyceryl Oleate Citrate. | | | | | | | | | | | |
| Caprylic/Capric | | | | | | | | | | | |
| Triglyceride | | | | | | | | | | | |
| DragoCalm | | | | | | | | | 1.5 | | |
| Aqua. Glycerin. Avena | | | | | | | | | | | |
| sativa kernel extract | | | | | | | | | | | |
| Dragosantol 100 | | | | | | | | | | 0.1 | |
| Bisabolol | | | | | | | | | | | |
| Dragosine | 0.1 | | | | | | | 0.2 | | | |
| Carnosine | | | | | | | | | | | |
| Dragoxat 89 | 2.0 | | | | | 3.0 | | | 3.0 | | |
| Ethylhexyl Isononanoate | | | | | | | | | | | |
| EcoPres XC | | | 0.3 | | 0.1 | | | | | 0.6 | |
| Xylityl Caprylate | | | | | | | | | | | |
| EDTA BD | | 0.1 | | | | 0.1 | | 0.1 | | | |
| Disodium EDTA | | | | | | | | | | | |
| Emulgin B2 | | | | | | | | | | 2.5 | |
| Ceteareth-20 | | | | | | | | | | | |
| Emulsiphos | | 2.5 | | | | | | | | | |
| Potassium Cetyl | | | | | | | | | | | |
| Phosphate. Hydrogenated | | | | | | | | | | | |
| Palm Glycerides | | | | | | | | | | | |
| Estearina L2SM GS | | | | 0.5 | | | | | | | |
| Stearic Acid. Palmitic Acid | | | | | | | | | | | |
| Ethanol | | | | | | | | | | | 5.0 |
| Ethanol | | | | | | | | | | | |
| Euxyl K300 | | | | | | | | | 0.6 | | |
| Methyl-. Butyl-. Ethyl-. | | | | | | | | | | | |
| Propyl. Isobutylparaben. | | | | | | | | | | | |
| Phenoxyethanol | | | | | | | | | | | |
| Floraesters K-100 ® | | | | | | | | 1.0 | | | |
| Jojoba Hydrolyzed jojoba | | | | | | | | | | | |
| esters. Jojoba esters. Aqua | | | | | | | | | | | |
| PO1 or, respectively, | 0.3 | 1.0 | 0.5 | 1.5 | 1.0 | 0.2 | 0.5 | 0.4 | 0.2 | 0.1 | 0.4 |
| PO2 | | | | | | | | | | | |
| Parfum | | | | | | | | | | | |
| Frescolat ML cryst | | 0.5 | | | 1.0 | | | | | | |
| Menthyl Lactate | | | | | | | | | | | |
| Frescolat Plus Menthol. | | | 0.3 | | | | | | | | |
| Menthyl Lactate | | | | | | | | | | | |
| Frescolat X-Cool Menthyl | | | | | | | | 0.8 | | | |
| Ethylamido Oxalate | | | | | | | | | | | |
| Glucotain Care | | | | 2.0 | | | | | | | |
| Cocoyl Methyl Glucamide | | | | | | | | | | | |
| Glycerin | 2.0 | 1.0 | | | | 4.0 | | | 2.5 | 3.0 | 7.0 |
| Glycerin | | | | | | | | | | | |
| Hemp Oil | 1.0 | | | | | | | | | | |
| Cannabis Sativa Seed Oil | | | | | | | | | | | |
| Hydrolex E | | | | | | | | | | 0.3 | |
| Ethylhexylglycerin | | | | | | | | | | | |
| Hydrolite 5 Pentylene | | | | | | | 2.0 | | | 3.0 | |
| Glycol | | | | | | | | | | | |
| Hydrolite 6 | | | | | 1.0 | | | 0.5 | | | |
| 1.2-Hexanediol | | | | | | | | | | | |
| Hydrolite 7 green 1.2- | | 1.0 | | | 1.0 | | | 2.0 | | | |
| Heptanediol | | | | | | | | | | | |
| Hydrolite CG | | | | | | | 0.5 | | | 1.0 | 0.3 |
| Caprylyl Glycol | | | | | | | | | | | |
| Hydroviton ® PLUS 2290 | | | | | | | | | | 1.5 | |
| Aqua. Pentylene glycol. | | | | | | | | | | | |
| Glycerin. Fructose. Urea. | | | | | | | | | | | |
| Citric acid. Sodium | | | | | | | | | | | |
| hydroxide. Maltose. | | | | | | | | | | | |
| Sodium pca. Sodium | | | | | | | | | | | |
| chloride. Sodium lactate. | | | | | | | | | | | |
| Trehalose. Allantoin. | | | | | | | | | | | |
| Sodium hyaluronate | | | | | | | | | | | |
| Glucose | | | | | | | | | | | |
| ImerCare ® Pharma 00T | | | | | | | 2.0 | | | | |
| Talc | | | | | | | | | | | |
| Isoadipate | | 7.5 | | | | | | | | | |
| Diisopropyl Adipate | | | | | | | | | | | |
| Isodragol | | | | | | | | | 4.0 | | |
| Triisononanoin | | | | | | | | | | | |
| Isopropyl Myristate | | | | | | | | | | 0.5 | |
| Isopropyl myristate | | | | | | | | | | | |
| Isopropyl Palmitate | | | | | | | | | | 3.0 | |
| Isopropyl Palmitate | | | | | | | | | | | |
| Jaguar C-162 | | | 0.3 | | | | | | | | |
| Hydroxypropyl guar | | | | | | | | | | | |
| hydroxypropyltrimonium | | | | | | | | | | | |
| chloride | | | | | | | | | | | |
| Jojoba Oil | | | | | | | | | | 0.5 | |
| Simmondsia Chinensis | | | | | | | | | | | |
| Seed Oil | | | | | | | | | | | |
| Lanette E | | 0.8 | | | | | | | | | |
| Sodium cetearyl sulfate | | | | | | | | | | | |
| Lanette O | 5.0 | 1.5 | | 2.0 | | | | | 1.5 | 2.5 | |
| Cetearyl Alcohol | | | | | | | | | | | |
| Lara Care A-200 | | | 0.5 | | | | | | | | |
| Galactoarabinan | | | | | | | | | | | |
| Lexgard Naturals MB | | | | | | | | | | | 1.0 |
| Glyceryl Caprylate (and) | | | | | | | | | | | |
| Glyceryl Undecylenate | | | | | | | | | | | |
| Magnesium Stearate | | | | | | | | | | | 0.7 |
| Magnesium Stearate | | | | | | | | | | | |
| Mango Butter | | | | | | | Ad | | | | |
| Mangifera indica Seed | | | | | | | 100 | | | | |
| Butter | | | | | | | | | | | |
| Mineral oil Paraffinum | | | | | | | | | | 4.0 | |
| Liquidum | | | | | | | | | | | |
| Nativacare 5600 | | | | | 2.0 | 1.5 | | | | | |
| Zea Mays (Corn Starch) | | | | | | | | | | | |
| Natriquest E30 | | | | | 0.3 | | | | | | |
| Trisodiumn Ethylendiamine | | | | | | | | | | | |
| Disuccinate | | | | | | | | | | | |
| Neo-Heliopan 303 | | | | | | | | 10.0 | | | |
| Octocrylene | | | | | | | | | | | |
| Neo-Heliopan 357 Butyl | | 4.0 | | | | | | 3.0 | | | |
| Methoxydibenzoylmethane | | | | | | | | | | | |
| Neo-Heliopan BMT Bis- | | 3.0 | | | | | | 1.5 | | | |
| Ethylhexyloxyphenol | | | | | | | | | | | |
| Methoxypenyl Triazine | | | | | | | | | | | |
| Neo-Heliopan HMS | | | | | | | | 2.5 | | | |
| Homosalate | | | | | | | | | | | |
| Neo-Heliopan Hydro | | 3.0 | | | | | | 2.0 | | | |
| Phenylbenzimidazole | | | | | | | | | | | |
| sulfonic acid | | | | | | | | | | | |
| Neo-Heliopan OS | | 5.0 | | | | | | 5.0 | | | |
| Ethylhexyl Salicylate | | | | | | | | | | | |
| Nonanediol | | | | 0.3 | | | | | | | |
| 1.2-Nonanediol | | | | | | | | | | | |
| Panthenol | | | | | | | | | 0.5 | | |
| Panthenol | | | | | | | | | | | |
| Passion Fruit oil | 1.0 | | | | | | | | | | |
| Passiflora Edulis Seed Oil | | | | | | | | | | | |
| PCL Liquid 100 Cetearyl | | | 0.3 | | | 2.0 | | | | | |
| Ethylhexanoate | | | | | | | | | | | |
| PCL Liquid | | | | | | | | | 7.0 | | |
| Cetearyl Ethylhexanoate. | | | | | | | | | | | |
| Isopropyl Myristate | | | | | | | | | | | |
| PCL Solid | | | 3.0 | | | | | | 2.5 | | |
| Stearyl Heptanoate. | | | | | | | | | | | |
| Stearyl Caprylate | | | | | | | | | | | |
| Phenoxyethanol | | 0.5 | | | | | | | | | |
| Phenoxyethanol | | | | | | | | | | | |
| Phytoconcentrole Carrot | | | | | | | | 0.3 | | | |
| Helianthus Annuus Seed | | | | | | | | | | | |
| Oil. Beta-Carotene. | | | | | | | | | | | |
| Daucus Carota Sativa Root | | | | | | | | | | | |
| Extract | | | | | | | | | | | |
| Phytoconcentrole Hemp | | | | | | | 0.5 | | | | |
| CBD | | | | | | | | | | | |
| Cannabis Sativa Seed Oil. | | | | | | | | | | | |
| Cannabidiol. Tocopherol | | | | | | | | | | | |
| Plantacare 2000 UP decyl | | | | | 4.0 | | | | | | |
| Glucoside | | | | | | | | | | | |
| Potassium Sorbate | | | | | | | | | 0.1 | | |
| Potassium Sorbate | | | | | | | | | | | |
| Propylene Glycol | | 3.0 | | | 2.0 | | | | | | |
| Propylene Glycol | | | | | | | | | | | |
| RetinoPeptide 189 | 1.0 | | | | | | | | | | |
| Glycerin. Pentylene Glycol. | | | | | | | | | | | |
| Water. Myristoyl | | | | | | | | | | | |
| Nonapeptide-3 | | | | | | | | | | | |
| Retinyl Palmitate Retinyl | | | | | | 0.1 | | | | | |
| Palmitate | | | | | | | | | | | |
| Rice Bran Wax Oryza | | | | | | | 6.0 | | | | |
| Sativa (Rice) Bran Wax | | | | | | | | | | | |
| Sensocel 10 | | | | | | | | 3.0 | | | |
| Cellulose | | | | | | | | | | | |
| Shea Butter (Organic) | | 0.3 | | | | | | | | | 1.0 |
| Butyrospermum Parkii | | | | | | | | | | | |
| Butter | | | | | | | | | | | |
| Sodium Gluconate | | | | 0.1 | | | | 0.1 | | | |
| Sodium Gluconate | | | | | | | | | | | |
| Sodium Hydroxide 10% | | 2.0 | | | 2.2 | | | 0.2 | 0.5 | | |
| solution | | | | | | | | | | | |
| Aqua. Sodium Hydroxide | | | | | | | | | | | |
| Softigen Pura | | | | | | | | | | | 5.0 |
| Olus Oil | | | | | | | | | | | |
| Squalane | | | | | | | | | | | 10.0 |
| Squalane | | | | | | | | | | | |
| Super Hartolan Lanolin | | | | | | | | | | 0.2 | |
| Alcohol | | | | | | | | | | | |
| Sweet Almond Oil | | | | | | | 23.5 | | | | |
| Prunus Amygdalus Dulcis | | | | | | | | | | | |
| (Sweet Almond) oil | | | | | | | | | | | |
| SymBright ® 2036 | 0.2 | | | | | | | | | | |
| Sclareolide | | | | | | | | | | | |
| SymBronze | | | | 1.0 | | | 0.7 | | | | |
| Caprylic/Capric | | | | | | | | | | | |
| Triglyceride. Isochrysis | | | | | | | | | | | |
| Galbana Extract | | | | | | | | | | | |
| SymCalmin | | | 0.3 | | | | | | | | |
| Butylene Glycol. Pentylene | | | | | | | | | | | |
| Glycol. Hydroxyphenyl | | | | | | | | | | | |
| Propamidobenzoic Acid | | | | | | | | | | | |
| SymCare O | | | | | | | | 2.0 | | | |
| Hexyldecanol. Pentylene | | | | | | | | | | | |
| Glycol. 4-t- | | | | | | | | | | | |
| Butylcylohexanol. | | | | | | | | | | | |
| Bisabolol. | | | | | | | | | | | |
| Cetylhydroxyproline | | | | | | | | | | | |
| Palmitamide. | | | | | | | | | | | |
| Hydroxyphenyl | | | | | | | | | | | |
| Propamidobenzoic acid. | | | | | | | | | | | |
| Stearic acid. Brassica | | | | | | | | | | | |
| campestris | | | | | | | | | | | |
| (Rapeseed)Sterols. | | | | | | | | | | | |
| Zingiber officinale | | | | | | | | | | | |
| (Ginger)Root extract | | | | | | | | | | | |
| SymCare W2 | | | | | | | | | | 1.5 | |
| PEG-40 Hydrogenated | | | | | | | | | | | |
| Castor Oil. Trideceth-9. | | | | | | | | | | | |
| Pentylene Glycol. 4-t- | | | | | | | | | | | |
| Butylcyclohexa-nol. | | | | | | | | | | | |
| Propylene Glycol. Water. | | | | | | | | | | | |
| Hydroxyphenyl | | | | | | | | | | | |
| Propamidobenzoic Acid | | | | | | | | | | | |
| SymClariol | | | | | 0.5 | | | | | | |
| Decylene Glycol | | | | | | | | | | | |
| SymControl Care Aqua. | | | | | | | | | | 1.0 | |
| Glycerin. Tetraselmis | | | | | | | | | | | |
| Suecica Extract | | | | | | | | | | | |
| SymControl Scalp Aqua. | | | 0.7 | | | | | | | | |
| Glycerin. Mannitol. | | | | | | | | | | | |
| Tetraselmis Suecica | | | | | | | | | | | |
| Extract | | | | | | | | | | | |
| SymDecanox HA | | 1.0 | | | | | 1.0 | | | | 0.5 |
| Caprylic/Capric | | | | | | | | | | | |
| Triglyceride. | | | | | | | | | | | |
| Hydroxymethoxyphenyl | | | | | | | | | | | |
| Decanone | | | | | | | | | | | |
| SymDeo B125 | | | | | | | | 0.5 | | | |
| 2-methyl 5- | | | | | | | | | | | |
| cyclohexylpentanol | | | | | | | | | | | |
| SymDeo Plus | | | | | | | | 0.3 | | | |
| Lauryl alcohol. | | | | | | | | | | | |
| Phenoxyethanol. 2- | | | | | | | | | | | |
| benzylheptanol. Decylene | | | | | | | | | | | |
| glycol | | | | | | | | | | | |
| SymDetox 1711 3.3.5- | | | | | | 0.5 | | | | | |
| Trimethylcyclohexyl | | | | | | | | | | | |
| Succinate Dimethylamide | | | | | | | | | | | |
| SymDiol 68 | | | 0.5 | | | 0.5 | | | | | |
| 1.2-Hexanediol. Caprylyl | | | | | | | | | | | |
| glycol | | | | | | | | | | | |
| SymDStress 100 | | | | | | | | 0.3 | | | |
| Bisabolol. Cannabidiol | | | | | | | | | | | |
| SymEffect Sun | | | | | | | | 1.5 | | | |
| Cera Alba. Sodium | | | | | | | | | | | |
| Stearoyl Lactylate | | | | | | | | | | | |
| SymFinity 1298 | 0.1 | | | | | | | | | | |
| Echinacea Purpurea | | | | | | | | | | | |
| Extract | | | | | | | | | | | |
| SymGlucan | 1.0 | | | | | | | | | 2.0 | |
| Aqua. Glycerin. 1.2- | | | | | | | | | | | |
| Hexanediol. Caprylyl | | | | | | | | | | | |
| Glycol. Beta-Glucan | | | | | | | | | | | |
| SymGuard CD | | | | | | | | 0.5 | | | |
| Phenylpropanol. O-cymen- | | | | | | | | | | | |
| 5-ol. Decylene glycol | | | | | | | | | | | |
| SymHair Force 1631 | | | | | 1.0 | | | | | | |
| Pentylene Glycol. | | | | | | | | | | | |
| Isochrysis Galbana Extract | | | | | | | | | | | |
| SymHair Restore | | | 1.0 | | | | | | | | |
| Glycerin. Triticum vulgare | | | | | | | | | | | |
| protein. Aqua | | | | | | | | | | | |
| SymHair Shield | | | | | 1.0 | | | | | | |
| Pentylene Glycol. Aqua. | | | | | | | | | | | |
| Glycerin. Triticum vulgare | | | | | | | | | | | |
| bran extract. 1.2- | | | | | | | | | | | |
| Hexanediol. Capryly glycol | | | | | | | | | | | |
| SymLift | 1.5 | | | | | 5.0 | | | | | |
| Aqua. Trehalose. Glycerin. | | | | | | | | | | | |
| Pentylene Glycol. Beta- | | | | | | | | | | | |
| Glucan. Hordeum vulgare | | | | | | | | | | | |
| seed extract. sodium | | | | | | | | | | | |
| hyluronate. 1.2- | | | | | | | | | | | |
| Hexanediol. Caprylyl | | | | | | | | | | | |
| Glycol | | | | | | | | | | | |
| SymLite G8 | 0.3 | | 0.2 | | | | | | 0.2 | | |
| Glyceryl Caprylate | | | | | | | | | | | |
| SymMatrix Maltodextrin. | | 0.2 | | | | | | | | | |
| Rubus Fruticosus Leaf | | | | | | | | | | | |
| Extract | | | | | | | | | | | |
| SymMollient PDCC | 3.0 | | | | | | | 2.0 | | | |
| Propanediol | | | | | | | | | | | |
| Dicaprylate/Caprate | | | | | | | | | | | |
| SymMollient S green | 2.0 | 1.0 | | 3.0 | | 2.0 | | | | | |
| Cetearyl Nonanoate | | | | | | | | | | | |
| SymMollient W/S | | | | 3.0 | | | | | | | |
| Trideceth-9. PEG-5 | | | | | | | | | | | |
| Isononanoate. Aqua | | | | | | | | | | | |
| SymOcide PH | | | | 1.0 | | | | | | | |
| Phenoxyethanol. | | | | | | | | | | | |
| Hydroxyacetophenone. | | | | | | | | | | | |
| Caprylyl Glycol | | | | | | | | | | | |
| SymOcide PS | | | 2.0 | | | | | | | | |
| Phenoxyethanol. Decylene | | | | | | | | | | | |
| Glycol. 1.2-Hexanediol | | | | | | | | | | | |
| SymOleo ® Vita7 Glycine | | | | 1.0 | | | | | | | |
| soja oil. Gossypium | | | | | | | | | | | |
| herbaceum seed oil. | | | | | | | | | | | |
| mangifera idica seed | | | | | | | | | | | |
| butter. olea europaea | | | | | | | | | | | |
| fruitoil. persea gratissima | | | | | | | | | | | |
| oil. prununs amygdalus | | | | | | | | | | | |
| dulcis (sweet almond) oil. | | | | | | | | | | | |
| Theobroma cacao seed | | | | | | | | | | | |
| butter | | | | | | | | | | | |
| SymPeptide 225 | | 1.0 | | | | | | | | | |
| Glycerin. Aqua. Myristoyl | | | | | | | | | | | |
| Pentapeptide-11 | | | | | | | | | | | |
| SymRadiance 377 | 0.1 | | | | | | | | | | |
| Phenylethyl Resorcinol | | | | | | | | | | | |
| SymReboot L19 | | | | | | 0.2 | 0.3 | | | | |
| Maltodextrin. Lactobacillus | | | | | | | | | | | |
| Ferment | | | | | | | | | | | |
| SymRelief 100 Bisabolol. | | | | | | 0.1 | | | | | |
| Zingiber officinale root | | | | | | | | | | | |
| extract | | | | | | | | | | | |
| SymRenew ™ HPR | | | | | | | 1.0 | | | | |
| Diisopropyl Adipate. | | | | | | | | | | | |
| Hydroxypinacolone | | | | | | | | | | | |
| Retinoate. Tocopherol | | | | | | | | | | | |
| SymRepair 100 | | | | | | | | | 1.0 | | |
| Hexyldecanol. Bisabolo. | | | | | | | | | | | |
| Cetylhydroxyproline | | | | | | | | | | | |
| Palmitade. Stearic Acid. | | | | | | | | | | | |
| Brassica Campestris | | | | | | | | | | | |
| (Rapeseed) Sterols | | | | | | | | | | | |
| SymSave H | | 0.6 | | | 0.5 | 0.5 | | 0.5 | | | |
| Hydroxyacetophenone | | | | | | | | | | | |
| SymSitive 1609 | | | | | | | | | 1.0 | | |
| Pentylene Glycol. 4-t- | | | | | | | | | | | |
| Butylcyclohexanol | | | | | | | | | | | |
| SymSoft Scalp Trideceth- | | | | | 1.0 | | | | | | |
| 9 (and) PEG-5 | | | | | | | | | | | |
| Ethylhexanoate (and) | | | | | | | | | | | |
| Water (and) | | | | | | | | | | | |
| Hydroxyphenyl | | | | | | | | | | | |
| Propamidobenzoic Acid | | | | | | | | | | | |
| SymSol PF-3 | | | | | | | | 1.5 | | | |
| Aqua. Pentylene Glycol. | | | | | | | | | | | |
| Sodium Lauryl | | | | | | | | | | | |
| Sulfoacetate. Sodium | | | | | | | | | | | |
| Oleoyl Sarcosinte. Sodium | | | | | | | | | | | |
| Chloride. Sodium Oleate | | | | | | | | | | | |
| SymUrban Benzylidene | | 0.3 | | | | | | | | | |
| Dimethoxydimetylindanone | | | | | | | | | | | |
| SymVital AR 3040 | | 0.2 | | | | | | | | | |
| Zinbiber officinale root | | | | | | | | | | | |
| extract | | | | | | | | | | | |
| Tagat L2 | | | | | | | | | | 2.5 | |
| PEG-20 Glyceryl Laurate | | | | | | | | | | | |
| Tapioca Pure Tapioca | | 2.0 | | | | | | | | | |
| Starch | | | | | | | | | | | |
| Tegin ® BL 315 | | | | 1.5 | | | | | | | |
| Glycol Distearate | | | | | | | | | | | |
| Tego Cosmo P813 | | | | | | | 0.5 | | | | |
| Polyglyceryl-3 Caprylate | | | | | | | | | | | |
| Tegosoft TN | | 11.0 | | | | | | | | | |
| C12-15 alkyl benzoate | | | | | | | | | | | |
| Texapon N70 | | | | 16.0 | 35.0 | | | | | | |
| Sodium Laureth Sulfate | | | | | | | | | | | |
| Tocopheryl Acetate | | | | | | 0.2 | | | | | 0.3 |
| Tocopheryl Acetate | | | | | | | | | | | |
| Uvasorb HEB | | 2.0 | | | | | | | | | |
| Diethylhexyl butamido | | | | | | | | | | | |
| triazone | | | | | | | | | | | |
| Vegetable oil triglyceride | 6.0 | | | | | | | | | | |
| Caprylic/Capric | | | | | | | | | | | |
| Triglyceride | | | | | | | | | | | |
| Water | Ad | Ad | Ad | Ad | Ad | Ad | | Ad | Ad | Ad | Ad |
| Aqua | 100 | 100 | 100 | 100 | 100 | 100 | | 100 | 100 | 100 | 100 |
| Xanthan Gum | 0.4 | 0.2 | | | | 0.5 | 0.2 | 0.1 | 0.1 | | |
| Xanthan Gum | | | | | | | | | | | |
| Xiameter ® PMX-0345 | | | | | | | | | 0.5 | | |
| Cyclosiloxane | | | | | | | | | | | |
| Cyclopentasiloxane. | | | | | | | | | | | |
| Cyclohexasiloxane | | | | | | | | | | | |
| Zinc omadine | | | | | 0.3 | | | | | | |
| Zinc Pyrithione | | | | | | | | | | | |
| Zinc Sulfate | | | | | | | | | | | 1.0 |
| Heptahydrate | | | | | | | | | | | |
| Zinc Sulfate | | | | | | | | | | | |

TABLE 16
Cosmetic formulations 12 to 20 (amounts in % b.w.)

| | Amounts [wt.-%] | | | | | | | | |
|---|---|---|---|---|---|---|---|---|---|
| Ingredients INCI name | 12 | 13 | 14 | 15 | 16 | 17 | 18 | 19 | 20 |
| Abil 350 | | | | 2.0 | | | | | |
| Dimethicone | | | | | | | | | |
| Allantoin | | | | 0.2 | | | | | |
| Allantoin | | | | | | | | | |
| Aloe Vera Gel Concentrate | | 0.3 | | | | | | | |
| 10/1 | | | | | | | | | |
| Aloe Barbadensis Leaf Juice | | | | | | | | | |
| Aminat G Glycerin and Ethyl | | | | | | | | 0.8 | |
| Lauroyl Arginate HCl | | | | | | | | | |
| AMISOFT ® CS-11/CS-11(F) | | | | | 0.5 | | | | |
| SODIUM COCOYL | | | | | | | | | |
| GLUTAMATE | | | | | | | | | |
| Anisic Acid | | | | 0.4 | | | | | |
| Anisic Acid | | | | | | | | | |
| Carbopol Ultrez-10 | | | | 0.1 | | | | | |
| Carbomer | | | | | | | | | |
| Carbopol ® Aqua SF-1 | | | | | 10.0 | | | | |
| Polymer | | | | | | | | | |
| ACRYLATES COPOLYMER | | | | | | | | | |
| Ceramide Bio | | | | 0.1 | | | | | |
| Cetylhydroxyproline | | | | | | | | | |
| Palmitamide | | | | | | | | | |
| Citric acid 10% solution | 1.7 | | | 0.4 | | | | | |
| Aqua, Citric acid | | | | | | | | | |
| Coconut oil refined | 10.0 | | | | | | | | |
| Cocos Nucifera (Coconut) Oil | | | | | | | | | |
| Colour | | 0.04 | | | | | | | |
| Covathick 2009 | 0.5 | | | | | | | | |
| Microcrystalline Cellulose, | | | | | | | | | |
| Cellulose Gum | | | | | | | | | |
| Crinipan PMC green | 1.0 | 0.2 | 1.2 | 0.5 | 0.1 | 0.4 | 1.5 | 0.3 | 0.6 |
| Propanediol Caprylate | | | | | | | | | |
| Cutina GMS V | 1.0 | | | 2.0 | | | | | |
| Glyceryl Stearate | | | | | | | | | |
| Dermofeel BGC | 5.0 | | | | | | | | |
| Butylene Glycol | | | | | | | | | |
| Dicaprylate/Dicarate | | | | | | | | | |
| Dermofeel PA | | | | | 0.1 | | | | |
| Phytic Acid; Aqua | | | | | | | | | |
| Dermofeel sensolv MB | | | | | | | | 4.5 | |
| Isoamyl Laurate | | | | | | | | | |
| Dermosoft 1388 eco | 3.0 | | | | | | | | |
| Glycerin, Aqua, Sodium | | | | | | | | | |
| Levulinate, Sodium Anisate | | | | | | | | | |
| Dow Corning FZ-3196 | | | 4.0 | | | | | | |
| Volatile | | | | | | | | | |
| Caprylyl Methicone | | | | | | | | | |
| Dracorin 100 SEP | | | 6.0 | | | | | | |
| Glyceryl Stearate, PEG-100 | | | | | | | | | |
| Stearate | | | | | | | | | |
| Dracorin CE | | 5.0 | | | | | | | |
| Glyceryl Stearate Citrate | | | | | | | | | |
| Drago-Oat-Active | 2.0 | | | | | | | | |
| Water (Aqua), Butylene | | | | | | | | | |
| Glycol, Avena Sativa (Oat) | | | | | | | | | |
| Kernel Extract | | | | | | | | | |
| Dragosantol | 0.2 | | 0.05 | | | | | | 0.1 |
| Bisabolol | | | | | | | | | |
| Dragosine | | | | | | 0.2 | | | |
| Carnosine | | | | | | | | | |
| Dragoxat 89 | | | | | | 2.0 | | | |
| Ethylhexyl Isononanoate | | | | | | | | | |
| EDTA BD | | 0.1 | 0.1 | | | 0.1 | | | |
| Disodium EDTA | | | | | | | | | |
| Emulsiphos | | | | 2.0 | | 2.5 | | | |
| Potassium Cetyl Phosphate, | | | | | | | | | |
| Hydrogenated Palm | | | | | | | | | |
| Glycerides | | | | | | | | | |
| Ethanol | 5.0 | | | | | | Ad | | 30.0 |
| Ethanol | | | | | | | 100 | | |
| Eumulgin B2 | | | 6.0 | | | | | | |
| Ceteareth-20 | | | | | | | | | |
| Eutanol G | | | 8.0 | | | | | | |
| Octyldecanol | | | | | | | | | |
| Euxyl K 903 | | | | 0.3 | | | | | |
| Benzyl Alcohol, Benzoic Acid, | | | | | | | | | |
| Dehydroacetic Acid, | | | | | | | | | |
| Tocopherol | | | | | | | | | |
| Extrapone Arnica GW | | | | 1.0 | | | | | |
| Glycerin, Water (Aqua), Arnica | | | | | | | | | |
| Montana Flower Extract | | | | | | | | | |
| Extrapone Green Tea GW | | | | 0.2 | | | | | |
| Glycerin, Water (Aqua), | | | | | | | | | |
| Camellia Sinensis Leaf Extract | | | | | | | | | |
| Extrapone Peppermint | | | | | | | 0.2 | | |
| Aqua, Propylene Glycol, | | | | | | | | | |
| Mentha Piperita Leaf extract | | | | | | | | | |
| Extrapone Rose Quartz GW | | | 1.0 | | | | | | |
| Aqua, Glycerin, Xanthan Gum, | | | | | | | | | |
| Quartz Powder | | | | | | | | | |
| Farmal MS6135 | | | | | | 2.0 | | | |
| Calcium Starch | | | | | | | | | |
| Octenylsuccinate | | | | | | | | | |
| Farnesol | | | | | | | | | 0.5 |
| Farnesol | | | | | | | | | |
| Farnesol Plus | | | | | | | | 0.5 | |
| Phenoxyethanol, Farnesol, | | | | | | | | | |
| Glyceryl Laurate | | | | | | | | | |
| Floraesters K-100 ® | | | | | | 0.5 | | | |
| Jojoba Hydrolyzed jojoba | | | | | | | | | |
| esters, Jojoba esters, Aqua | | | | | | | | | |
| PO1 or, respectively, PO2 | 0.3 | 0.5 | 1.5 | 0.2 | 0.8 | 0.5 | 1.2 | 0.6 | 1.0 |
| Parfum | | | | | | | | | |
| Frambinone Methyl Ether | | | | | | | | 0.4 | |
| Raspberry Ketone Methylether | | | | | | | | | |
| Frambinone | 0.5 | | | | | | | | |
| Raspberry Ketone | | | | | | | | | |
| Frescolat ML | | | 0.5 | | 0.3 | | 0.5 | | |
| Menthyl Lactate | | | | | | | | | |
| Frescolat Plus | | 0.2 | | | | | | | 0.3 |
| Menthol, Menthyl Lactate | | | | | | | | | |
| Frescolat X-Cool | 1.0 | | | | | | | | |
| Menthyl Ethylamido Oxalate | | | | | | | | | |
| Genuvisco CG-131 | 0.3 | | | | | | | | |
| Carrageenan | | | | | | | | | |
| Glycerin | 3.0 | 3.0 | 5.0 | 2.0 | | | | | |
| Glycerin | | | | | | | | | |
| Hydrolex E | | | | | | | 0.2 | | |
| Ethylhexylglcerin | | | | | | | | | |
| Hydrolite 5 green | | | | | 1.0 | | | 3.0 | |
| 1,2-Pentanediol | | | | | | | | | |
| Hydrolite 6 | | | | | 0.5 | | | | |
| 1,2-Hexanediol | | | | | | | | | |
| Hydrolite 7 green | 0.5 | | | | | 0.5 | 0.3 | | |
| 1,2-Heptanediol | | | | | | | | | |
| Hydrolite CG | | | | | | | | 0.5 | |
| Caprylyl Glycol | | | | | | | | | |
| Hydroviton 24 | | 1.0 | | | | | | | |
| Water, Glycerin, Sodium | | | | | | | | | |
| Lactate, TEA Lactate, Serine, | | | | | | | | | |
| Lactic Acid, Urea, Sorbitol, | | | | | | | | | |
| Sodium Chloride, Lauryl | | | | | | | | | |
| Diethylenedi-aminoglycine, | | | | | | | | | |
| Lauryl Aminopropyl-glycine, | | | | | | | | | |
| Allantoin | | | | | | | | | |
| Irgasan DP 300 | | | | | | | | | 0.1 |
| Triclosan | | | | | | | | | |
| Isodragol | | | | 2.0 | | | | | |
| Triisononanoin | | | | | | | | | |
| Isolan GPS | | | | | | | | 3.5 | |
| Polyglyceryl-4 Diisostearate/ | | | | | | | | | |
| Polyhydroxystearate/ | | | | | | | | | |
| Sebacate | | | | | | | | | |
| Kelogel F | | 0.1 | | | | | | | |
| Gellan Gum | | | | | | | | | |
| Lanette 16 | | 5.0 | 2.0 | | | | | | |
| Cetyl Alcohol | | | | | | | | | |
| Lanette O | | | | 3.0 | | | | | |
| Cetearyl Alcohol | | | | | | | | | |
| Locron P | | | 15.0 | | | | | | |
| Aluminium Chlorohydrate | | | | | | | | | |
| Natrosol 250 HHR | | | | | | | | | 0.3 |
| Hydroxyethyl cellulose | | | | | | | | | |
| Neo PCL wssl, N | | | | 1.0 | | | | | |
| Trideceth-9, PEG-5 | | | | | | | | | |
| Ethylhexanoate, Water | | | | | | | | | |
| Neo-Heliopan 303 | | | | | | 10.0 | | | |
| Octocrylene | | | | | | | | | |
| Neo-Heliopan 357 | | | | | | 4.5 | | | |
| Butyl | | | | | | | | | |
| Methoxydibenzoylmethane | | | | | | | | | |
| Neo-Heliopan BMT | | | | | | 2.5 | | 3.0 | |
| Bis-Ethylhexyloxyphenol | | | | | | | | | |
| Methoxypenyl Triazine | | | | | | | | | |
| Neo-Heliopan HMS | | | | | | 10.0 | | 5.0 | |
| Homosalate | | | | | | | | | |
| Neo-Heliopan OS | | | | | | 5.0 | | 4.0 | |
| Ethylhexyl Salicylate | | | | | | | | | |
| Nerolidol | | | | 0.2 | | | | | |
| Nerolidol | | | | | | | | | |
| Passion Fruit Oil Refined | | 2.0 | | | | | | | |
| Passiflora Edulis Seed Oil | | | | | | | | | |
| PCL Liquid 100 | | | | 5.0 | | | | | |
| Cetearyl Ethylhexanoate | | | | | | | | | |
| PCL Solid | | | | 2.0 | | | | | |
| Stearyl Heptanoate, Stearyl | | | | | | | | | |
| Caprylate | | | | | | | | | |
| Plant oil triglyceride | | 3.0 | | | | | | | |
| Caprylic/Capric Triglyceride | | | | | | | | | |
| Prisorine 3505 | | | | | | 1.5 | | | |
| Isostearic acid | | | | | | | | | |
| Propylene Glycol | | | | 5.0 | | | | | |
| Propylene Glycol | | | | | | | | | |
| Sodium Hydroxide 10% | | 0.2 | 0.5 | 0.3 | 2.0 | | | | |
| solution | | | | | | | | | |
| Aqua, Sodium Hydroxide | | | | | | | | | |
| Softisan 100 | | 6.0 | | | | | | | |
| Hydrogenated Coco- | | | | | | | | | |
| Glycerides | | | | | | | | | |
| Solubilizer | | | | | | | | | 2.0 |
| PEG-40 Hydrogenate castor | | | | | | | | | |
| Oil, Trideceth-9, Water (Aqua) | | | | | | | | | |
| Spectrastat | | | | | | | | 1.0 | |
| Caprylhydroxamic Acid, | | | | | | | | | |
| Caprylyl Glycol, Glycerin | | | | | | | | | |
| SymCalmin | | | | 2.0 | | | | | |
| Butylene Glycol, Pentylene | | | | | | | | | |
| Glycol, Hydroxyphenyl | | | | | | | | | |
| Propamidobenzoic Acid | | | | | | | | | |
| SymClariol | | | | | | | 0.25 | | |
| Decylene Glycol | | | | | | | | | |
| SymDeo B125 | | | 0.3 | | | | | | 0.5 |
| 2-Methyl 5-Cyclohexylpentanol | | | | | | | | | |
| SymDeo MPP | | | | | | | 0.4 | | |
| Dimethyl Phenylbutanol | | | | | | | | | |
| SymDecanox HA | | | | | | 1.0 | | | |
| Caprylic/Capric Triglyceride, | | | | | | | | | |
| Hydroxymethoxyphenyl | | | | | | | | | |
| Decanone | | | | | | | | | |
| SymDiol 68 | | 0.5 | | 1.0 | | | | | |
| 1,2-Hexanediol, Caprylyl | | | | | | | | | |
| glycol | | | | | | | | | |
| SymDStress 100 | 0.1 | | | | | | | | |
| Bisabolol, Cannabidiol | | | | | | | | | |
| SymLite G8 | | | | | | | 0.15 | | 0.1 |
| Glyceryl Caprylate | | | | | | | | | |
| SymMatrix | | | | 0.1 | | | | | |
| Maltodextrin, Rubus | | | | | | | | | |
| Fruticosus Leaf Extract | | | | | | | | | |
| SymMollient PDCC | | | | | | | | | |
| Propanediol | | | | | | 3.5 | | | |
| Dicaprylate/Caprate | | | | | | | | | |
| SymMollient S green | | 1.0 | | | | | | | |
| Cetearyl Nonanoate | | | | | | | | | |
| SymOcide C | | | | 0.1 | | | | | |
| o-cymen-5-ol | | | | | | | | | |
| SymOcide PH | | | | | | 1.4 | | | |
| Phenoxyethanol, | | | | | | | | | |
| Hydroxyacetophenone, | | | | | | | | | |
| Caprylyl Glycol, Aqua | | | | | | | | | |
| SymOcide PS | | | 1.0 | | | | | | |
| Phenoxyethanol, Decylene | | | | | | | | | |
| Glycol, 1,2-Hexanediol | | | | | | | | | |
| SymRelief S | | | | 0.2 | | | | | |
| Bisabolol, | | | | | | | | | |
| Hydroxymethoxyphenyl | | | | | | | | | |
| Decanone | | | | | | | | | |
| SymSave H | | 0.5 | | | 0.5 | | | | |
| Hydroxyacetophenone | | | | | | | | | |
| SymSitive 1609 | | | | 1.5 | | | | | |
| Pentylene Glycol, 4-t- | | | | | | | | | |
| Butylcyclohexanol | | | | | | | | | |
| SymSol PF-3 | | | | | 3.0 | 2.0 | | | |
| Water (Aqua), Sodium Lauryl | | | | | | | | | |
| Sulfoacetate, Pentylene | | | | | | | | | |
| Glycol, Sodium Oleoyl | | | | | | | | | |
| Sarcosinate, Sodium Chloride, | | | | | | | | | |
| Sodium Oleate | | | | | | | | | |
| SymUrban | | | | | | 0.2 | | | |
| Benzylidene | | | | | | | | | |
| Dimethoxydimetylindanone | | | | | | | | | |
| SymVital AR 3040 | | | | | 0.1 | 0.1 | | | |
| Zinbiber officinale root extract | | | | | | | | | |
| Tapioca Pure | | | | | | 1.5 | | | |
| Tapioca Starch | | | | | | | | | |
| Tego Care CG90 | 1.0 | | | | | | | | |
| Cetearyl Glucoside | | | | | | | | | |
| TEGOSOFT DC MB | | | | | | | | 4.0 | |
| Decyl Cocoate | | | | | | | | | |
| Thymol | | 0.1 | | | 0.05 | | | | |
| Thymol | | | | | | | | | |
| Tocopherol | 0.3 | 0.1 | | 0.1 | 0.1 | 0.3 | | 0.5 | |
| Tocopherol | | | | | | | | | |
| Tocopheryl Acetate | | 0.2 | | | | | | | |
| Tocopheryl Acetate | | | | | | | | | |
| Tween 20 | | | 0.3 | | | | | | |
| Polysorbate-20 | | | | | | | | | |
| Uvinul A plus | | | | | | | | 3.0 | |
| Diethylamino Hydroxybenzoyl | | | | | | | | | |
| Hexyl Benzoate | | | | | | | | | |
| Uvinul T150 | | | | | | | | 3.0 | |
| Ethylhexyl Triazone | | | | | | | | | |
| Veegum HS Granules | | | 1.0 | | | | | | |
| Magnesium Aluminium Silicate | | | | | | | | | |
| Verstatil TBO | | | | | | | | 1.0 | |
| Triethylcitrate; Caprylyl Glycol; | | | | | | | | | |
| benzoic Acid | | | | | | | | | |
| Vitacel CS020 | | | 1.0 | | | | | | |
| Cellulose | | | | | | | | | |
| Vivapur CS 032 XV | | | | | | 1.0 | | | |
| Microcrystalline Cellulose, | | | | | | | | | |
| Xanthan Gum | | | | | | | | | |
| Vivapur CS Tex sun | | | | | | 1.0 | | | |
| Microcrystalline Cellulose, | | | | | | | | | |
| Cellulose Gum | | | | | | | | | |
| Water | Ad | Ad | Ad | Ad | Ad | Ad | | Ad | Ad |
| Aqua | 100 | 100 | 100 | 100 | 100 | 100 | | 100 | 100 |
| Xanthan Gum | 0.3 | | | 0.1 | | 0.3 | | | |
| Xanthan Gum | | | | | | | | | |
| Zinc Stearate | | | | | | | | 0.5 | |
| Zinc Stearate | | | | | | | | | |
| Zinc Sulfate Heptahydrate | | | | | | | | 1.2 | |
| Zinc Sulfate | | | | | | | | | |
| Zirkonal L540 | | | | | | | | | 37.0 |
| Aluminum Zirconium | | | | | | | | | |
| Pentachlorohydrate | | | | | | | | | |

1-15. (canceled)

16. A method for enhancing an antimicrobial effect of a first compound, a second compound, or a mixture thereof, the method comprising combining one or more first compounds with one or more second compounds, wherein:
(i) the one or more first compounds are selected from 3-hydroxypropyl caprylate, 3-hydroxypropyl undecylenate, or a combination thereof,
(ii) the one or more second compounds are selected from piroctone olamine, climbazole, zinc pyrithione, salicylic acid, salts of salicylic acid, decylene glycol, o-cymen-5-ol, selenium sulfide, coal tar, sulphur, ketoconazole, benzoic acid, salts of benzoic acid, sorbic acid, salts of sorbic acid, lactic acid, salts of lactic acid, dehydroacetic acid, salts of dehydroacetic acid, undecylenic acid, salts of undecylenic acid, levulinic acid, salts of levulinic acid, anisic acid, salts of anisic acid, phytic acid, salts of phytic acid, itaconic acid, salts of itaconic acid, MEA undecylenate, butyloctanoic acid, salts of butyloctanoic acid, citric acid, salts of citric acid, hydroxyacetophenone, ethylhexylglycerin, caprylhydroxamic acid, salts of caprylhydroxamic acid, undecylenoyl glycine, capryloyl glycine, polyglyceryl-3 caprylate, glyceryl undecylenate, glyceryl caprylate, sorbitan caprylate, glyceryl laurate, phenethyl alcohol, tropolones, 1,2-propanediol, 1,2-butanediol, 1,2-pentanediol, 1,2-hexanediol, 1,2-heptanediol, 1,2-octanediol, 1,2-nonanediol, 1,2-decanediol, 1,2-undecanediol, 1,2-dodecanediol, 1,2-tridecanediol, 2,3-pentanediol, 2,3-hexanediol, 2,3-heptanediol, 2,3-octanediol, 2,3-nonanediol, 2,3-decanediol, 2,3-undecanediol, 2,3-dodecanediol, 2,3-tridecanediol, 1,3-propanediol, 1,3-butanediol, 2-methyl-2,4-pentanediol, caprylyl glycol, farnesol, bisabolol, nerolidol, carvacrol, thymol, phenylpropanol, methylbenzyl alcohol, 2-benzylheptanol, 2-methyl 5-cyclohexylpentanol, dimethyl phenylbutanol, PCA ethyl cocoyl arginate, benzyl alcohol, phenoxyethanol, iodopropynyl butylcarbamate, hydroxyethoxyphenyl butanol, ethyl lauroyl arginate HCl, methylchloroisothiazolinone, methylisothiazolinone, methylparaben and salts thereof, preferably sodium methylparaben, ethylparaben, salts of ethylparaben, propylparaben, salts of propylparaben, isopropylparaben, salts of isopropylparaben, butylparaben, salts of salts of butylparaben, isobutylparaben, salts of isobutylparaben, DMDM hydantoin, diazolidinyl urea, imidazolidinyl urea, chlorphenesin, disodium EDTA, tetrasodium glutamate diacetate, trisodium ethylenediamine disuccinate, methylglycine N,N-diacetic trisodium salt, glucoheptonic sodium salt, ethanoldiglycinic disodium salt, sodium gluconate, tetrasodium iminodisuccinate, trisodium salt of methylglycine diacetic acid, polyaspartic acid sodium salt, phenoxyisopropanol, chlorhexidine, chlorhexidine digluconate, triclocarban, triclosan, O-phenylphenol, 4-hydroxybenzoic acid, TEA-salicylate, benzyl salicylate, hexamidine, hexamidine diisethionate, dichlorobenzyl alcohol, ethyl benzoate, sodium paraben, isothiazolinones, p-chloro-m-cresol, cetrimonium chloride, cetrimonium bromide, benzethonium chloride, alkyltrimonium bromide, alkyltrimonium chloride, benzalkonium chloride, benzalkonium bromide, myristamidopropyl PG-dimonium chloride phosphate, guar hydroxypropyltrimonium chloride, hydroxypropyl guar hydroxypropyltrimonium chloride, polyquaternium-6, polyquaternium-7, polyquaternium-10, polyquaternium-11, polyquaternium-12, polyquaternium-15, methylheptylglycerin, sodium caproyl/lauroyl lactylate, polyglyceryl-10 laurate, glyceryl caprate, polyglyceryl-2 caprate, raspberry ketone, raspberry ketone methyl ether, caprylyl caprylate, coco-caprylate/caprate, PEG-8 caprylate, dipropylene glycol caprylate, propylene glycol caprylate, pyridoxine dicaprylate, glyceryl caprylate/caprate, methyl glucose caprylate/caprate, polyglyceryl-3 caprate/caprylate/succinate, polyglyceryl-2 caprylate, polyglyceryl-3 caprylate, polyglyceryl-4 caprylate, polyglyceryl-6 caprylate, polyglyceryl-caprylate, polyglyceryl-4 caprylate/caprate, polyglyceryl-6 caprylate/caprate, polyglyceryl-10 caprylate/caprate, propanediol dicaprylate/caprate, xylityl caprate/caprylate, xylityl caprylate, xylityl sesquicaprylate, hydroxyethoxyphenyl butanone, or mixtures thereof;
wherein one or more of the first compounds enhance an antimicrobial effect of one or more of the second compounds, and/or one or more of the second compounds enhance an antimicrobial effect of one or more of the first compounds.

17. The method of claim 16, wherein the antimicrobial effect is synergistically enhanced.

18. The method of claim 16, wherein the antimicrobial effect is selected from an antibacterial effect, an antiviral effect, an antifungal effect, or combinations thereof.

19. The method of claim 16, wherein the antimicrobial effect is against one or more microorganisms selected from bacteria, fungi, or a combination thereof.

20. The method of claim 16, wherein the antimicrobial effect is against Staphylococcus, Escherichia, Pseudomonas, Aspergillus, Candida, Pluralibacter, Burkholderia, Bacillus, Enterococcus, Salmonella, or combinations thereof.

21. The method of claim 16, wherein the antimicrobial effect is against Staphylococcus aureus, Escherichia coli, Pseudomonas aeruginosa, Aspergillus brasiliensis, Candida albicans, Pluralibacter gergoviae, Burkholderia cepacia, Bacillus cereus, Enterococcus faecalis, Salmonella enteritidis, or combinations thereof.

22. A method for providing an antimicrobial effect to a product comprising adding to the product an antimicrobial mixture or composition in an amount sufficient to provide the antimicrobial effect to the product, wherein the mixture or composition comprises:
(i) one or more first compounds selected from 3-hydroxypropyl caprylate, 3-hydroxypropyl undecylenate, or a combination thereof;
(ii) one or more second compounds selected from piroctone olamine, climbazole, zinc pyrithione, salicylic acid, salts of salicylic acid, decylene glycol, o-cymen-5-ol, selenium sulfide, coal tar, sulphur, ketoconazole, benzoic acid, salts of benzoic acid, sorbic acid, salts of sorbic acid, lactic acid, salts of lactic acid, dehydroacetic acid, salts of dehydroacetic acid, undecylenic acid, salts of undecylenic acid, levulinic acid, salts of levulinic acid, anisic acid, salts of anisic acid, phytic acid, salts of phytic acid, itaconic acid, salts of itaconic acid, MEA undecylenate, butyloctanoic acid, salts of butyloctanoic acid, citric acid, salts of citric acid, hydroxyacetophenone, ethylhexylglycerin, caprylhydroxamic acid, salts of caprylhydroxamic acid, undecylenoyl glycine, capryloyl glycine, polyglyceryl-3 caprylate, glyceryl undecylenate, glyceryl caprylate, sorbitan caprylate, glyceryl laurate, phenethyl alcohol, tropolones, 1,2-propanediol, 1,2-butanediol, 1,2-pentanediol, 1,2-hexanediol, 1,2-heptanediol, 1,2-octanediol, 1,2-nonanediol, 1,2-decanediol, 1,2-undecanediol, 1,2-dodecanediol, 1,2-tridecanediol, 2,3-pentanediol, 2,3-hexanediol, 2,3-heptanediol, 2,3-octanediol, 2,3-nonanediol, 2,3-decanediol, 2,3-undecanediol, 2,3-dodecanediol, 2,3-tridecanediol, 1,3-propanediol, 1,3-butanediol, 2-methyl-2,4-pentanediol, caprylyl glycol, farnesol, bisabolol, nerolidol, carvacrol, thymol, phenylpropanol, methylbenzyl alcohol, 2-benzylheptanol, 2-methyl 5-cyclohexylpentanol, dimethyl phenylbutanol, PCA ethyl cocoyl arginate, benzyl alcohol, phenoxyethanol, iodopropynyl butylcarbamate, hydroxyethoxyphenyl butanol, ethyl lauroyl arginate HCl, methylchloroisothiazolinone, methylisothiazolinone, methylparaben and salts thereof, preferably sodium methylparaben, ethylparaben, salts of ethylparaben, propylparaben, salts of propylparaben, isopropylparaben, salts of isopropylparaben, butylparaben, salts of salts of butylparaben, isobutylparaben, salts of isobutylparaben, DMDM hydantoin, diazolidinyl urea, imidazolidinyl urea, chlorphenesin, disodium EDTA, tetrasodium glutamate diacetate, trisodium ethylenediamine disuccinate, methylglycine N,N-diacetic trisodium salt, glucoheptonic sodium salt, ethanoldiglycinic disodium salt, sodium gluconate, tetrasodium iminodisuccinate, trisodium salt of methylglycine diacetic acid, polyaspartic acid sodium salt, phenoxyisopropanol, chlorhexidine, chlorhexidine digluconate, triclocarban, triclosan, O-phenylphenol, 4-hydroxybenzoic acid, TEA-salicylate, benzyl salicylate, hexamidine, hexamidine diisethionate, dichlorobenzyl alcohol, ethyl benzoate, sodium paraben, isothiazolinones, p-chloro-m-cresol, cetrimonium chloride, cetrimonium bromide, benzethonium chloride, alkyltrimonium bromide, alkyltrimonium chloride, benzalkonium chloride, benzalkonium bromide, myristamidopropyl PG-dimonium chloride phosphate, guar hydroxypropyltrimonium chloride, hydroxypropyl guar hydroxypropyltrimonium chloride, polyquaternium-6, polyquaternium-7, polyquaternium-10, polyquaternium-11, polyquaternium-12, polyquaternium-15, methylheptylglycerin, sodium caproyl/lauroyl lactylate, polyglyceryl-10 laurate, glyceryl caprate, polyglyceryl-2 caprate, raspberry ketone, raspberry ketone methyl ether, caprylyl caprylate, coco-caprylate/caprate, PEG-8 caprylate, dipropylene glycol caprylate, propylene glycol caprylate, pyridoxine dicaprylate, glyceryl caprylate/caprate, methyl glucose caprylate/caprate, polyglyceryl-3 caprate/caprylate/succinate, polyglyceryl-2 caprylate, polyglyceryl-3 caprylate, polyglyceryl-4 caprylate, polyglyceryl-6 caprylate, polyglyceryl-10 caprylate, polyglyceryl-4 caprylate/caprate, polyglyceryl-6 caprylate/caprate, polyglyceryl-10 caprylate/caprate, propanediol dicaprylate/caprate, xylityl caprate/caprylate, xylityl caprylate, xylityl sesquicaprylate, hydroxyethoxyphenyl butanone, or mixtures thereof.

23. The method of claim 22, wherein one or more of the first compounds synergistically improve an antimicrobial effect of one or more of the second compounds, and/or one or more of the second compounds synergistically improve an antimicrobial effect of one or more of the first compounds.

24. The method of claim 22, wherein the product is a personal care product comprising:
(i) from 0.01 to 10 wt. % of the one or more first compounds, based on a total weight of the personal care product; and/or
(ii) from 0.001 to 20 wt. % of the one or more second compounds, based on the total weight of the personal care product.

25. The method of claim 22, wherein the personal care product is selected from a shampoo, a cleansing product, a body wash, a soap, a wet wipe, an emulsion, a surfactant-based system, a tonic, a gel, a conditioner, a hair butter, a hair wax, or a beard care product.

26. The method of claim 22, wherein the method stabilizes a personal care product and comprises:
(a) providing one or more first compounds and one or more second compounds, wherein:
(i) the one or more first compounds are selected from 3-hydroxypropyl caprylate, 3-hydroxypropyl undecylenate, or a combination thereof,
(ii) the one or more second compounds are selected from piroctone olamine, climbazole, zinc pyrithione, salicylic acid, salts of salicylic acid, decylene glycol, o-cymen-5-ol, selenium sulfide, coal tar, sulphur, ketoconazole, benzoic acid, salts of benzoic acid, sorbic acid, salts of sorbic acid, lactic acid, salts of lactic acid, dehydroacetic acid, salts of dehydroacetic acid, undecylenic acid, salts of undecylenic acid, levulinic acid, salts of levulinic acid, anisic acid, salts of anisic acid, phytic acid, salts of phytic acid, itaconic acid, salts of itaconic acid, MEA undecylenate, butyloctanoic acid, salts of butyloctanoic acid, citric acid, salts of citric acid, hydroxyacetophenone, ethylhexylglycerin, caprylhydroxamic acid, salts of caprylhydroxamic acid, undecylenoyl glycine, capryloyl glycine, polyglyceryl-3 caprylate, glyceryl undecylenate, glyceryl caprylate, sorbitan caprylate, glyceryl laurate, phenethyl alcohol, tropolones, 1,2-propanediol, 1,2-butanediol, 1,2-pentanediol, 1,2-hexanediol, 1,2-heptanediol, 1,2-octanediol, 1,2-nonanediol, 1,2-decanediol, 1,2-undecanediol, 1,2-dodecanediol, 1,2-tridecanediol, 2,3-pentanediol, 2,3-hexanediol, 2,3-heptanediol, 2,3-octanediol, 2,3-nonanediol, 2,3-decanediol, 2,3-undecanediol, 2,3-dodecanediol, 2,3-tridecanediol, 1,3-propanediol, 1,3-butanediol, 2-methyl-2,4-pentanediol, caprylyl glycol, farnesol, bisabolol, nerolidol, carvacrol, thymol, phenylpropanol, methylbenzyl alcohol, 2-benzylheptanol, 2-methyl 5-cyclohexylpentanol, dimethyl phenylbutanol, PCA ethyl cocoyl arginate, benzyl alcohol, phenoxyethanol, iodopropynyl butylcarbamate, hydroxyethoxyphenyl butanol, ethyl lauroyl arginate HCl, methylchloroisothiazolinone, methylisothiazolinone, methylparaben and salts thereof, preferably sodium methylparaben, ethylparaben, salts of ethylparaben, propylparaben, salts of propylparaben, isopropylparaben, salts of isopropylparaben, butylparaben, salts of salts of butylparaben, isobutylparaben, salts of isobutylparaben, DMDM hydantoin, diazolidinyl urea, imidazolidinyl urea, chlorphenesin, disodium EDTA, tetrasodium glutamate diacetate, trisodium ethylenediamine disuccinate, methylglycine N,N-diacetic trisodium salt, glucoheptonic sodium salt, ethanoldiglycinic disodium salt, sodium gluconate, tetrasodium iminodisuccinate, trisodium salt of methylglycine diacetic acid, polyaspartic acid sodium salt, phenoxyisopropanol, chlorhexidine, chlorhexidine digluconate, triclocarban, triclosan, O-phenylphenol, 4-hydroxybenzoic acid, TEA-salicylate, benzyl salicylate, hexamidine, hexamidine diisethionate, dichlorobenzyl alcohol, ethyl benzoate, sodium paraben, isothiazolinones, p-chloro-m-cresol, cetrimonium chloride, cetrimonium bromide, benzethonium chloride, alkyltrimonium bromide, alkyltrimonium chloride, benzalkonium chloride, benzalkonium bromide, myristamidopropyl PG-dimonium chloride phosphate, guar hydroxypropyltrimonium chloride, hydroxypropyl guar hydroxypropyltrimonium chloride, polyquaternium-6, polyquaternium-7, polyquaternium-10, polyquaternium-11, polyquaternium-12, polyquaternium-15, methylheptylglycerin, sodium caproyl/lauroyl lactylate, polyglyceryl-10 laurate, glyceryl caprate, polyglyceryl-2 caprate, raspberry ketone, raspberry ketone methyl ether, caprylyl caprylate, coco-caprylate/caprate, PEG-8 caprylate, dipropylene glycol caprylate, propylene glycol caprylate, pyridoxine dicaprylate, glyceryl caprylate/caprate, methyl glucose caprylate/caprate, polyglyceryl-3 caprate/caprylate/succinate, polyglyceryl-2 caprylate, polyglyceryl-3 caprylate, polyglyceryl-4 caprylate, polyglyceryl-6 caprylate, polyglyceryl-10 caprylate, polyglyceryl-4 caprylate/caprate, polyglyceryl-6 caprylate/caprate, polyglyceryl-10 caprylate/caprate, propanediol dicaprylate/caprate, xylityl caprate/caprylate, xylityl caprylate, xylityl sesquicaprylate, hydroxyethoxyphenyl butanone, or mixtures thereof;
(b) providing additional ingredients of the personal care product; and
(c) mixing the one or more first compounds, the one or more second compounds, and the one or more additional ingredients of the personal care product.

27. The method of claim 26, wherein one or more of the first compounds synergistically improve an antimicrobial effect of one or more of the second compounds, and/or one or more of the second compounds synergistically improve an antimicrobial effect of one or more of the first compounds.

28. The method of claim 26, wherein the personal care product comprises:
(i) from 0.01 to 10 wt. % of the one or more first compounds, based on a total weight of the personal care product; and/or
(ii) from 0.001 to 20 wt. % of the one or more second compounds, based on the total weight of the personal care product.

29. A mixture or composition comprising:
(i) one or more first compounds selected from 3-hydroxypropyl caprylate, 3-hydroxypropyl undecylenate, or a combination thereof;
(ii) one or more second compounds selected from piroctone olamine, climbazole, zinc pyrithione, salicylic acid, salts of salicylic acid, decylene glycol, o-cymen-5-ol, selenium sulfide, coal tar, sulphur, ketoconazole, benzoic acid, salts of benzoic acid, sorbic acid, salts of sorbic acid, lactic acid, salts of lactic acid, dehydroacetic acid, salts of dehydroacetic acid, undecylenic acid, salts of undecylenic acid, levulinic acid, salts of levulinic acid, anisic acid, salts of anisic acid, phytic acid, salts of phytic acid, itaconic acid, salts of itaconic acid, MEA undecylenate, butyloctanoic acid, salts of butyloctanoic acid, citric acid, salts of citric acid, hydroxyacetophenone, ethylhexylglycerin, caprylhydroxamic acid, salts of caprylhydroxamic acid, undecylenoyl glycine, capryloyl glycine, polyglyceryl-3 caprylate, glyceryl undecylenate, glyceryl caprylate, sorbitan caprylate, glyceryl laurate, phenethyl alcohol, tropolones, 1,2-propanediol, 1,2-butanediol, 1,2-pentanediol, 1,2-hexanediol, 1,2-heptanediol, 1,2-octanediol, 1,2-nonanediol, 1,2-decanediol, 1,2-undecanediol, 1,2-dodecanediol, 1,2-tridecanediol, 2,3-pentanediol, 2,3-hexanediol, 2,3-heptanediol, 2,3-octanediol, 2,3-nonanediol, 2,3-decanediol, 2,3-undecanediol, 2,3-dodecanediol, 2,3-tridecanediol, 1,3-propanediol, 1,3-butanediol, 2-methyl-2,4-pentanediol, caprylyl glycol, farnesol, bisabolol, nerolidol, carvacrol, thymol, phenylpropanol, methylbenzyl alcohol, 2-benzylheptanol, 2-methyl 5-cyclohexylpentanol, dimethyl phenylbutanol, PCA ethyl cocoyl arginate, benzyl alcohol, phenoxyethanol, iodopropynyl butylcarbamate, hydroxyethoxyphenyl butanol, ethyl lauroyl arginate HCl, methylchloroisothiazolinone, methylisothiazolinone, methylparaben and salts thereof, preferably sodium methylparaben, ethylparaben, salts of ethylparaben, propylparaben, salts of propylparaben, isopropylparaben, salts of isopropylparaben, butylparaben, salts of salts of butylparaben, isobutylparaben, salts of isobutylparaben, DMDM hydantoin, diazolidinyl urea, imidazolidinyl urea, chlorphenesin, disodium EDTA, tetrasodium glutamate diacetate, trisodium ethylenediamine disuccinate, methylglycine N,N-diacetic trisodium salt, glucoheptonic sodium salt, ethanoldiglycinic disodium salt, sodium gluconate, tetrasodium iminodisuccinate, trisodium salt of methylglycine diacetic acid, polyaspartic acid sodium salt, phenoxyisopropanol, chlorhexidine, chlorhexidine digluconate, triclocarban, triclosan, O-phenylphenol, 4-hydroxybenzoic acid, TEA-salicylate, benzyl salicylate, hexamidine, hexamidine diisethionate, dichlorobenzyl alcohol, ethyl benzoate, sodium paraben, isothiazolinones, p-chloro-m-cresol, cetrimonium chloride, cetrimonium bromide, benzethonium chloride, alkyltrimonium bromide, alkyltrimonium chloride, benzalkonium chloride, benzalkonium bromide, myristamidopropyl PG-dimonium chloride phosphate, guar hydroxypropyltrimonium chloride, hydroxypropyl guar hydroxypropyltrimonium chloride, polyquaternium-6, polyquaternium-7, polyquaternium-10, polyquaternium-11, polyquaternium-12, polyquaternium-15, methylheptylglycerin, sodium caproyl/lauroyl lactylate, polyglyceryl-10 laurate, glyceryl caprate, polyglyceryl-2 caprate, raspberry ketone, raspberry ketone methyl ether, caprylyl caprylate, coco-caprylate/caprate, PEG-8 caprylate, dipropylene glycol caprylate, propylene glycol caprylate, pyridoxine dicaprylate, glyceryl caprylate/caprate, methyl glucose caprylate/caprate, polyglyceryl-3 caprate/caprylate/succinate, polyglyceryl-2 caprylate, polyglyceryl-3 caprylate, polyglyceryl-4 caprylate, polyglyceryl-6 caprylate, polyglyceryl-10 caprylate, polyglyceryl-4 caprylate/caprate, polyglyceryl-6 caprylate/caprate, polyglyceryl-10 caprylate/caprate, propanediol dicaprylate/caprate, xylityl caprate/caprylate, xylityl caprylate, xylityl sesquicaprylate, hydroxyethoxyphenyl butanone, or mixtures thereof.

30. The mixture or composition of claim 29, wherein the one or more first compounds are in an amount sufficient to enhance an antimicrobial effect of one or more second compounds, and/or the one or more second compounds are in an amount sufficient to enhance an antimicrobial effect of one or more first compounds.

31. The mixture or composition of claim 30 comprising:
(i) 1 to 99.9 wt. % of the one or more first compounds, based on a total weight of the mixture or composition; and/or
(ii) 0.1 to 99.9 wt. % of the one or more second compounds, based on the total weight of the mixture of composition.
